SEQUENCE LISTING (110 sequences)

SEQ ID NO: 1 - DNA, 21 bp, Artificial Sequence
  LBc1
cacaatcccactatccttcgc

SEQ ID NO: 2 - DNA, 22 bp, Artificial Sequence
  T-DNA-specific primer FLAG-LB
gacgtaacataagggactgacc

SEQ ID NO: 3 - DNA, 21 bp, Artificial Sequence
  adt1-LP
actgcgatttgtgacataggg

SEQ ID NO: 4 - DNA, 21 bp, Artificial Sequence
  adt1-RP
atatgcacctggtatcccctg

SEQ ID NO: 5 - DNA, 22 bp, Artificial Sequence
  adt2-LP
cagcagctgattcgctgtatgc

SEQ ID NO: 6 - DNA, 22 bp, Artificial Sequence
  adt2-RP
tagcgagagagagtgatgatgc

SEQ ID NO: 7 - DNA, 21 bp, Artificial Sequence
  adt3-LP
cgatcaaacgaaactccaaag

SEQ ID NO: 8 - DNA, 21 bp, Artificial Sequence
  adt3-RP
aattcgaagttgcgtttcaag

SEQ ID NO: 9 - DNA, 22 bp, Artificial Sequence
  adt4-LP
tcaccacgtgaataatgagctc

SEQ ID NO: 10 - DNA, 21 bp, Artificial Sequence
  adt4-RP
tacgtgtagcttaccaaggcg

SEQ ID NO: 11 - DNA, 21 bp, Artificial Sequence
  adt5-LP
tagattagatccgtgcatcgg

SEQ ID NO: 12 - DNA, 21 bp, Artificial Sequence
  adt5-RP
gcttttactggtagggcttcg

SEQ ID NO: 13 - DNA, 21 bp, Artificial Sequence
  adt6-LP
accaaatcactgataagcccc

SEQ ID NO: 14 - DNA, 21 bp, Artificial Sequence
  adt6-RP
atagaaccgccgagagagttc

SEQ ID NO: 15 - DNA, 19 bp, Artificial Sequence
  ADT1 rt-For
gtcagataaccgagcaact

SEQ ID NO: 16 - DNA, 18 bp, Artificial Sequence
  ADT1 rt-Rev
taccaaagccacaaaccc

SEQ ID NO: 17 - DNA, 20 bp, Artificial Sequence
  ADT2 rt-For
tggacactacaatgctctaa

SEQ ID NO: 18 - DNA, 18 bp, Artificial Sequence
  ADT2 rt-Rev
tctcagacctcacctcag

SEQ ID NO: 19 - DNA, 20 bp, Artificial Sequence
  ADT3 rt-For
ccgatggatatgacttcttg

SEQ ID NO: 20 - DNA, 18 bp, Artificial Sequence
  ADT3 rt-Rev
tgacttcacacgttggtt

SEQ ID NO: 21 - DNA, 18 bp, Artificial Sequence
  ADT4 rt-For
tgcggaggttcaagagta

SEQ ID NO: 22 - DNA, 19 bp, Artificial Sequence
  ADT4 rt-Rev
atgcttcttctgtggatgt

SEQ ID NO: 23 - DNA, 19 bp, Artificial Sequence
  ADT5 rt-For
ttggaacatcgaagcactt

SEQ ID NO: 24 - DNA, 20 bp, Artificial Sequence
  ADT5 rt-Rev
aggaatgacgtgtactcttg

SEQ ID NO: 25 - DNA, 19 bp, Artificial Sequence
  ADT6 rt-For
cgaggttcaggagtttaca

SEQ ID NO: 26 - DNA, 20 bp, Artificial Sequence
  ADT6 rt-Rev
tggttacgatgaagttgatg

SEQ ID NO: 27 - DNA, 18 bp, Artificial Sequence
  TIP41 like rt-For
gttcctcctcttgcgatt

SEQ ID NO: 28 - DNA, 18 bp, Artificial Sequence
  TIP41-like rt-Rev
cagttggtgcctcatctt

SEQ ID NO: 29 - DNA, 1746 bp, Arabidopsis thaliana
aagtgtggacctttccgtctgagtaagagtattctgagttttgctcttctttgaaagaccttccagaaatccagactttttgttgtgacttgacgttatctctatgactctccattctttctcaccaaccccatttccccattgagacttttgttgacctaattcctccaaaatctcaccttcacattgctcgatttcagtcctctgaaaaaatctccctgctttgctatggctctgaggtgttttcccatttgggtttgtccccaaacgactcatcatcgttctcctctcatgggtctcgctgaattcgacgctgataaacgccgtcgtttctgtctctgggagtgctcttcttcagcttctcagagagctgtcactgccattgaaggcgagattccttttagccgcgagctcaaaaagtcctctgatgaattgggtttgactcaggagactcaatctctttcttttcacagagacttaagtatgcttcctaaaccgctaactgcaaacagtctttactcttctgatggcgatgattcaaaagtgcgaatttcgtttcaggggataccaggtgcatatagtgagacagcagcactaaaagcatttccaaattgtgaaaccgttccatgtgaacagttcgaagctgcgttccaggctgttgagctctggttggtggataaagcagtattaccgatcgagaattcagtaggtggtagtatccaccgtaattatgatttgcttcttcgtcataggcttcacattgtccaagaagtccacttgcctgtgaatcattgtctcttaggagtgcctggtgttaaaaaggaagatattaaatgtgttctaagccatcctcaggcgcttgatcaatgtgtgaattcacttaacaatttaggcatacagagaatttctgcaaaagatactgccactgctgctcagactgtttcttcaagtggtaagatcgacgtaggagcaatcgcgagtgtacgggctgcaaatatctatggtctagatattcttgctgagaacatacaagatgatgttaacaatgtgactcgctttctgatacttgcgagagagcctatgattccaagaacagatcgaccatataagacaagtatcgtgttctcgctagaagaaggtcctggtgtgctgttcaaggccttggctgtttttgctttacgaagcattaacttatctaaggtaagttccaaggaaaagttggatcctattatcagtaattcattccaaaaatagaaatatatgacgtaacttttgctcttctttagatagaaagtcgtccgcagagaagaagaccgttaagagtagttgatggttctaacaatggaagcgctaagtactttgattacttattctacattgattttgaagcctccatggctgatacacgtgctcagcacgctcttggccatctacaggaattcgcaagtttcatccgtatcctcgggtgttatccaatggatctagtcagataaccgagcaactcttcaattgatttcatgttaccataccgtgaagaacatgtgaatatacccaaacagaagaacagggtttgtggctttggtatacttcaagaatcttctttcaccattttctcattttagaactttagcttcaaactcttcattgttgagacagatacaattgatgtagcattttgttaagaatatgttctgtacattaaacaagttttttcagttaatatcaatgaataaggttgtttgatgc

SEQ ID NO: 30 - protein, 341 aa, Arabidopsis thaliana
MALRCFPIWVCPQTTHHRSPLMGLAEFDADKRRRFCLWECSSSASQRAVTAIEGEIPFSRELKKSSDELGLTQETQSLSFHRDLSMLPKPLTANSLYSSDGDDSKVRISFQGIPGAYSETAALKAFPNCETVPCEQFEAAFQAVELWLVDKAVLPIENSVGGSIHRNYDLLLRHRLHIVQEVHLPVNHCLLGVPGVKKEDIKCVLSHPQALDQCVNSLNNLGIQRISAKDTATAAQTVSSSGKIDVGAIASVRAANIYGLDILAENIQDDVNNVTRFLILAREPMIPRTDRPYKTSIVFSLEEGPGVLFKALAVFALRSINLSKVSSKEKLDPIISNSFQK

SEQ ID NO: 31 - DNA, 1660 bp, Arabidopsis thaliana
aagtgtggacctttccgtctgagtaagagtattctgagttttgctcttctttgaaagaccttccagaaatccagactttttgttgtgacttgacgttatctctatgactctccattctttctcaccaaccccatttccccattgagacttttgttgacctaattcctccaaaatctcaccttcacattgctcgatttcagtcctctgaaaaaatctccctgctttgctatggctctgaggtgttttcccatttgggtttgtccccaaacgactcatcatcgttctcctctcatgggtctcgctgaattcgacgctgataaacgccgtcgtttctgtctctgggagtgctcttcttcagcttctcagagagctgtcactgccattgaaggcgagattccttttagccgcgagctcaaaaagtcctctgatgaattgggtttgactcaggagactcaatctctttcttttcacagagacttaagtatgcttcctaaaccgctaactgcaaacagtctttactcttctgatggcgatgattcaaaagtgcgaatttcgtttcaggggataccaggtgcatatagtgagacagcagcactaaaagcatttccaaattgtgaaaccgttccatgtgaacagttcgaagctgcgttccaggctgttgagctctggttggtggataaagcagtattaccgatcgagaattcagtaggtggtagtatccaccgtaattatgatttgcttcttcgtcataggcttcacattgtccaagaagtccacttgcctgtgaatcattgtctcttaggagtgcctggtgttaaaaaggaagatattaaatgtgttctaagccatcctcaggcgcttgatcaatgtgtgaattcacttaacaatttaggcatacagagaatttctgcaaaagatactgccactgctgctcagactgtttcttcaagtggtaagatcgacgtaggagcaatcgcgagtgtacgggctgcaaatatctatggtctagatattcttgctgagaacatacaagatgatgttaacaatgtgactcgctttctgatacttgcgagagagcctatgattccaagaacagatcgaccatataagacaagtatcgtgttctcgctagaagaaggtcctggtgtgctgttcaaggccttggctgtttttgctttacgaagcattaacttatctaagatagaaagtcgtccgcagagaagaagaccgttaagagtagttgatggttctaacaatggaagcgctaagtactttgattacttattctacattgattttgaagcctccatggctgatacacgtgctcagcacgctcttggccatctacaggaattcgcaagtttcatccgtatcctcgggtgttatccaatggatctagtcagataaccgagcaactcttcaattgatttcatgttaccataccgtgaagaacatgtgaatatacccaaacagaagaacagggtttgtggctttggtatacttcaagaatcttctttcaccattttctcattttagaactttagcttcaaactcttcattgttgagacagatacaattgatgtagcattttgttaagaatatgttctgtacattaaacaagttttttcagttaatatcaatgaataaggttgtttgatgc

SEQ ID NO: 32 - protein, 392 aa, Arabidopsis thaliana
MALRCFPIWVCPQTTHHRSPLMGLAEFDADKRRRFCLWECSSSASQRAVTAIEGEIPFSRELKKSSDELGLTQETQSLSFHRDLSMLPKPLTANSLYSSDGDDSKVRISFQGIPGAYSETAALKAFPNCETVPCEQFEAAFQAVELWLVDKAVLPIENSVGGSIHRNYDLLLRHRLHIVQEVHLPVNHCLLGVPGVKKEDIKCVLSHPQALDQCVNSLNNLGIQRISAKDTATAAQTVSSSGKIDVGAIASVRAANIYGLDILAENIQDDVNNVTRFLILAREPMIPRTDRPYKTSIVFSLEEGPGVLFKALAVFALRSINLSKIESRPQRRRPLRVVDGSNNGSAKYFDYLFYIDFEASMADTRAQHALGHLQEFASFIRILGCYPMDLVR

SEQ ID NO: 33 - DNA, 1518 bp, Arabidopsis thaliana
aaaacaacatcaatggcaatgcacactgttcgattgtcgccggcgactcaacttcacggcggtatctctagcaatctgtcaccacccaatcgaaaacctaataattccatcgtccgatacggatgcggatcttcgaagcgtttccgtatcgtaacggtcttggcgtcactccgggaaaacgatgcaaacggaagggataactccgtgagagctatggaagtgaagaagatctttgaagactcgcctctgcttcctaagccgttatcatcgaaccagctcacggaatctgtctctaatggctcccgcgttcgtgttgcgtatcagggagtacgaggtgcatacagcgaatcagctgctgagaaggcttatccaaactgtgaagctgttccatgcgaagagtttgacactgcttttgaggcggttgagcggtggcttgttgacagagctgttttaccgattgagaactctttaggtggaagcatccatagaaattatgatcttctgctgcgacacaatttgcatattgtaggcgaagtcaagctagctgttcggcactgtttgttggctaaccacggtgtaaatattgaagatttgagaagggtgcttagccatccacaggctcttgcccaatgtgaaaacacgttaaccaaattggggttggtcagagaagcagtagatgatactgctggcgctgcgaagcaaattgcattcgaaaatttaaatgatgcagctgcagttgccagtgaaaaagctgcaaagatatatggtttgaacatagtcgctaaagatatccaggatgactgtgataatgttacaaggtttctaatgctcgcaagagaacccattatccctggaactaacagactctttaagacaagtatagttttctcgttagaggaagggcctggagtacttttcaaagcgcttgctgtgtttgcccttcggcaaatcaacctcacaaagattgaaagccgccctttaaggaaacatcctctccgagcatctggaggactaaaatactttgactatcttttctatgtggactttgaagcatctatggctgatgaagttgctcaaaacgcacttaggcatctcgaggagtttgctacctttttgcgggtactgggaagctacccagtggacactacaatgctctaaacatttcaaacatcttcttgtttgtgacagagatccgcttgatgatatcataatcctggtataaggagctatatccctgaggtgaggtctgagaggagtcaccaccttacaagacattccgctcttgttacaaattctgtttagttttccaaaaaagatcatctttgtttttagtaagaatgtagtcactaagtcagatatcaatgacaaataccaaagaactagtgttttttcgtgtttcatccatacatagatacttatcctgtgtcttttttagaaatttctttttgtatgacgtttcaaaattttgattgactattgttagtggtatatctttgagttaataaacgagatataatc

SEQ ID NO: 34 - protein, 381 aa, Arabidopsis thaliana
MAMHTVRLSPATQLHGGISSNLSPPNRKPNNSIVRYGCGSSKRFRIVTVLASLRENDANGRDNSVRAMEVKKIFEDSPLLPKPLSSNQLTESVSNGSRVRVAYQGVRGAYSESAAEKAYPNCEAVPCEEFDTAFEAVERWLVDRAVLPIENSLGGSIHRNYDLLLRHNLHIVGEVKLAVRHCLLANHGVNIEDLRRVLSHPQALAQCENTLTKLGLVREAVDDTAGAAKQIAFENLNDAAAVASEKAAKIYGLNIVAKDIQDDCDNVTRFLMLAREPIIPGTNRLFKTSIVFSLEEGPGVLFKALAVFALRQINLTKIESRPLRKHPLRASGGLKYFDYLFYVDFEASMADEVAQNALRHLEEFATFLRVLGSYPVDTTML

SEQ ID NO: 35 - DNA, 1508 bp, Arabidopsis thaliana
aaaacaacatcaatggcaatgcacactgttcgattgtcgccggcgactcaacttcacggcggtatctctagcaatctgtcaccacccaatcgaaaacctaataattccatcgtccgatacggatgcggatcttcgaagcgtttccgtatcgtaacggtcttggcgtcactccgggaaaacgatgcaaacggaagggataactccgtgagagctatggaagtgaagaagatctttgaagactcgcctctgcttcctaagccgttatcatcgaaccagctcacggaatctgtctctaatggctcccgcgttcgtgttgcgtatcagggagtacgaggtgcatacagcgaatcagctgctgagaaggcttatccaaactgtgaagctgttccatgcgaagagtttgacactgcttttgaggcggttgagcggtggcttgttgacagagctgttttaccgattgagaactctttaggtggaagcatccatagaaattatgatcttctgctgcgacacaatttgcatattgtaggcgaagtcaagctagctgttcggcactgtttgttggctaaccacggtgtaaatattgaagatttgagaagggtgcttagccatccacaggctcttgcccaatgtgaaaacacgttaaccaaattggggttggtcagagaagcagtagatgatactgctggcgctgcgaagcaaattgcattcgaaaatttaaatgatgcagctgcagttgccagtgaaaaagctgcaaagatatatggtttgaacatagtcgctaaagatatccaggatgactgtgataatgttacaaggtttctaatgctcgcaagagaacccattatccctggaactaacagactctttaagacaagtatagttttctcgttagaggaagggcctggagtacttttcaaagcgcttgctgtgtttgcccttcggcaaatcaacctcacaaagattgaaagccgccctttaaggaaacatcctctccgagcatctggaggactaaaatactttgactatcttttctatgtggactttgaagcatctatggctgatgaagttgctcaaaacgcacttaggcatctcgaggagtttgctacctttttgcgggtactgggaagctacccagtggacactacaatgctctaaacatttcaaacatcttcttgagatccgcttgatgatatcataatcctggtataaggagctatatccctgaggtgaggtctgagaggagtcaccaccttacaagacattccgctcttgttacaaattctgtttagttttccaaaaaagatcatctttgtttttagtaagaatgtagtcactaagtcagatatcaatgacaaataccaaagaactagtgttttttcgtgtttcatccatacatagatacttatcctgtgtcttttttagaaatttctttttgtatgacgtttcaaaattttgattgactattgttagtggtatatctttgagttaataaacgagatataatc

SEQ ID NO: 36 - protein, 381 aa, Arabidopsis thaliana; identical to SEQ ID NO: 34

SEQ ID NO: 37 - DNA, 1521 bp, Arabidopsis thaliana
acaaaacatttccataaccaaaaaacctcaaaataaaaaaatgagaactctcttaccttcccatactccggcaacggtaacaacggcggcgagaagaagacacgtcatacattgcgccggtaagagatctgactctttctccattaattccagtagctccgattggcaaagctcctgcgccattctctcaagcaaagttaactcacaagaacaatctgaatccttatcttccaattccaatggatcatcatcctaccacgtttccgccgtcaacggtcacaacaacggcgccggagtctcagatttgaatctagttccattcaacaacaaccaatcgattcaatcgaagaagcctttaagtatttcagatctatctccagctccgatgcatggatctaacctccgtgtagcttatcaaggtgttcccggagcttactccgaagccgccgctggtaaagcttatcctaactgtcaagcgattccttgcgatcaattcgaagttgcgtttcaagcggttgagctttggatcgccgatcgcgctgttttacccgtcgaaaactcactcggcggttcgattcatcgtaattacgatcttctcctccgtcaccgtcttcacatcgtcggcgaagttcagcttccggttcatcattgtctcattgctcttcccggtgtacggaaagagtttctcactcgtgtgatctctcatcctcaaggactagctcaatgcgaacacacgttgactaaactcggtctcaacgtagctcgtgaagctgttgatgataccgccggtgcggcggagtttatcgctgcgaataatatccgtgacaccgccgcgattgcgagtgcacgcgcggcggagatctacggattggagattttggaagatgggatacaagatgacgcgagtaacgtcacgcgctttgtgatgttggcgcgtgaaccgattataccgagaactgatcggccgtttaagacgagtatagtgtttgctcacgagaaaggcacgtgcgttttgtttaaggttctctctgcttttgcgtttaggaacattagtttgactaagattgagtctaggccgaaccataatgttcctattaggctagtcgatgaggctaatgtgggcacggctaagcattttgagtatatgttttatatcgacttcgaggcttccatggccgagtcacgtgctcagaatgcgctttctgaggttcaagagtttacgtcgtttttacgggttttggggagttatccgatggatatgacttcttggtcaccatcgtcttcgtcctcttcgtcatcaacattttcattgtgacaacactaagatcataaccaacgtgtgaagtcaattgtgtatgtaaattggagaagaaagttgtaaacataattttggtgaaaataaattacttatttctccaaagatcaatcttttgtattttgctgtttacattattatactttggagtttcgtttgatcgtagttttattatgaaattgcaagttttgagttgatttattctc

SEQ ID NO: 38 - protein, 424 aa, Arabidopsis thaliana
MRTLLPSHTPATVTTAARRRHVIHCAGKRSDSFSINSSSSDWQSSCAILSSKVNSQEQSESLSSNSNGSSSYHVSAVNGHNNGAGVSDLNLVPFNNNQSIQSKKPLSISDLSPAPMHGSNLRVAYQGVPGAYSEAAAGKAYPNCQAIPCDQFEVAFQAVELWIADRAVLPVENSLGGSIHRNYDLLLRHRLHIVGEVQLPVHHCLIALPGVRKEFLTRVISHPQGLAQCEHTLTKLGLNVAREAVDDTAGAAEFIAANNIRDTAAIASARAAEIYGLEILEDGIQDDASNVTRFVMLAREPIIPRTDRPFKTSIVFAHEKGTCVLFKVLSAFAFRNISLTKIESRPNHNVPIRLVDEANVGTAKHFEYMFYIDFEASMAESRAQNALSEVQEFTSFLRVLGSYPMDMTSWSPSSSSSSSSTFSL

SEQ ID NO: 39 - DNA, 1693 bp, Arabidopsis thaliana
agcttgagcttctctctcaacgaaagaactcaagctttccaacactataaatctataatacagcttttattctcaaattttgtttgtcttaccatcattaccaaaccatgcaagccgcaacgtcgtgtgatctcaagttccgatcaacagatccgacgtctaggaacaaatgtttctcccacgcgattccaaagcgcgtggctgttacgtgcggttataggtcggagtcgtttagcttccctaacggtgtctccgtgagtcgatctgattggcaaagctcatgtgccattttatctagtaaagttgcttctgttgaaaataccggtggtttagcggacaaaatcgccgccgttaatggtcacacgaacggctctgtgaatcttggtctcgtcgccgttgagtcaactaacggaaagttagctcctgctcagccgttgactattactgatctatctccggcaccgttgcatggttctagtctacgtgtagcttaccaaggcgttcccggagcttactcggaagcagctgccggaaaagcttatcccaattgcgacgccattccttgtgaccagtttgacgttgcttttcaggcggtggagctttggatcgctgatcgagctgtgcttccggtggagaactcactcggtggttcgatccatcgaaactatgatcttcttctccgtcaccgtcttcacatcgttggagaagttcagattccggtccaccattgcctcctcgcactcccgggagtccgaactgactgtgtttcgcgggtgatctctcacccgcaagccctagcgcagacggaacactccctcgacgtccttacaccacatgcagcgcgtgaggctttccatgacacagcggctgctgcggagtatatctcagctaacgacctacatgacacggcggctgtggcgagcgcacgcgccgctgagctttacaacctccagatattagcggatgggattcaagacgacccggggaacgtcactcgtttcctaatgttggcgcgtgagcctataatcccacgcacggaccggccattcaagactagcatcgtctttgctgcccaagagcacaaaggaactagcgtcctctttaaagtcctctcggctttcgctttcagagacattagcctgaccaagatcgagtcccggcctcaccataaccgcccgcttagggttgttggtgacgggagctttgggacgtcgaagaacttcgagtacatgttttacgtggattttgaggcatcgatggctgagccgcgtgcacagaacgcgcttgcggaggttcaagagtacacgtcgttcctaagggtgctgggaagttaccccatggatatgacaccatggtccatgacatccacagaagaagcatgaccccatcgccaaaaacaaactcaccaataaatttattaaattattctgttctttctttttttgtgaaaattgtagtatacattgaaattaaaccgtaaatgtagagctcattattcacgtggtgattaggagaaaacaaaattttatttgtctgttactagctaccagatatatcttacattcttacttaatagttatgaacattgtggtttattcgcagaatctttttgagttccttggttttctttggtaaattcactcgattttaaccaagaaaagcaaaaatgattggcattaccaaaaacaaagac

SEQ ID NO: 40 - protein, 424 aa, Arabidopsis thaliana
MQAATSCDLKFRSTDPTSRNKCFSHAIPKRVAVTCGYRSESFSFPNGVSVSRSDWQSSCAILSSKVASVENTGGLADKIAAVNGHTNGSVNLGLVAVESTNGKLAPAQPLTITDLSPAPLHGSSLRVAYQGVPGAYSEAAAGKAYPNCDAIPCDQFDVAFQAVELWIADRAVLPVENSLGGSIHRNYDLLLRHRLHIVGEVQIPVHHCLLALPGVRTDCVSRVISHPQALAQTEHSLDVLTPHAAREAFHDTAAAAEYISANDLHDTAAVASARAAELYNLQILADGIQDDPGNVTRFLMLAREPIIPRTDRPFKTSIVFAAQEHKGTSVLFKVLSAFAFRDISLTKIESRPHHNRPLRVVGDGSFGTSKNFEYMFYVDFEASMAEPRAQNALAEVQEYTSFLRVLGSYPMDMTPWSMTSTEEA

SEQ ID NO: 41 - DNA, 1819 bp, Arabidopsis thaliana
caaataaaaacaactttataatataaacaacaaatatcgttcaggaaaatcaaatgaaatgtggtaggtgaaggatttgacggaactacccttcaagacacgtcaagactttctttcttttacaacattccactatataaacacttccttctccaccatcatctctcagcttgcaagttctctctgaaccatagaactccaagcttgaactgtactctactcgtcgtttatatcttactcttttgtttcctcttaccagaaaaaataaataaacagcaattttttcatttttcgactcaaatcatgcaaaccatttcgcctgcgttctcgtgcgatctcaaatccgtaattcaaccgaatctaacggctaagaaagcgcgttactctcacgtaaatggaaagcgcgtgtctgttcgttgcagttacaggtctgagtcgtttagcttccctaacggtgttggctcaagccgagccgattggcaaagctcatgcgccatcttagccagtaaagtagtttcggctgagaattcaagctccgtcgccgtcgttaacggacacagtaacggctccgttgatttaagccttgttccgtcaaagtcgcagcacaacggaaagcctggattgattcagccgttaacgataacagatctatctcccgcgccgtctcatggatctactcttcgtgtcgcgtatcaaggagttccgggagcgtactccgaagcggccgccggaaaagcttacccaaactccgaagctattccttgtgatcaattcgacgttgcgtttcaggcggtggagctttggatcgccgatcgtgctgttctcccagttgagaactctctcggtggttcgattcatagaaattacgatcttctcctccgtcaccgtctccacatcgtcggagaagttcagatcccggttcaccactgtctcttagctcttcccggagttcgtacggattgcatcacgcgagtgatttcgcatcctcaagctctggctcagacggaaggatcgctcaacaaactcactccaaaagcagcgattgaagcgtttcacgacacagccgcggcggcggaatacatcgctgctaacaacctccacgacacggcggcggtagcgagcgcaagagcagctgagctctacggacttcagattctcgccgacgggatccaagacgacgcaggaaacgtcacgcgcttccttatgttggcgcgtgatccgatcattccccgtaccgatcgtccgtttaaaacgagtatcgttttcgcagctcaagaacataaaggaactagcgttttattcaaagtgctttccgcgtttgcgtttcgaaacattagtttgacgaaaatcgaatcgcggccgcatcagaattgtcccgttagagtcgtaggcgacgaaaacgttggaacatcgaagcactttgagtacacattctacgtcgacttcgaagcgtcgatggcggaggcgcgtgcacaaaacgcgttagcggaggtgcaagagtacacgtcattcctcagggtgctgggaagttacccaatggatatgacgccatggtccacgttacctagcgaagacgtatgacacaattaaaaaatttatctgaattttaaataacattatggtaaaattatcataagtaaaatacatggaaactaaagtagaatttgtagaactcgttttgcatgtgaagtgggaggataatattattttatttctcttaaaccaaatattttgtgtgtatgtatttgctactattggaacaagaccggtttgtagtttggttctgatgattacaattgaagatatttcttaagctaag

SEQ ID NO: 42 - protein, 425 aa, Arabidopsis thaliana
MQTISPAFSCDLKSVIQPNLTAKKARYSHVNGKRVSVRCSYRSESFSFPNGVGSSRADWQSSCAILASKVVSAENSSSVAVVNGHSNGSVDLSLVPSKSQHNGKPGLIQPLTITDLSPAPSHGSTLRVAYQGVPGAYSEAAAGKAYPNSEAIPCDQFDVAFQAVELWIADRAVLPVENSLGGSIHRNYDLLLRHRLHIVGEVQIPVHHCLLALPGVRTDCITRVISHPQALAQTEGSLNKLTPKAAIEAFHDTAAAAEYIAANNLHDTAAVASARAAELYGLQILADGIQDDAGNVTRFLMLARDPIIPRTDRPFKTSIVFAAQEHKGTSVLFKVLSAFAFRNISLTKIESRPHQNCPVRVVGDENVGTSKHFEYTFYVDFEASMAEARAQNALAEVQEYTSFLRVLGSYPMDMTPWSTLPSEDV

SEQ ID NO: 43 - DNA, 1445 bp, Arabidopsis thaliana
ctacattctcgactttgctatttcattgagcttgaataaccaattttaagacgccattgctaaaacatgaaagctctatcatcttcttctccaatattaggtgctagccagccggcgacggcgactgcgcttatcgcgcgatctggtcgttctgaatggcagagctcctgcgctattctaacaagcaaggttatttctcaagaagaatctgaatccttacctgtgcctccagtttccggcggagttgatcatctcaacggccataacagcgccgccgcacgagttccaggaatgaatcttgttccgattgagaaatctgatagcaatccgttggttccacagcatcgtcataatccactgaaaccccttagcatgacggatctctcgccggctccgatgcacggatctaatctacgcgtcgcttatcaaggtgttcccggtgcttactctgaagcagccgctggtaaagcttatcctaattgtcaagctataccttgcgatcaatttgaggttgcgtttcaagcggtggagctttggatcgcagatcgtgctgttttaccggtggagaactctctcggcggttctatccaccgtaattacgatctcctcctccgtcaccgtcttcatatagtcggcgaagttcaactccctgttcatcactgtttactggctcttcccggcgttcgtaaagagtttctcacacgtgtgatctcacaccctcaaggtctcgctcagtgtgagcacacgctcacaaagctcggacttaatgtcgcacgtgaggcagttgatgacaccgcaggagccgctgagttcatcgcctctaacaacctccgcgacacagccgctatcgcgagtgcacgcgccgccgagatatacgggttggagattttggaagatgggattcaggacgacgtgagtaacgtgacgcgcttcgtgatgctggcgcgtgagccaatcatacctagaacagatagaccgttcaagactagcattgtctttgcgcacgagaaaggaaccagcgtgcttttcaaggtgctctctgctttcgccttcagagacattagcttgacgaagatcgaatcgaggccgaatcacaaccgtccgatcagggtcgtggatgatgccaatgtcgggacggcgaagcattttgagtacatgttttatgttgacttcgaggcctcgatggcggaggcgcgtgctcagaacgcgcttgccgaggttcaggagtttacatcgttcttgcgggtgcttgggagttatcccatggatatgactccttggtcaccaacatcatcaacttcatcgtaaccatactatagtctatacacacatgaaaatgtaaattttggagaagataacaaagttttatatttactgatccattctgtttggtgttttacatttgattgaattcgtataaatcaataaacaattggtaaaaattt

SEQ ID NO: 44 - protein, 413 aa, Arabidopsis thaliana
MKALSSSSPILGASQPATATALIARSGRSEWQSSCAILTSKVISQEESESLPVPPVSGGVDHLNGHNSAAARVPGMNLVPIEKSDSNPLVPQHRHNPLKPLSMTDLSPAPMHGSNLRVAYQGVPGAYSEAAAGKAYPNCQAIPCDQFEVAFQAVELWIADRAVLPVENSLGGSIHRNYDLLLRHRLHIVGEVQLPVHHCLLALPGVRKEFLTRVISHPQGLAQCEHTLTKLGLNVAREAVDDTAGAAEFIASNNLRDTAAIASARAAEIYGLEILEDGIQDDVSNVTRFVMLAREPIIPRTDRPFKTSIVFAHEKGTSVLFKVLSAFAFRDISLTKIESRPNHNRPIRVVDDANVGTAKHFEYMFYVDFEASMAEARAQNALAEVQEFTSFLRVLGSYPMDMTPWSPTSSTSS

SEQ ID NO: 45 - DNA, 1863 bp, Oryza sativa
aaagaaaagaaaaaaaaatctttgcccggcgcggtcgcggcgccacacgcgtgacacacacccgcaccgcacgccgagagccgacaccgccgcgccgccgctccccttccccaccgcgccgcgcgcggcggcgatggattccacggcctgcctccgcctcccgttcctccccgcccggacccgcccctcctcctcctcctcgtcgtctccccgacgcgccgctcgcgcgtcctccatcaaatgctgcaccgccgcctccgacgccggtgccagttcggcctccatctcttccgcgtcgccgcggcggccggacgtggtgaacggcgtggggcccgccggcgtagacggcctcgcgggtccgcccgtgcccgtaccggactcccccgcccccgcctcccgtgatctccactggctgcctcgccctctcaccagcgccgacctgatgggcgtcagcggggaggggctcaaggttgcctaccagggttgccctggagcgtacagcgaggcggcagcgaagaaggcgtacccaaattgccagactgtgccctgtgagcacttcgatactgcattcaaggctgttgaaaactggttagctgacagggcagtattgccacttgagaattcattgggtggtagcatacataggaactttgaccttttacttcgtcatagattgcacattgttggagaggtccgtcttgctgttcgtcattgcctgttagccaatcccggtgtcaagattgaaaacttgaaaagtgcaatgagccatccccaggctcttgcacaatgtgagcacacattgacagagttcggaattgaacatagagaagctgttgatgatactgctggtgcagcaaagactgtagcagaacaaaatctccaagataccggtgcgattgccagttcactggcagctgaactttatggactgaatgttcttgctgaaaatattcaggatgacaaagataatgttacccgttttatgatgctggctcgagaaccaattatacctcgcaccgataaaccattcaagactagcatagtcttttcacttgaagaaggacctgggcaactctttaaggcacttggggtgtttgcactgagggaaatcaacctaaccaagatagaaagccgcccacacaagaaaaggcctcttcgtataactgacgacagcttttccacaccgtcaaagcagtttgactacctcttctatatggaccttgaggcatcaatggctgatccaaaaactcagaatgctcttggaaatttgaaggagtttgcaacctttttaagagtccttggaagctatcctacagatgtaaatgaagcttaaatagcaagcaccttctgcatatcacatgctagattaccagggatggattgagcgtgggctgtcaatttcacagcgagttgggaagtcatcaataatttctagactgatgctaatttgaagagcaataaagcatatccatgtattatcggcaaggaagctgcatgtgacttggttagaagatggaaaagaacaaaatagtgggttttacgttttactaagagctttatagtgatatacttaccaccatccatgccctatagtccttatgttgtaacttatgttcttctaagcaatcatgttcaacagaggtagagctcaaagggttgaatgtttggtaggtgccggtggttataggtagtggtgtagaacaatttcgctcgttcccaggggatgcgctggtcaaacccttcagtcggcttgccacccaactgttgtattttcttgtcctgtatgtgctcatggactcttagtcgtgagctcgtagcagatagttcaatcatgtttccctcgtgccaacatggcacagtacc

SEQ ID NO: 46 - protein, 399 aa, Oryza sativa
MDSTACLRLPFLPARTRPSSSSSSSPRRAARASSIKCCTAASDAGASSASISSASPRRPDVVNGVGPAGVDGLAGPPVPVPDSPAPASRDLHWLPRPLTSADLMGVSGEGLKVAYQGCPGAYSEAAAKKAYPNCQTVPCEHFDTAFKAVENWLADRAVLPLENSLGGSIHRNFDLLLRHRLHIVGEVRLAVRHCLLANPGVKIENLKSAMSHPQALAQCEHTLTEFGIEHREAVDDTAGAAKTVAEQNLQDTGAIASSLAAELYGLNVLAENIQDDKDNVTRFMMLAREPIIPRTDKPFKTSIVFSLEEGPGQLFKALGVFALREINLTKIESRPHKKRPLRITDDSFSTPSKQFDYLFYMDLEASMADPKTQNALGNLKEFATFLRVLGSYPTDVNEA

SEQ ID NO: 47 - DNA, 1502 bp, Oryza sativa
gcagatggaaacgcaacgctgttgctagcccagagaaagaaagacgaggaaaggaggagcaggcggcggcgtccagggtccagcagcatagcattgagcttgagcatggtttccccttcgcttcggatccccgcggccgcgcgcgtcttcgatcccgccatcaacacccgctgccctccgccccacgccgtcgtcatgcgcatgcgcagtcgcagccgccgttccatcgctgcctccgcttcgccgccgggggacccctccatccgcgaccccatctcactgccaaggccgctcaccagcgcggatctgatggaggccagcggagacggtttgaaggtcgcataccagggatgcccgggtgcctacagtgaggccgccgccaagaaggcgtacccgagttgccacaccgtgccctgcgaatacttcgagaccgcctttcaggctgtcgaaaattgggtggctgaccgcgcggtcctgccacttgagaattccttgggtggtagcatccatcgaaactatgaccttctacttcgccataggctgcacattgttggtgaggtgcgccttgcagttcgccattgcttgttagcaaatcgtggtgtgaagatccaaaatttgagaagtgccatgagccatcctcaagctcttgcacagtgtgaacaaacactgacaaagttaggcatcgagcatagagaagctgttgacgatacagcaggtgcagcaaagcttattgcagaacaaaagctccaggacactggagcagttgctagttcattggcagctcaactttatggactggatattcttgcagaaaatattcaggatgacaccgataatgtaacacgctttatgatgctggctcgggaacccattattcctcgtactgataagccattcaagactagcatagtcttttctttggaagaagggcctgggcaactatttaaggcgctggcagtgtttgcactgagaaaaattaacctcaccaagatggaaagccgtccacacaagaaaaagcctctacgtatagctgatgataattgttctgcaccattgaagcatttcgactacctcttctatgtagatcttgaggcgtcaatggctgatccaaatgctcaaaatgctctggcaaacttaaaggagttcgcaacctttctaagagttcttgggagctatcctaccgatgtcagtgaagcatgaattgtcaagtacctgttttgaatatacttggaccagcgttatcatgaatgagcttacgattggttctatcgccaacttggaagcaccgatagcagaagtggtactaacttggagggtaacgatatgacatattgatagagagaatggcacaaaggtgcacgatgttttgttcttgtttttaagttctcattgttgtgttagtgacgatttccttttgaaaggggcctttatgcaatatcccacttacccaaaagaacagtgatgagatatcaaagctaaggccaaagaattggttgtttgtc

SEQ ID NO: 48 - protein, 364 aa, Oryza sativa
MVSPSLRIPAAARVFDPAINTRCPPPHAVVMRMRSRSRRSIAASASPPGDPSIRDPISLPRPLTSADLMEASGDGLKVAYQGCPGAYSEAAAKKAYPSCHTVPCEYFETAFQAVENWVADRAVLPLENSLGGSIHRNYDLLLRHRLHIVGEVRLAVRHCLLANRGVKIQNLRSAMSHPQALAQCEQTLTKLGIEHREAVDDTAGAAKLIAEQKLQDTGAVASSLAAQLYGLDILAENIQDDTDNVTRFMMLAREPIIPRTDKPFKTSIVFSLEEGPGQLFKALAVFALRKINLTKMESRPHKKKPLRIADDNCSAPLKHFDYLFYVDLEASMADPNAQNALANLKEFATFLRVLGSYPTDVSEA

SEQ ID NO: 49 - DNA, 1669 bp, Oryza sativa
attctcttctcacgccggcgccgcggcgtctcgtctcctcccaccgcaacctttcttcttttgcttagctttcacttacactacccctcgccgcgcctcgccgcgcgacgcggccctttcccgccaccaacccctcgccgcgcccgcctcgcctccggagaccgccggaggatggaaacctcgcccgcgctgcgccccgcccacatccccagatccacggccgtccgcccgccgtgcgccacccctttctcccgcgcccactggcacgccacctgcgccgccatccgccgagtcccgcgggtgaacggcgacagcaacagtagcatcaagcccgccctcgccgaccacgccgcgccgccgctcgacctggatctgctccccgtctccaacctccctcgcccgctcaccatcaccgacctctcccccgcgcccatgcacggcagccagctgcgcgtcgcctaccagggtgtcccgggggcatacagcgaggcggcggcggcgaaggcctacccctcctgcgacgccatcccctgcgaccagttcgaggtggccttccaggccgtcgagctctggatcgccgaccgcgccgtcctccccgtcgagaactccctcggcggcagcatccaccgcaactacgacctcctcctccgccaccgcctccacatcgtcggcgaggtccagctccccgtccaccactgcctcctcgcgctcccgggcgtccgcagggacctcctcacccgcgtcatctcccacccgcaggcgctcgcccagtgcgagctcaccctcaacgccatgggcctcaacgtcgcccgcgaggccttcgacgacaccgcggccgccgccgagcacgtcgccgccgcggggctccgcgacaccgccgccatcgcgtcctcccgcgccgccgagctgtacgggctccaggtgctcgcggacggcatccaggacgacgccgggaacgtgacgcggttcgtgatgctggcgcgggagcccatcatcccgcgaacggaccggccattcaagacgagcatcgtgttcgcgcacgacagggagggcacgtcggtgctcttcaaggtgctgtcggccttcgcgttccgggacatcagcctcaccaagatcgagagccgcccacaccgccaccgccccatccggctcgtcgacgacgccaacgtgggcacggccaagcacttcgagtacatgttctacatcgacttccaggcgtccatggcggaggtcagggcgcagaacgcgctgtcggagatacaggagttcacctccttcctccgggtgctcggcagctaccccatggacatgacgccatgggagtattcctcctcatcgtcaccatcaccggcgcccggcggcgacaccagcagataatgctgatcgatcgacaagactgatgctcaaattctgttagtgactaattgcttctttctcttctttttttttctgtcaaattagagaaaggggaggaagactctcttcctcatgtttactttcggttgcggatgatactctgattaattagtcaaagttatgctcggtgaggatcggagtagatgagatgccatagcggagaggggagacagcgatcccatctcattaccagaaatgaatttttgtactctgaacattagaaaaaagggaatgaattaggataactgatggt

SEQ ID NO: 50 - protein, 401 aa, Oryza sativa
METSPALRPAHIPRSTAVRPPCATPFSRAHWHATCAAIRRVPRVNGDSNSSIKPALADHAAPPLDLDLLPVSNLPRPLTITDLSPAPMHGSQLRVAYQGVPGAYSEAAAAKAYPSCDAIPCDQFEVAFQAVELWIADRAVLPVENSLGGSIHRNYDLLLRHRLHIVGEVQLPVHHCLLALPGVRRDLLTRVISHPQALAQCELTLNAMGLNVAREAFDDTAAAAEHVAAAGLRDTAAIASSRAAELYGLQVLADGIQDDAGNVTRFVMLAREPIIPRTDRPFKTSIVFAHDREGTSVLFKVLSAFAFRDISLTKIESRPHRHRPIRLVDDANVGTAKHFEYMFYIDFQASMAEVRAQNALSEIQEFTSFLRVLGSYPMDMTPWEYSSSSSPSPAPGGDTSR

SEQ ID NO: 51 - DNA, 1662 bp, Oryza sativa
agcttctcgcgctgaagcttcaaacctacccaaattaccaaactatactactgctcgacacggttcgttttttttcaccgacccaagaagcagagaaagaaaggaggtgagaaatctggtgggtgtggtgatggccgcggcgagtttgatcaaggcgcccgtggggcagaatcctgcgaggatgggcgcggggaggagcagcggaggaggaggggtggtcaggtgctcgctgcagggcgccgtggtgggcggccgggcggagtggcagagcagctgcgccgtgctgtccagcaaggtggccgcgctcggggcggcgtccccgcacgccgcggcgccaagcttcgttaatgggcatgtcgcgccgctggtcccggagcagcaggcggcggcggaggacggcggggcggtgctggatttggtgcccgtgagcagcgttaatggcgggggcgtcgccaagaacctgccgcagccgctgcggatctcggatctgtcgccggcgccgatgcacgggtcgcagctgcgcgtggcgtaccagggcgtccccggtgcgtacagcgagaaggcggccgggaaggcgtacccgggctgcgatgccatcccctgcgaccagttcgaggtggcgttctcggccgtggagctgtggatcgccgaccgcgccgtgctccccgtggagaactcgctgggcggcagcatccaccgcaactacgacctcctgctcaggcaccggctccacatcgtcggcgaggtgcagctccccgtgcaccactgcctcatggcgctcccgggggtccgcaaggagtgcctcacccgcgtgatgagccacccgcaggcgctggcgcagtgcgagcacacgctcaccgccatggggctcaacgtcgtccgcgaggccttcgacgacaccgcgggcgccgccgagtacgtcgccgccaacggcctccgcgacacggccgccatcgcctcctcccgcgccgccgagctgtacggcatggaggtgctcgccgacggcatccaggacgactgcggcaacgtcacccgcttcgtcatgctcgcccgcgagcccatcgtgccccgcaccgaccgccccttcaagaccagcatcgtgttcgcccacgacaaggagggcacctccgtcctcttcaaggtcctctccgccttcgccttccgcgacatcaccctcaccaagatcgagagccgcccgcaccgccaccgccccatccgcctcgtcgacgacgccaacgtcggcaccgccaagcacttcgagtacatgttctacgtcgacttccaggcctccctcgccgagccccgcgcgcagaacgcgctcgccgaggtgcaggagtacacctccttcctccgggtgctcggcagctaccccatggacatgacccccatgaccgccggctcctcctccaccgtcacctccgacgattcctcctccacttgaccgccgccgccgacgaggagacgcgcatcgatcgagtagggacgttggtttcttgtggtcatgtcgtcctactcatcctccccccattgccgggagtaattaattttgcctttcattttttcttattcttcttctcgtcattatacgtgtaattgttccactccagataattaatctgcagtaaatattactagtacaaacgaattcctgttcttgtgttg

SEQ ID NO: 52 - protein, 436 aa, Oryza sativa
MAAASLIKAPVGQNPARMGAGRSSGGGGVVRCSLQGAVVGGRAEWQSSCAVLSSKVAALGAASPHAAAPSFVNGHVAPLVPEQQAAAEDGGAVLDLVPVSSVNGGGVAKNLPQPLRISDLSPAPMHGSQLRVAYQGVPGAYSEKAAGKAYPGCDAIPCDQFEVAFSAVELWIADRAVLPVENSLGGSIHRNYDLLLRHRLHIVGEVQLPVHHCLMALPGVRKECLTRVMSHPQALAQCEHTLTAMGLNVVREAFDDTAGAAEYVAANGLRDTAAIASSRAAELYGMEVLADGIQDDCGNVTRFVMLAREPIVPRTDRPFKTSIVFAHDKEGTSVLFKVLSAFAFRDITLTKIESRPHRHRPIRLVDDANVGTAKHFEYMFYVDFQASLAEPRAQNALAEVQEYTSFLRVLGSYPMDMTPMTAGSSSTVTSDDSSST

SEQ ID NO: 53 - DNA, 1699 bp, Oryza sativa
gctcgttagctaaaactgtgaaaactctccattttccttgcctctctctctccccacccaatcccaacccaccacctctcctctcgccgccgccgccgccgccgccgcgcgccgttctatggcctacacctcctcctccctccacctccacaagcacctcctcctccccaagaccaacccctcctcctcctactcgcggcctccgcctccgtcgttcgtcgccgcggcggccaagatcaacggcgtcaacggacactcctccaagaagtcccccaacggcaaggcgcagatcaacggcgacggcaagaagggggtcaacgggagcgggaggaagaaggcggcggcgcagcacatcaacgggaatgaccggatccatctgtcggtgagcacgggcggcgccgggggccaggatgggttcgggctcagggtggcgttccagggcgcgccgggcgcgtacagcgagttcgcggcgaagacggcgctccccgggtgcgacaccgtgccctgccgcgcgttcgccgacgcgctggcggcggtcgacggcggcgccgtcgaccgcgcgatactgcccgtcgagtccaccatggagggcaccgcgctgcggaactacgacctcctgctgaggcatgacctcgtcgtggtccaggagatcaacctgttcgtgcactactgcctcctcgccatgcccggtgtccgcgccgccgaggtgcgccgggtgattagccaccccatggcgctcgcgcactgcggccgtgcgctggcgcggctcggcgtggacagggagcccgtggaggacaccgccggcgccgtggagatgctgcggtccaaccggatgctcgacaccgccgccatcgccagcccgcgcgccgccgacctctacggcctcgacgtgctcgcgcacggcctccaggacgagtcatggaacgtcacccgcttcctgctcctctccaagccgccctcgccggtgacgctgcccatggacgccgacgccaagaccagcatggtggtcgcccaccggggcggctccatgatggtggtcctcaaggtcctctccgccttctcctcccgcaacatcaacctgaccaagctggaggtcatcaacaacaacgacggcggcggcggcggcggtggcgccgcggcgggccaccccgtgatgatcctcgacacgagcgcccgcggcgcgccgacgctccgcgcgttcccgcacgtcctctacgtcgactgcgagggcgcctctcacgacccgcgcgtcctcgacgccatcaaggagatcgagagattcgcggtgttcgtccgcgttcttggctgctacgccgccgattccaacgtctacgacctgcaatgaggcggccatcgatcgaacacacagctgcggatccacatcaaatcagctagtcttgcaagatttggatcactgatcagttggattagagctctccattccagcactcacttcagtgagctacagatgctatactttgcttgcttgctgctgatgctgctgctgctgcctgctatgcgatttttgtacagatgcaataagaaatctttaccatttctgctgctggcttaggtatgtgcataggtttttaatcctggtattgatgccttctagctgaaggaaagtgtaagatggcacaggattgatgtaattttggtgtgctagatcttgattgatagtgagtattccttaatcccc

SEQ ID NO: 54 - protein, 408 aa, Oryza sativa
MAYTSSSLHLHKHLLLPKTNPSSSYSRPPPPSFVAAAAKINGVNGHSSKKSPNGKAQINGDGKKGVNGSGRKKAAAQHINGNDRIHLSVSTGGAGGQDGFGLRVAFQGAPGAYSEFAAKTALPGCDTVPCRAFADALAAVDGGAVDRAILPVESTMEGTALRNYDLLLRHDLVVVQEINLFVHYCLLAMPGVRAAEVRRVISHPMALAHCGRALARLGVDREPVEDTAGAVEMLRSNRMLDTAAIASPRAADLYGLDVLAHGLQDESWNVTRFLLLSKPPSPVTLPMDADAKTSMVVAHRGGSMMVVLKVLSAFSSRNINLTKLEVINNNDGGGGGGGAAAGHPVMILDTSARGAPTLRAFPHVLYVDCEGASHDPRVLDAIKEIERFAVFVRVLGCYAADSNVYDLQ

SEQ ID NO: 55 - DNA, 1533 bp, populus trichocarpa
atggctgcttctcacatatgggttagccctttaaggcctcattctaatgtaggtgtgtcagatttggggttaaggagatgtgtggatatgaggtgcaattgggatcttgagaggctccctagatgggaatgttgttgtttgagtgttttggctcaaagagctatcactcctgttgaagatgagaagcctttaattcctcaggtggagacttctgaggcaattgatcaggttcaggacactcagtccagggggtttcacaaggatttgaaccttcttcctaaaccgttatctgctacagatattttttcttctcctcgaaatagtgcaaaagtacgggttgcatatcaggggatgccaggtgcctacggtgaggctgctgcgctcaaagcatatccaaagtgtgaaactgtcccatgtgaagagtttgaagctgctttcaaggcagttgaactctggctagtggataaagcagtcctccccattgagagttcagtgggtggcagcatccaccgcaattatgatttactccttcgccataggctgcatatagtaggggaggtgcagatggtagttaaccattgccttttgggattgcctggtgtacagaaggaggaattgaaacgtgttttgagccacccccaggcacttgatcaatgtgacatgatattgaccaaattaggtgttgtcagagttagtactgatgatactgcaggtgctgctctgatggttgcggcaagtggtgaaagagattctggtgtgatcgctagtgatcgtgcagcagaaatctatggccttaacattctattggaaaaaatccaggatgatgatgataatattacccgctttttgatacttgcaagagaacctataattcctggaactgacaggccccataagacaagcattgttttcactctggaagaaggtcctggcatgctgttcaaagcactagcggtgtttgcttcgagggacattaacttgacaaagatagagagtcgcccacaaagaaagcgtccattaagggttgttgatgattctaacaagggaagtgccaggtactttgattaccttttctacattgactttgaagcttccatggcagaacctcgggctcagcacgctatggcacatttgcaggaatttgctagctttcttcgtgttcttggctgctatgcaacagattcagctctttagatggtcatccaacctgaaacagagagattatgattcagcttttgttgctgtacatatgcatgattcagataaatctagagttggagaagagattctctttggcatgatctctcctccattttcaattctctttcataattcttttcactcccccaaaattggggtttcaaggtgtaagttgtaaccattggaagcctaaatagactctgcattcacacggtttctttaaacaattagtgcatgagatgtgtatccctttcactcctcctccaatatttttggacctgcaagatttaataacatggtctttaaagcattgatagttttagccaaaaaaaa

SEQ ID NO: 56 - protein, 397 aa, populus trichocarpa
MAASHIWVSPLRPHSNVGVSDLGLRRCVDMRCNWDLERLPRWECCCLSVLAQRAITPVEDEKPLIPQVETSEAIDQVQDTQSRGFHKDLNLLPKPLSATDIFSSPRNSAKVRVAYQGMPGAYGEAAALKAYPKCETVPCEEFEAAFKAVELWLVDKAVLPIESSVGGSIHRNYDLLLRHRLHIVGEVQMVVNHCLLGLPGVQKEELKRVLSHPQALDQCDMILTKLGVVRVSTDDTAGAALMVAASGERDSGVIASDRAAEIYGLNILLEKIQDDDDNITRFLILAREPIIPGTDRPHKTSIVFTLEEGPGMLFKALAVFASRDINLTKIESRPQRKRPLRVVDDSNKGSARYFDYLFYIDFEASMAEPRAQHAMAHLQEFASFLRVLGCYATDSAL

SEQ ID NO: 57 - DNA, 1693 bp, populus trichocarpa
cagcttgacgttcccttctatagagaactccaagcttaaacctccatagccccggcacaaaatatataaattaatagatatataaacaatccccttaatttctctctaatctccatttccagttcgttttttcaagaattagaaattaactatgcagtcgctatctcctccatctccaatcaagtccctgatccggccacggcctcgggtcatccgagggagtcccaacaaactcgccctccaatgcatctaccgttccgactcggtatctttcccgaacggcgtcggttcgaacagggcagattggcaaagttcctgttctatactagcaagcaaagtggtagctcaacaacagccaaccgataaatctagtagaagtggcgatgttgacggtggagcagatcacgtggctgcagttaacggccacaagacatctatggacttaaaccttgtcccgatagaaaaaaccgcgagctcgaacagcaacagcagcatcaagcctcatcagcctcaaaaaccgttaacgataactgacttatgtcctgcccctatgcatgggtcccatctccgagtcgcgtaccaaggagttccaggcgcgtactctgaagctgctgcaggaaaagcttaccccaactgtgaagcgattccttgtgaccagtttgaggttgcttttcaagctgtcgagctttggatagccgatcgtgctgttttacctgtcgaaaactcactcggtgggtctattcaccggaattatgaccttctcctccgtcaccgactccatatagtaggtgaggttcagttaccggtccaccactgcctccttgccctccctggagtccgaaaggagtatattaaccgcgtcatttcgcacccacaagctcttgctcaatgcgaattgacgctgaccaaactcggtctccaggctgcgcgtgaggccgtggatgatacagcaggtgctgctgagtatattgctgccaacaacctccgtgacacggcggcgattgctagtgcacgcgctgcggagctttatgggatgcaggtgcttgctgatgggatccaagacgattcaagtaatgtcacccgctttgttatgttggcgcgtgagccgattataccgagaacggacaggccgtttaagacgagcatcgtgttcgctcatgataaagggacgagtgtgctgttcaaggtgctttcagcttttgcgtttagaaacatcagtttgacgaagattgaatcaaggccgcataggaaccgtccgattaggttagttgatgatgggaatgtggggacagcaaagcattttgagtacatgttttatattgattttgaagcatccatggctgaggttagagcacagaatgcattggctgaggttcaagaattcacatcattcttgagggtgttagggagctatcccatggacatgacaccttggtgtccatcaagaggagaggatgatgatgatgatgagaaaaacccattttaattatcaagaaaacgtgtaattatttgcacaaaaatggggtttaatttatttttcttttttcttttttcttatctgttgggaatttttaattctacaatgaacaaagaagttgttgataatgacaatgatgatatgattatgataatgatgttgttgttgttgtagtaactattggggtaaatactttaattgcagtagttctgtttt

SEQ ID NO: 58 - protein, 444 aa, populus trichocarpa
MQSLSPPSPIKSLIRPRPRVIRGSPNKLALQCIYRSDSVSFPNGVGSNRADWQSSCSILASKVVAQQQPTDKSSRSGDVDGGADHVAAVNGHKTSMDLNLVPIEKTASSNSNSSIKPHQPQKPLTITDLCPAPMHGSHLRVAYQGVPGAYSEAAAGKAYPNCEAIPCDQFEVAFQAVELWIADRAVLPVENSLGGSIHRNYDLLLRHRLHIVGEVQLPVHHCLLALPGVRKEYINRVISHPQALAQCELTLTKLGLQAAREAVDDTAGAAEYIAANNLRDTAAIASARAAELYGMQVLADGIQDDSSNVTRFVMLAREPIIPRTDRPFKTSIVFAHDKGTSVLFKVLSAFAFRNISLTKIESRPHRNRPIRLVDDGNVGTAKHFEYMFYIDFEASMAEVRAQNALAEVQEFTSFLRVLGSYPMDMTPWCPSRGEDDDDDEKNPF

SEQ ID NO: 59 - DNA, 1469 bp, populus trichocarpa
ttgattttggtttacatttgcatatacattgtgaaagttgcaatctttgttactctttaatggctttaagggctgcttctcccatctggattagccctttaaggcctcactctaaagtgggcgtgtcagatttggggttgaggagatgtgcggatttgaggtgctattgggatcttgagaggcttcctaaatgggaatgttgttgtttaagtgttttggctcaaagagctatcactcctgttgaagatgagaagccttcagctcctcaggtggatacctctagggcaactgatcaggttcaggacactcagtccagagggtttcacaaggatttgaaccttcttcctaaaccgttgtccgctgcagatctttcttcttctcctggaaatggtgcacaagtacgggtagcttatcaggggataccgggtgcatacagtgaggctgctgcgctaaaagcatatccaaagtgcgaaactgttccatgtgatcagtttgaagctgctttcaaggcagttgaactctggttagtggataaagcagtcctccccattgagaattcagtgggtggcagcatccaccgtaattatgatttactccttcgtcataggcttcatatagtaggggaggtgcagatggtagttaaccattgtcttttgggattgcctggtgtaccaaaggaggagttgaaacgtgttttgagccacccccaggcacttgctcaatgtgagatgactttgaccaaattaggtatcatcagagttagtgctgatgattctgcaggtgctgctcagatggttgtggcaaatggggaaagagatactggagcaatcgctagcgctcgtgcagcagacatctatgggcttaacattctattggaaaaaatccaggatgatgatgataatattacccgctttttgatacttgcaagagaacctatgattcctggaagtaacaggccccataagacaagcattgttttcactctcgaagaaggtcctggcatgctgttcaaagctctagcagtgtttgctttgagggacatcaacttgacaaagatagagagtcgccctcaaagaaagcgtccattaagggttgttgatgactctaacaagggaagtgccaggtacttcgattaccttttctacattgactttgacgcttccatggcagaacctcgggcccagcatgctttggcacatttgcaggaatttgcaagatttcttcgtgttcttggctgctatccaacagatgcaactctttaggtggtcactgaacctgcaatgaagagattatgatttagcttttatcaccgtacatatgcatgcttctgataaatctggaggaagagagataactgggatatggtgaagacatgttgaagagagattactgggatatggtgaagacatgttgatgagatgtttgtttctcttcggcatgatctctctttccattttcaattttctctt

SEQ ID NO: 60 - protein, 400 aa, populus trichocarpa
MALRAASPIWISPLRPHSKVGVSDLGLRRCADLRCYWDLERLPKWECCCLSVLAQRAITPVEDEKPSAPQVDTSRATDQVQDTQSRGFHKDLNLLPKPLSAADLSSSPGNGAQVRVAYQGIPGAYSEAAALKAYPKCETVPCDQFEAAFKAVELWLVDKAVLPIENSVGGSIHRNYDLLLRHRLHIVGEVQMVVNHCLLGLPGVPKEELKRVLSHPQALAQCEMTLTKLGIIRVSADDSAGAAQMVVANGERDTGAIASARAADIYGLNILLEKIQDDDDNITRFLILAREPMIPGSNRPHKTSIVFTLEEGPGMLFKALAVFALRDINLTKIESRPQRKRPLRVVDDSNKGSARYFDYLFYIDFDASMAEPRAQHALAHLQEFARFLRVLGCYPTDATL

SEQ ID NO: 61 - DNA, 1644 bp, populus trichocarpa
aagcttaaacctctctaaccccaacagaaaaaaaattaaaaattaatcctaacacccttctttctgtaatctccattcccgattttttagagagaaattcactatgcaatcgctttctactccgtctccgatcaagtccttgatccggccacgtcctcgagtcacccgagcgagtcccaacaaactcgttctcaaatgcatctaccgttccgactcggtttctttcccgaacggtgtcggttcgaaccgagccgattggcaaagttcttgtgctatattggcaagcaaagtggtagctcaacaacagccaatagataaatctattagtgctggcgattctggcggagtagcagatcatgtggccgctgttaacggccacaagacatccgtggacttaaatctagtcccgatagaaaaagccacatcgaactcgaacaacagcagcattaaacctcatcagccacaaaaagcgttaacgataagtgacttatgtcctgcccctatgcacgggtcacaactccgagtcgcgtatcaaggagttcccggcgcgtactctgaagctgctgctggaaaagcctatcccaactgtgaagcgattccttgtgaccagtttgaggttgcttttcaagctgttgagctttggattgccgatcgcgctgttttacctgtcgagaattctctcggtggatccattcatcggaattatgacctcctcctccgtcaccgcctccatatcgttggtgaagttcagttaccggtccaccactgccttcttgcccttcctggagtccggaaggagtatgttaaccgagttatttcgcacccacaagctctcgctcaatgcgaattaactctcaccaaactcggtctccaggcggtgcgtgaagcagtagacgacacagctggtgctgcagagtatatcgcggccaacaacctccgtgacacagctgcgattgcgagtgcacgcgcggcagagctttatgggatgcaggtacttgctgatgggatccaggatgattcaagcaatgtcacgcgctttgtgatgttggcgcgtgaaccgattataccgagaacggacaggccattcaggacaagtattgtgtttgctcatgataaaggaacgagtgtgctgtttaaggtgctttcagcctttgcgtttaggaatataagtttgacgaagattgaatcaaggccgcacaggaaccgtccgattaggctggttgatgatgcaaatgtggggacagcaaagcatttcgagtacatgttttatgttgattttgaagcctcgatggctgaggttagagctcagaatgcattggctgaagttcaagaattcacatctttcctgagggtgttagggagctatccaatggacatgacaccgtggtgtccatcaagagaagacgacaatgacgatggtggtaacgagaaaaacccattttaattacaagtttttttttataaagaaaccctttaaaaagtgttgttatttgcacggaaatgagggtttaatttagtttttctttttttctttgtggggaatttttaattcgaccatgaacaaggaaattgtaatgttattattgatgctgtttgttactgttgaggtaaatacttttattgcaatagttctgttttgtgact

SEQ ID NO: 62 - protein, 446 aa, populus trichocarpa
MQSLSTPSPIKSLIRPRPRVTRASPNKLVLKCIYRSDSVSFPNGVGSNRADWQSSCAILASKVVAQQQPIDKSISAGDSGGVADHVAAVNGHKTSVDLNLVPIEKATSNSNNSSIKPHQPQKALTISDLCPAPMHGSQLRVAYQGVPGAYSEAAAGKAYPNCEAIPCDQFEVAFQAVELWIADRAVLPVENSLGGSIHRNYDLLLRHRLHIVGEVQLPVHHCLLALPGVRKEYVNRVISHPQALAQCELTLTKLGLQAVREAVDDTAGAAEYIAANNLRDTAAIASARAAELYGMQVLADGIQDDSSNVTRFVMLAREPIIPRTDRPFRTSIVFAHDKGTSVLFKVLSAFAFRNISLTKIESRPHRNRPIRLVDDANVGTAKHFEYMFYVDFEASMAEVRAQNALAEVQEFTSFLRVLGSYPMDMTPWCPSREDDNDDGGNEKNPF

SEQ ID NO: 63 - DNA, 1197 bp, populus trichocarpa
atggcagctttcgtgataaaatccctagtaagccaaatcaccgctaaacactcaccgtctgatcatacgtcatcaagaatcgaaatcaaatcagcactgatttctgtgaaacggcgctgcggtagcaaaattccagtcttggcggcttcaattcacagtgaaaacgaccaatcaatcgaggcgcagaagaagaagaacggtaatgctaaaaacattcaatctaatctcttgcaggatgctgaatacgatgtcgcctcgaaagacgcccatccaaggccattatcatcttctcatttatcaaattcagtatccaatgggtctcgccttcgcgttgcttaccagggagttcgtggcgcctacagtgagtcagcagcacaaaaggcgtatccgaattgcgaggctgtcccatgcgagcaatttgatactgcttttgaatcagttgaaagatggcttgtggacagagcagttttaccgattgagaattcgctaggtgggagcattcacagaaattatgacctgttgctccggcacaggctgcatatagtaggagaagtgaagtatgctgttcgccattgcttactagccaatcatggtgttaaagttgaagacttgaagagggttctaagtcatccgcaggctcttgctcaatgtgaaaatacattaacaaagttgggattggtcagagaagcagtggatgataccgctggtgcagcaaagcatgttgcacttcaaaaactagaagacacgggagctgttgctagttctgctgctgcgtcgatctatggattgaatatattagctgaagatattcaggatgattctgataatgtcacacgattccttatactggcaagagaaccaataattccaggcacagataggcctttcaagatccagacaagtatagttttttcattggaggagggtcctggggtgcttttcaaggcccttgctgtttttgctcttcggcaaatcaaccttactaaaattgagagtcgtcccttgcggaagcagcccttgagagcatctgatgatggcaacagtgggcttccaaaatactttgactatctattttatgtggatttcgaagcatcaatggctgacgagaatgctcagaatgccctcaggcatcttaaggagtttgctacgttcttgcgagttctggggagttatccggtgcacactagcatggtgtga

SEQ ID NO: 64 - protein, 398 aa, populus trichocarpa
MAAFVIKSLVSQITAKHSPSDHTSSRIEIKSALISVKRRCGSKIPVLAASIHSENDQSIEAQKKKNGNAKNIQSNLLQDAEYDVASKDAHPRPLSSSHLSNSVSNGSRLRVAYQGVRGAYSESAAQKAYPNCEAVPCEQFDTAFESVERWLVDRAVLPIENSLGGSIHRNYDLLLRHRLHIVGEVKYAVRHCLLANHGVKVEDLKRVLSHPQALAQCENTLTKLGLVREAVDDTAGAAKHVALQKLEDTGAVASSAAASIYGLNILAEDIQDDSDNVTRFLILAREPIIPGTDRPFKIQTSIVFSLEEGPGVLFKALAVFALRQINLTKIESRPLRKQPLRASDDGNSGLPKYFDYLFYVDFEASMADENAQNALRHLKEFATFLRVLGSYPVHTSMV

SEQ ID NO: 65 - DNA, 1903 bp, Picea sitchensis
ggtcgcagatatcgaatttgggaccaagaatggcctccaaactctgtccatcatctcctgctttgggtttctctaactccttattgctacccaagtcgcgtcaatcgacagaaacagcatttcccaagcccaccacgaaactatggagaaattcggcacggtcggcatcacttcatcaatcaggagccgaatggcggattccccgtgcgatgtcatcggcccaatccgcgacccagaaacaggacgagaatcagaacgggagtgttagcctggagtcggggacggttcccaaggacttggtttctctgcccaggcctctgtctgtgacggatcttgctacaccgcccagccatggttctcaggttcgcgtggcttaccagggagttcctggagcatacagtgaggctgcagcattgaaagcttacccacagtgcgaggctgttccttgtgaacaatttgaagcggcattccaggcagttgaactgtggcttgttgataaagcagttttgcctattgaaaattcactcggtggaagcattcaccgtaattatgatttacttcttcgccaccgattgcatatagtcggtgaagtgcagcttgcagttcatcactgccttttgggtcttcctggtgttaagaaggaagagctcaagcgtgttgtaagccatccccaggctctctctcaatgtgagcacacattgagcactttgggggttatacgcgaagcagcagatgatacagctggggctgcccagtttatagctgctaataatcttcgagacacaggggctgttgcaagtgctcgagctgctgagatttatggtctacagattcttgcagatggaatccaggatgattcagacaatgttactcggttcttaatgttggcaagggagcccatcattccaagaattgacagaccattcaagacgagcattgttttcactcttgaagagggacctggagttctttttaaggcacttgcagtgtttgctctaagagatatcaatctaacaaagatagaaagtagacctcagagaaagcggccattgcgggtagtcgatgatagtaatactggtgctgcaaagtattttgactatctcttctacattgactttgaagcttctatggctgatccacgagcacaaaatgccttaggtcacctgcaggaatttgcaacatttatgcgtgttctgggcagttatcctatggacatgacccccacgggtcgattataagtgttgtctgatgaagcctatcatgatttgatgttcacgtaactgagaagtggcttggtcatgagcatggatcttcactatattaatcttttcctgctttgagcatagtgaaacaaatattccagctcccagtatttatcttctgcataggatttgttgagaagatgaaatttgatagggtattttaaaatatgtagatacatacatatgttaagcatttggagctgcattcccctgttactgtacttgcacatgcactgtacaaaactttaagatatcacggatgatatcaaggttgaaattgcatttaagctgggaaatcaacctttggtactatatctggtttgaagtattatttcttctggtctacgccgggtatgttggaagttgccatcataagtaatattaggaacatactcgttttaacttcccggtgatgcctttttgagaagacatacaatgcagccaaatctacctgtatcagaaacccttaaaatagattggtgtgtatctgaactgaagttaatcatagatgcttttatactccatattgggaacaacctactttttctaggatggattcttctctgtttcctattatttttgtacttgaagtggttaattaaaacatttgcaatggtccatgctcaaaaaaaaaaaaaaaa

SEQ ID NO: 66 - protein, 402 aa, Picea sitchensis
MASKLCPSSPALGFSNSLLLPKSRQSTETAFPKPTTKLWRNSARSASLHQSGAEWRIPRAMSSAQSATQKQDENQNGSVSLESGTVPKDLVSLPRPLSVTDLATPPSHGSQVRVAYQGVPGAYSEAAALKAYPQCEAVPCEQFEAAFQAVELWLVDKAVLPIENSLGGSIHRNYDLLLRHRLHIVGEVQLAVHHCLLGLPGVKKEELKRVVSHPQALSQCEHTLSTLGVIREAADDTAGAAQFIAANNLRDTGAVASARAAEIYGLQILADGIQDDSDNVTRFLMLAREPIIPRIDRPFKTSIVFTLEEGPGVLFKALAVFALRDINLTKIESRPQRKRPLRVVDDSNTGAAKYFDYLFYIDFEASMADPRAQNALGHLQEFATFMRVLGSYPMDMTPTGRL

SEQ ID NO: 67 - DNA, 1739 bp, Picea sitchensis
gaacccggaccctcgcctgcatccttataattgtcatcgccctgccagctttggaaaatcttttgctctcagagaagaaaaagaactcaacagcaacagatttgcatttgcaaattgcaggagcagttatttgaagcgacagcgacgattccaagatggtagccggagccagatgcatagcactgaaggtggatacccttcacagcgagaagctgaggtacaaatcttctctctgtagatacaatatttcttcttccatccttaggaccgctgcctgtaaccggcgccacgtttctgtatttgcaaaatacgaagccaataatagaaatggcataagtagtagtaatagtaatggtgatgcaccgatggaagcccgcctgaaagtctgtgaatggcaaagctcctgtgctattctgaatagccagctgcagctccgggcaaaagaagccgaggcggggccggactcgaaggcgctggttcgttcagacagcgccgagtctgatcattccgtctgttctaaggatgtgttacagctgccccggccgctctcgataacggattacagcgcgtttccgaagcacggctcgcagcttcgcgtggcgtaccagggcgtcccgggggcgtacagcgaggcggcagcgggaaaggcctacccgggttgcgagcctgttccctgcgaccaattcgaggcggcattccaggcggtggagctgtgggttgcggatcgggcggtgctgccggtggagaattcactgggcggcagcatccaccgcaactacgaccttctcctccgccaccgccttcatatagtgggcgaggtacagctacgcgtgcaccactgcctcatggcgctcccgggcacgaggaaggaggatctgcggcgcgtgatcagccacccgcaggcacttgcccagtgcgagcacacgatttcgaagctcgtcggcctcaaggtcatccgcgagggcgtggacgacacagccggcgccgcccagatggtggcggagaacgatctccgcgacacggcggctattgccagctcccgggcggctgaaatctacggcatggatatcatcgccgacggaatccaagacgatgccagcaacgtcacccgcttcctgatcctcgcccgggagccaatcattcccggggtggaccgccccttcaagaccagcatagtcttcgcgcaaaacgagggaaccggaattcttttcaaagtccttgccgctttcgccttccgggacattagcctcaccaagatcgagagcaggcctcagagaaaccgccctctccgcgtcgtagacgattccaacctgggaacggccaagtactttgagtatctcttctatatcgatttcgaggcctccttggccgatcccagagctcagaatgcgctcgcggaactgcaggagtttactaattacctcagggtgctggggagttaccccatggacataagtcccccaatcgattcccttgattcttcttaaattaccattcttatttgtaaagaattcggtgaaaataagaggctgggattaactgttgggaattttatatacatgaatatttatatactgttaatgaaattatgaatgccttattgttacattgatgctcagtgctgctgaagagcagctactggtcatatacattatgtctctgtatatgcctctgaaaggccgagcttcacctttcgaattaatgaagtatatgcttttaatcgaaaaaaaaaaaaaaaa

SEQ ID NO: 68 - protein, 443 aa, Picea sitchensis
MVAGARCIALKVDTLHSEKLRYKSSLCRYNISSSILRTAACNRRHVSVFAKYEANNRNGISSSNSNGDAPMEARLKVCEWQSSCAILNSQLQLRAKEAEAGPDSKALVRSDSAESDHSVCSKDVLQLPRPLSITDYSAFPKHGSQLRVAYQGVPGAYSEAAAGKAYPGCEPVPCDQFEAAFQAVELWVADRAVLPVENSLGGSIHRNYDLLLRHRLHIVGEVQLRVHHCLMALPGTRKEDLRRVISHPQALAQCEHTISKLVGLKVIREGVDDTAGAAQMVAENDLRDTAAIASSRAAEIYGMDIIADGIQDDASNVTRFLILAREPIIPGVDRPFKTSIVFAQNEGTGILFKVLAAFAFRDISLTKIESRPQRNRPLRVVDDSNLGTAKYFEYLFYIDFEASLADPRAQNALAELQEFTNYLRVLGSYPMDISPPIDSLDSS

SEQ ID NO: 69 - protein, 286 aa, Arabidopsis thaliana
RISFQGIPGAYSETAALKAFPNCETVPCEQFEAAFQAVEHWLVDKAVLPIENSVGGSIHRNYDLLLRHRLHIVQEVHLPVNHCLLGVPGVKKEDIKCVLSHPQALDQCVNSLNNLGIQRISAKDTATAAQTVSSSGKIDVGAIASVRAANIYGLDILAENIQDDVNNVTRFLILAREPMIPRTDRPYKTSIVFSLEEGPGVLFKALAVFALRSINLSKIESRPQRRRPLRVVDGSNNGSAKYFDYLFYIDFEASMADTRAQHALGHLQEFASFIRILGCYPMDLVR

SEQ ID NO: 70 - protein, 282 aa, Arabidopsis thaliana
RVAYQGVRGAYSESAAEKAYPNCEAVPCEEFDTAFEAVERWLVDRAVLPIENSLGGSIHRNYDLLLRHNLHIVGEVKLAVRHCLLANHGVNIEDLRRVLSHPQALAQCENTLTKLGLVREAVDDTAGAAKQIAFENLNDAAAVASEKAAKIYGLNIVAKDIQDDCDNVTRFLMLAREPIIPGTNRLFKTSIVFSLEEGPGVLFKALAVFALRQINLTKIESRPLRKHPLRASGGLKYFDYLFYVDFEASMADEVAQNALRHLEEFATFLRVLGSYPVDTTML

SEQ ID NO: 71 - protein, 303 aa, Arabidopsis thaliana
RVAYQGVPGAYSEAAAGKAYPNCQAIPCDQFEVAFQAVELWIADRAVLPVENSLGGSIHRNYDLLLRHRLHIVGEVQLPVHHCLIALPGVRKEFLTRVISHPQGLAQCEHTLTKLGLNVAREAVDDTAGAAEFIAANNIRDTAAIASARAAEIYGLEILEDGIQDDASNVTRFVMLAREPIIPRTDRPFKTSIVFAHEKGTCVLFKVLSAFAFRNISLTKIESRPNHNVPIRLVDEANVGTAKHFEYMFYIDFEASMAESRAQNALSEVQEFTSFLRVLGSYPMDMTSWSPSSSSSSSSTFSL

SEQ ID NO: 72 - protein, 299 aa, Arabidopsis thaliana
RVAYQGVPGAYSEAAAGKAYPNCDAIPCDQFDVAFQAVELWIADRAVLPVENSLGGSIHRNYDLLLRHRLHIVGEVQIPVHHCLLALPGVRTDCVSRVISHPQALAQTEHSLDVLTPHAAREAFHDTAAAAEYISANDLHDTAAVASARAAELYNLQILADGIQDDPGNVTRFLMLAREPIIPRTDRPFKTSIVFAAQEHKGTSVLFKVLSAFAFRDISLTKIESRPHHNRPLRVVGDGSFGTSKNFEYMFYVDFEASMAEPRAQNALAEVQEYTSFLRVLGSYPMDMTPWSMTSTEEA

SEQ ID NO: 73 - protein, 299 aa, Arabidopsis thaliana
RVAYQGVPGAYSEAAAGKAYPNSEAIPCDQFDVAFQAVELWIADRAVLPVENSLGGSIHRNYDLLLRHRLHIVGEVQIPVHHCLLALPGVRTDCITRVISHPQALAQTEGSLNKLTPKAAIEAFHDTAAAAEYIAANNLHDTAAVASARAAELYGLQILADGIQDDAGNVTRFLMLARDPIIPRTDRPFKTSIVFAAQEHKGTSVLFKVLSAFAFRNISLTKIESRPHQNCPVRVVGDENVGTSKHFEYTFYVDFEASMAEARAQNALAEVQEYTSFLRVLGSYPMDMTPWSTLPSEDV

SEQ ID NO: 74 - protein, 297 aa, Arabidopsis thaliana
RVAYQGVPGAYSEAAAGKAYPNCQAIPCDQFEVAFQAVELWIADRAVLPVENSLGGSIHRNYDLLLRHRLHIVGEVQLPVHHCLLALPGVRKEFLTRVISHPQGLAQCEHTLTKLGLNVAREAVDDTAGAAEFIASNNLRDTAAIASARAAEIYGLEILEDGIQDDVSNVTRFVMLAREPIIPRTDRPFKTSIVFAHEKGTSVLFKVLSAFAFRDISLTKIESRPNHNRPIRVVDDANVGTAKHFEYMFYVDFEASMAEARAQNALAEVQEFTSFLRVLGSYPMDMTPWSPTSSTSS

SEQ ID NO: 75 - protein, 290 aa, Oryza sativa
KVAYQGCPGAYSEAAAKKAYPNCQTVPCEHFDTAFKAVENWLADRAVLPLENSLGGSIHRNFDLLLRHRLHIVGEVRLAVRHCLLANPGVKIENLKSAMETSHPQALAQCEHTLTEFGIEHREAVDDTAGAAKTVAEQNLQDTGAIASSLAAELYGLNVLAENIQDDKDNVTRFMMLAREPIIPRTDKPFKTSIVFSLEEGPGQLFKALGVFALREINLTKIESRPHKKRPLRITDDSFSTPSKQFDYLFYMDLEASMADPKTQNALGNLKEFATFLRVLGSYPTDVNEA

SEQ ID NO: 76 - protein, 288 aa, Oryza sativa
KVAYQGCPGAYSEAAAKKAYPSCHTVPCEYFETAFQAVENWVADRAVLPLENSLGGSIHRNYDLLLRHRLHIVGEVRLAVRHCLLANRGVKIQNLRSAMSHPQALAQCEQTLTKLGIEHREAVDDTAGAAKLIAEQKLQDTGAVASSLAAQLYGLDILAENIQDDTDNVTRFMMLAREPIIPRTDKPFKTSIVFSLEEGPGQLFKALAVFALRKINLTKMESRPHKKKPLRIADDNCSAPLKHFDYLFYVDLEASMADPNAQNALANLKEFATFLRVLGSYPTDVSEA

SEQ ID NO: 77 - protein, 305 aa, Oryza sativa
RVAYQGVPGAYSEAAAAKAYPSCDAIPCDQFEVAFQAVELWIADRAVLPVENSLGGSIHRNYDLLLRHRLHIVGEVQLPVHHCLLALPGVRRDLLTRVISHPQALAQCELTLNAMGLNVAREAFDDTAAAAEHVAAAGLRDTAAIASSRAAELYGLQVLADGIQDDAGNVTRFVMLAREPIIPRTDRPFKTSIVFAHDREGTSVLFKVLSAFAFRDISLTKIESRPHRHRPIRLVDDANVGTAKHFEYMFYIDFQASMAEVRAQNALSEIQEFTSFLRVLGSYPMDMTPWEYSSSGQTAGDTSSS

SEQ ID NO: 78 - protein, 308 aa, Oryza sativa
RVAYQGVPGAYSEAAAAKAYPSCDAIPCDQFEVAFQAVELWIADRAVLPVENSLGGSIHRNYDLLLRHRLHIVGEVQLPVHHCLLALPGVRRDLLTRVISHPQALAQCELTLNAMGLNVAREAFDDTAAAAEHVAAAGLRDTAAIASSRAAELYGLQVLADGIQDDAGNVTRFVMLAREPIIPRTDRPFKTSIVFAHDREGTSVLFKVLSAFAFRDISLTKIESRPHRHRPIRLVDDANVGTAKHFEYMFYIDFQASMAEVRAQNALSEIQEFTSFLRVLGSYPMDMTPWEYSSSSSPSPAPGGDTSR

SEQ ID NO: 79 - protein, 306 aa, Oryza sativa
RVAYQGVPGAYSEKAAGKAYPGCDAIPCDQFEVAFSAVELWIADRAVLPVENSLGGSIHRNYDLLLRHRLHIVGEVQLPVHHCLMALPGVRKECLTRVMSHPQALAQCEHTLTAMGLNVVREAFDDTAGAAEYVAANGLRDTAAIASSRAAELYGMEVLADGIQDDCGNVTRFVMLAREPIVPRTDRPFKTSIVFAHDKEGTSVLFKVLSAFAFRDITLTKIESRPHRHRPIRLVDDANVGTAKHFEYMFYVDFQASLAEPRAQNALAEVQEYTSFLRVLGSYPMDMTPMTAGSSSTVTSDDSSST

SEQ ID NO: 80 - protein, 306 aa, Oryza sativa
RVAFQGAPGAYSEFAAKTALPGCDTVPCRAFADALAAVDGGAVDRAILPVESTMEGTALRNYDLLLRHDLVVVQEINLFVHYCLLAMPGVRAAEVRRVISHPMALAHCGRALARLGVDREPVEDTAGAVEMLRSNRMLDTAAIASPRAADLYGLDVLAHGLQDESWNVTRFLLLSKPPSPVTLPMDADAKTSMVVAHRGGSMMVVLKVLSAFSSRNINLTKLEVINNNDGGGGGGGAAAGHPVMILDTSARGAPTLRAFPHVLYVDCEGASHDPRVLDAIKEIERFAVFVRVLGCYAADSNVYDLQ

SEQ ID NO: 81 - protein, 220 aa, Oryza sativa
HVAYQGSPGTAIEEMVFKAFPDCIAVPCKKFVAAFEAVDSSLADIVVLPIENSSTGSFHQNYDLLLRHKLHIVQEVQVEIELCLWALPGVQKNDLRTIFSHPEEFAQCEHSLSSLRVIKKNVDHCAAGAEIISMQNLGDAGVIGNAQAAELYGLNIVECNFQDASPNLTRYLVLAKTADIPKEYGQYKGRREYDLLHFPIKCPVLQVSCPDLSDKHRFRS

SEQ ID NO: 82 - protein, 305 aa, populus trichocarpa
RVAYQGVPGAYSEAAAGKAYPNCEAIPCDQFEVAFQAVELWIADRAVLPVENSLGGSIHRNYDLLLRHRLHIVGEVQLPVHHCLLALPGVRKEYINRVISHPQALAQCELTLTKLGLQAAREAVDDTAGAAEYIAANNLRDTAAIASARAAELYGMQVLADGIQDDSSNVTRFVMLAREPIIPRTDRPFKTSIVFAHDKGTSVLFKVLSAFAFRNISLTKIESRPHRNRPIRLVDDGNVGTAKHFEYMFYIDFEASMAEVRAQNALAEVQEFTSFLRVLGSYPMDMTPWCPSRGEDDDDDEKNPF

SEQ ID NO: 83 - protein, 307 aa, populus trichocarpa
RVAYQGVPGAYSEAAAGKAYPNCEAIPCDQFEVAFQAVELWIADRAVLPVENSLGGSIHRNYDLLLRHRLHIVGEVQLPVHHCLLALPGVRKEYVNRVISHPQALAQCELTLTKLGLQAVREAVDDTAGAAEYIAANNLRDTAAIASARAAELYGMQVLADGIQDDSSNVTRFVMLAREPIIPRTDRPFRTSIVFAHDKGTSVLFKVLSAFAFRNISLTKIESRPHRNRPIRLVDDANVGTAKHFEYMFYVDFEASMAEVRAQNALAEVQEFTSFLRVLGSYPMDMTPWCPSREDDNDDGGNEKNPF

SEQ ID NO: 84 - protein, 286 aa, populus trichocarpa
RVAYQGIPGAYSEAAALKAYPKCETVPCDQFEAAFKAVELWLVDKAVLPIENSVGGSIHRNYDLLLRHRLHIVGEVQMVVNHCLLGLPGVPKEELKRVLSHPQALAQCEMTLTKLGIIRVSADDSAGAAQMVVANGERDTGAIASARAADIYGLNILLEKIQDDDDNITRFLILAREPMIPGSNRPHKTSIVFTLEEGPGMLFKALAVFALRDINLTKIESRPQRKRPLRVVDDSNKGSARYFDYLFYIDFDASMAEPRAQHALAHLQEFARFLRVLGCYPTDATL

SEQ ID NO: 85 - protein, 286 aa, populus trichocarpa
RVAYQGMPGAYGEAAALKAYPKCETVPCEEFEAAFKAVELWLVDKAVLPIESSVGGSIHRNYDLLLRHRLHIVGEVQMVVNHCLLGLPGVQKEELKRVLSHPQALDQCDMILTKLGVVRVSTDDTAGAALMVAASGERDSGVIASDRAAEIYGLNILLEKIQDDDDNITRFLILAREPIIPGTDRPHKTSIVFTLEEGPGMLFKALAVFASRDINLTKIESRPQRKRPLRVVDDSNKGSARYFDYLFYIDFEASMAEPRAQHAMAHLQEFASFLRVLGCYATDSAL

SEQ ID NO: 86 - protein, 289 aa, populus trichocarpa
RVAYQGVRGAYSESAAQKAYPNCEAVPCEQFDTAFESVERWLVDRAVLPIENSLGGSIHRNYDLLLRHRLHIVGEVKYAVRHCLLANHGVKVEDLKRVLSHPQALAQCENTLTKLGLVREAVDDTAGAAKHVALQKLEDTGAVASSAAASIYGLNILAEDIQDDSDNVTRFLILAREPIIPGTDRPFKIQTSIVFSLEEGPGVLFKALAVFALRQINLTKIESRPLRKQPLRASDDGNSGLPKYFDYLFYVDFEASMADENAQNALRHLKEFATFLRVLGSYPVHTSMV

SEQ ID NO: 87 - protein, 290 aa, Pinus taeda
RVAYQGVPGAYSETAALKAYSHCDAVPCEQFEAAFQAVELWLVDKAVLPIENSLGGSIHRNYDLLLRHRLHIVGEVKLAVHHCLLGLPGVKKEELKRVVSHPQALSQCEHTLSTLGVIREAVDDTAGAAQFIAANNLRDTGAVASARAAEIYGLQILADGIQDDSDNVTRFLMLAREPIIPRIDRPFKTSIVFTLEEGPGVLFKALAVFALRDINLTKIESRPQRKRPLRVVDDSNTGAAKYFDYLFYIDFEASMADPRAQNALGHLQEFATFMRVLGSYPMDMTPTGRL

SEQ ID NO: 88 - protein, 297 aa, Pinus taeda
RVAYQGVPGAYSEAAAGKAYPGCEPIPCDQFEAAFQAVELWVADRAVLPVENSLGGSIHRNYDLLLRHRLHIVGEVQLPVHHCLLTLPGTRKEDVRRVISHPQALAQCEHTISKLIGHKVIREGVDDTAGAAQMVAENYLRDTAAIASSRAAEIYGMNIIADGIQDDASNVTRFVILAREPIIPRVDRPFKTSIVFAQDEGTGILFKVLAAFAFRDISLTKIESRPQRNRPLRVVDDSNLGNAKYFEYLFYIDFEASMADPRAQNALAELQEFTNFLRVLGSYPMDISPPIDSLDSS

SEQ ID NO: 89 - protein, 300 aa, Pinus taeda
RVAYQGVPGAYSEAAATKAYPSCEPIPCDQFEVAFQAVELWIADRAVLPVENSLGGSIHRNYDLLLRHSLHIVGEVQFPVHHCLLALPGVKPENLRRVISHPQALAQCENTLTLMGLNVAREAFDDTAGAAEFVAANDLRDTASIASARAAEIYGMNILADGIQDDVGNVTRFVMLAREPVIPRTDRPFKTSIVFAHEEGTGVLFKVLSAFAFRNINLTKIESRPQRSKPVRVVDDVNGGTAKHFFEYIFYVDFEASMANPRAQNALAEVQEFTTFLRVLGSYPMDIAPLNRGNSCSNHH

SEQ ID NO: 90 - protein, 297 aa, Pinus taeda
RVAYQGVRGSYCQEAAVRAFQRCDALPCEGEMESAFEALESDAADRAVVPVENSLDGVIGRNYDLILRHPDLHVVGEILLPINHCLLAVRGAGKRRTLKRVISHPQALAHCQRCLGDLGVEVEGVNNAASAARFVAENRIDDTAVIGSAIAGQEYGLEVVEEEMQDDSFNTTRFLIFTKKKPNNNSSAESGLKTTVAFSLKEGTADLCKALSIFAARDIKVTKIESRPLRENPLRMVTNEEQEGSSSKCYFKYVFFVDLEAPVADDHPGQVQMALDSLRHIARFVRVIGNYSVLSVL

SEQ ID NO: 91 - protein, 282 aa, Escherichia coli
RIAFLGPKGSYSHLAARQYAARHFEQFIESGCAKFADIFNQVETGQADYAVVPIENTSSGAINDVYDLLQHTSLSIVGEMTLTIDHCLLVSGTTDLSAINTVYSHPQPFQQCSKFLNRYPHWKIEYTESTSAAMEKVAQAKSPHVAALGSEAGGTLYGLQVLERIEANQRQNFTRFVVLARKAINVSDQVPAKTTLLMATGQQAGALVEALLVLRNHSLIMTRLESRPIHGNPWEEMFYLDIQANLESAEMQKALKELGEITRSMKVLGCYPSENVVPVDPT

SEQ ID NO: 92 - DNA, 1698 bp, Oryza sativa
atgggcggtggagcggcggcgggcaacagccgcggcatcagccaccgaggccttcgtggggagagcaacgacggtggaggacaacgccgccgctgccgacaaggccgagaggcgcggaggcatcgacggccacctctccccatctgctgcaggcggccagaagagaagagaatagaagaaagaaaagaagcgccgccgaccccagcaacatcctctccctttctctctcgccggattggatcgaaacgttggccgccggggaagcagctgggcatggcagggcatctgctcccatttgcccgtggtcatgtacacctccgggagctcgaggaccttgagcagcggcatgagccccttgcgccggagccagttgcagacacggtaggcgtagtgctcgtcggagaggaagagctacagggagggagggaggccgccctttcccgccacccacccctcgccgtcgccgtccgccggatgatggaaactctgcccgcgccgcgccccacccagatccccagatccacggccatccgcccgccgtgcgccacccccttctcccgcgcccactggcagaccacctgcgccgccatcctcgccaccaccactaccaccaacgccaaagcctatggcgccccgcgggtgaacggcgacagcatcaagcccgccctctccgaccacgccgcgccgccgctcgacctggatctgctccccgtctccaacctccctcgcccgctcaccatcaccgacctctcccccgcgcccatgcacggcagccagctgcgcgtcgcttaccagggtgtcccgggggcatacagcgaggccgccgccgccaaggcctacccctcctgcgacgccatcccctgcgaccagttcgaggtggccttccaggccgtcgagctctggatcgccgaccgcgccgtcctccccgtcgagaactccctcggcggcagcatccaccggaactacgacctcctcctccgccaccgcctccacatcgtcggcgaggtccagctccccgtccaccactgcctcctcgcgctcccgggcgtccgcagggacctcctcacccgcgtcatctcccacccgcaggcgctcgcccagtgcgagctcaccctcaacgccatgggcctcaacgtcgcccgcgaggccttcgacgacaccgcggccgccgccgagcacgtcgccgccgcggggctccgcgacaccgccgccatcgcgtcctcccgcgccgccgagctgtacgggctccaggtgctcgcggacggcatccaggacgacgccgggaacgtgacgcggttcgtgatgctggcgcgggagcccatcatcccgcgaacggaccggccattcaagacgagcatcgtgttcgcgcacgacagggagggcacgtcggtgctcttcaaggtgctgtcggccttcgcgttccgggacatcagcctcaccaagatcgagagccgcccacaccgccaccgccccatccggctcgtcgacgacgccaacgtgggcacggccaagcacttcgagtacatgttctacatcgacttccaggcgtccatggcggaggtcagggcgcagaacgcgctgtcggagatacaggagttcacctccttcctccgggtgctcggcagctaccccatggacatgacgccctgggagtattcctcctccggccaaaccgccggcgacaccagcagcagctga

SEQ ID NO: 93 - protein, 565 aa, Oryza sativa
MGGGAAAGNSRGISHRGLRGESNDGGGQRRRCRQGREARRHRRPPLPICCRRPEEKRIEERKEAPPTPATSSPFLSRRIGSKRWPPGKQLGMAGHLLPFARGHVHLRELEDLEQRHEPLAPEPVADTVGVVLVGEEELQGGREAALSRHPPLAVAVRRMMETLPAPRPTQIPRSTAIRPPCATPFSRAHWQTTCAAILATTTTTNAKAYGAPRVNGDSIKPALSDHAAPPLDLDLLPVSNLPRPLTITDLSPAPMHGSQLRVAYQGVPGAYSEAAAAKAYPSCDAIPCDQFEVAFQAVELWIADRAVLPVENSLGGSIHRNYDLLLRHRLHIVGEVQLPVHHCLLALPGVRRDLLTRVISHPQALAQCELTLNAMGLNVAREAFDDTAAAAEHVAAAGLRDTAAIASSRAAELYGLQVLADGIQDDAGNVTRFVMLAREPIIPRTDRPFKTSIVFAHDREGTSVLFKVLSAFAFRDISLTKIESRPHRHRPIRLVDDANVGTAKHFEYMFYIDFQASMAEVRAQNALSEIQEFTSFLRVLGSYPMDMTPWEYSSSGQTAGDTSSS

SEQ ID NO: 94 - DNA, 500 bp, populus trichocarpa
atggctgcttctcacatatgggttagccctttaaggcctcattctaatgtaggtgtgtcagatttggggttaaggagatgtgtggatatgaggtgcaattgggatcttgagaggctccctagatgggaatgttgttgtttgagtgttttggctcaaagagctatcactcctgttgaagatgagaagcctttaattcctcaggtggagacttctgaggcaattgatcaggttcaggacactcagtccagggggtttcacaaggatttgaaccttcttcctaaaccgttatctgctacagatattttttcttctcctcgaaatagtgcaaaagtacgggttgcatatcaggggatgccaggtgcctacggtgaggctgctgcgctcaaagcatatccaaagtgtgaaactgtcccatgtgaagagtttgaagctgctttcaaggcagttgaactctggctagtggataaagcagtcctccccattgagagttcagtgggtgg

SEQ ID NO: 95 - DNA, 500 bp, populus trichocarpa
atgcagtcgctatctcctccatctccaatcaagtccctgatccggccacggcctcgggtcatccgagggagtcccaacaaactcgccctccaatgcatctaccgttccgactcggtatctttcccgaacggcgtcggttcgaacagggcagattggcaaagttcctgttctatactagcaagcaaagtggtagctcaacaacagccaaccgataaatctagtagaagtggcgatgttgacggtggagcagatcacgtggctgcagttaacggccacaagacatctatggacttaaaccttgtcccgatagaaaaaaccgcgagctcgaacagcaacagcagcatcaagcctcatcagcctcaaaaaccgttaacgataactgacttatgtcctgcccctatgcatgggtcccatctccgagtcgcgtaccaaggagttccaggcgcgtactctgaagctgctgcaggaaaagcttaccccaactgtgaagcgattccttg

SEQ ID NO: 96 - DNA, 500 bp, populus trichocarpa
atggctttaagggctgcttctcccatctggattagccctttaaggcctcactctaaagtgggcgtgtcagatttggggttgaggagatgtgcggatttgaggtgctattgggatcttgagaggcttcctaaatgggaatgttgttgtttaagtgttttggctcaaagagctatcactcctgttgaagatgagaagccttcagctcctcaggtggatacctctagggcaactgatcaggttcaggacactcagtccagagggtttcacaaggatttgaaccttcttcctaaaccgttgtccgctgcagatctttcttcttctcctggaaatggtgcacaagtacgggtagcttatcaggggataccgggtgcatacagtgaggctgctgcgctaaaagcatatccaaagtgcgaaactgttccatgtgatcagtttgaagctgctttcaaggcagttgaactctggttagtggataaagcagtcctccccattgagaattc

SEQ ID NO: 97 - DNA, 1971 bp, Picea sitchensis
gagctcgcgtttctgcattcaagggtctgcagccgagcatcacacaacacaatccaattcattcaccgccattcgggcaatatacccaatccattttcagatcatattgaatttgatagatcccagaaatgcagagcgtatcagtgagcttgcaagcggcctctcatggcgggctggagggcatgagagaatttaattttttcatgggtacccgtgtgcgtaaggatggatttagaattaaaatgaggaaaaggggggccggcagcattgaagccgtgtatacgatgagatcgggaacggagaacctgcaacaatcgagcaacgctacttcaacacagtggcagacctcctgcgctattttgtcgagcaacgttgtctcgcagcagcaagagacggagaaatctgccgaccacggcctggttgctgtcaatggccacgccaaatcggagtccatgatcccccgggacctcgacaacttgccaagaccgttgtcgttgatagacctgtcgcctccgcccatgcacggctccccgcttcgcgtggcttatcagggtgtgccaggtgcctacagcgaggccgcggctaagaaggcgtaccccaactcagaggccattccctgcgaccagttcgaggtggctttccaggcggtggagctctggatagccgatcgggcagttcttcctgtggagaattcgctgggaggaagcatccacagaaattacgatctcctactccgtcacaggcttcacattgttggggaggttcagttgcctgttcatcattgcctcctggcgcttccgggcgtgagaaaggagtatctgaatcgggtgatcagccacccgcaggcgctttcccagtgcgagctcacgctcaccaagctcggcctcaatgtagcccgcgaggctttcgatgacactgccggtgccgccgagttcattgccctcaacaacctccgcgacacggcggccatagccagctctcgggcagccgagctttacggtatgaccgtcctggccgatggcatccaggacgacagcaacaacgtgactcgcttcgttatgctggcgagggaacccatcattccaagaaccgacaggcccttcaagaccagcattgtgtttgctcaagataagggtacctctgtcttgttcaaggtcttgtcggcctttgctttcaggaatataagcctgaccaagattgagagcaggcctcacaggaaccagccactccgtgtggtcgatgacggtaatgttattggcactgccaagcacttcgagtacatgttctatgtcgattttgaggcctccatggccgacccaagagcccagaatgccctttcagaggtccaagaattcacctccttccttagggttctgggcagctaccccatggacatgacccccttgaataataactcatcgtcatcttcatcctcatccccatcctcttgttgataaatcaatgatattaaataccccttctctgggctttctgatcaggaaattctagttcttgtttttcttgatcttgaagttctattttttccatatcaataaacaggttcctggttcatggttcttcaaggagagagcagagcagctcacaatgatttgggcaatgagaatgaaatgatttatataatattgtcgaatatttatggcagatgaaggcgtgatttgaagcttcttgttgttatagtttcgatggaaccttaagattttcctctgtaaattcaatccgcccccttcaaaggagattgcagaaagaaagctgtaaatgtaaatgcagtatcctgtgtccctttgtggcagagtagtatttggttgcccttcctatagtattccatttgtaattttgtattacctatctcagtcatggcatcctggtcatgtattttgtttgattattcctgtaaggttacaaaaacttctttatcgaaattgaggaaaatttacaaaaaaaaaaaaaaaa

SEQ ID NO: 98 - protein, 441 aa, Picea sitchensis
MQSVSVSLQAASHGGLEGMREFNFFMGTRVRKDGFRIKMRKRGAGSIEAVYTMRSGTENLQQSSNATSTQWQTSCAILSSNVVSQQQETEKSADHGLVAVNGHAKSESMIPRDLDNLPRPLSLIDLSPPPMHGSPLRVAYQGVPGAYSEAAAKKAYPNSEAIPCDQFEVAFQAVELWIADRAVLPVENSLGGSIHRNYDLLLRHRLHIVGEVQLPVHHCLLALPGVRKEYLNRVISHPQALSQCELTLTKLGLNVAREAFDDTAGAAEFIALNNLRDTAAIASSRAAELYGMTVLADGIQDDSNNVTRFVMLAREPIIPRTDRPFKTSIVFAQDKGTSVLFKVLSAFAFRNISLTKIESRPHRNQPLRVVDDGNVIGTAKHFEYMFYVDFEASMADPRAQNALSEVQEFTSFLRVLGSYPMDMTPLNNNSSSSSSSSPSSC

SEQ ID NO: 99 - DNA, 1700 bp, Vitis vinifera
gaacaccaagctacaacccccctctcctctcttctctttttctttttcatttggtttttcccctggtcatcagagattcagaatatgcaggctctatctccatctcctaatctcaaatccttaatcaggacgaggccgctcgttcccactcaccgcgtcgctccacaccgactcgtggttcaatgtgtttacaaatccgactactctaacttctccggcggcgtcgggttcagccgagctgattggcaaagctcttgcgccatcttagctagcaaggttgtgtcgcagcagcaagacaccgagaagagcggcaacgccgatctgaccgctgttaatggccacaagacactagatctcgtccccatcgagaatctgcccaagccgctcaccatcaccgacctctcacccgctccgatgcacggctcggagctccgtgtggcttaccagggtgttcccggcgcgtacagcgaggccgccgccggaaaagcgtatccgaactgcgaggcgataccgtgtgaccaattcgaggtggcgtttcaggctgtggagctgtggatcgccgatcgagctgtgctgccggtggagaactcgctcggaggcagcattcaccggaattatgacctcctgcttcgccaccgcctccacatcgtgggcgaggtccaacttccggtgcaccactgcctgttagccttaccgggagtgcgaaaagagtacctgactcgcgtgataagccatcctcaagccctggcccagtgcgagcacacgctcaccaaactcggcctcaacgtcgctcgcgaggccgtggacgacaccgccggagccgcggagtacgtagcgttgaacaacctccgagatacggcggcgatcgcgagcgcccgagcggcggatctgtacggactccagatattggcggacggcatccaagacgactcgagcaacgtgactcggttcgtgatgcttgcgcgcgagccaatcatccctcgcaccgaccggccattcaagaccagcatcgtattcgcacacgacaagggaacgtccgtactgttcaaggttctgtcagcgttcgccttcagaaacataagcttgacgaagatcgagagcagacctcaccgaaaccgtccgatcagattggtggacgacgcgaacgtgggaacggcaaagcacttcgagtacatgttctacgtggatttcgaggcatccatggcagaagtcagagcccagaacgcactggcggaggttcaagagttcacatctttcttgagggtgttggggagctatcctatggacatgacgccttggtgcccttctgggggagattaagacttcagatttaagttaaaagttaaaaaaaaaaaacacacttctcctggtagagccagagattggtagagccagagatttgttatttgcataacccacccatccatccatccatccatccgcccctacaagaaaaggcaaaaagtccatttaaattccctttttattattttcttttttggattttaaattcatcaagggattttaaacaacaagcaacattgttgttgggtaaatatgtagtttgattatccatcacatttgtggttattactctgtaacaatgtgaataccattcccagccaatctttgtatttgatgcttgggacattgagcaaaagcaaaagcaacaccaacaaatagaatgtctattttttatt

SEQ ID NO: 100 - protein, 411 aa, Vitis vinifera
MQALSPSPNLKSLIRTRPLVPTHRVAPHRLVVQCVYKSDYSNFSGGVGFSRADWQSSCAILASKVVSQQQDTEKSGNADLTAVNGHKTLDLVPIENLPKPLTITDLSPAPMHGSELRVAYQGVPGAYSEAAAGKAYPNCEAIPCDQFEVAFQAVELWIADRAVLPVENSLGGSIHRNYDLLLRHRLHIVGEVQLPVHHCLLALPGVRKEYLTRVISHPQALAQCEHTLTKLGLNVAREAVDDTAGAAEYVALNNLRDTAAIASARAADLYGLQILADGIQDDSSNVTRFVMLAREPIIPRTDRPFKTSIVFAHDKGTSVLFKVLSAFAFRNISLTKIESRPHRNRPIRLVDDANVGTAKHFEYMFYVDFEASMAEVRAQNALAEVQEFTSFLRVLGSYPMDMTPWCPSGGD

SEQ ID NO: 101 - DNA, 1526 bp, Vitis vinifera
atggcggcttccttctcacgatctcccatcacgccccttcaccgccaaaacaactgcaaattcccgcccttggatccaatggcgagagtgcacattagtactcctggaatatcttcgaagcggcgctgtaatatctctctggcctatgtgcccaacgacaatgatgagaacgcgaagtctcgagaattgcagagaagtttggaagactttccctctgaaaccctttcaaaagggcctttgtcttcgacccatttgtctaataagctctctgataggtctcgccttcgggtggcataccagggagttcatggtgcctacagcgagtctgctgcggagaaggcctacccgaactgtcaagctgttccttgtgagcaatttgaaactgcttttgaggctgttgaaagttggcttgttgatagagcagttttgccaattgagaattctcttggggggagcattcacaggaactatgaccttttacttcgccacaggttgcatatagttggggaagtaaagtttgcagtccgacattgcttactagccaatcatggtgttaaagttgaagacctcaagagggttcttagccattctcaggctcttgctcagtgtgagaacacactaacaaagttgggattggtcagagaagcagtggatgatactgctggtgcagctaagtttattgctttccacaaactgaaagatgtgggagccgttgcaagctctgctgctgcaaggatctatggtttgaaaatacttgctcaagacattcaggatgattcttataatgttactcgatttcttatgctggcaagggaacccataattcctggcacagacaggccattcaagacaagtattgttttctcattggaggaaggtcctggggtgcttttcaaggcacttgctgtatttgccctaaggcagatcaaccttacaaaaattgaaagtcgtccattgaggaatcagcctttgcgagcatccaatgataccaataatgggagtccaaaatactttgattatcttttctatgtggattttgaagcatcaatggccgaccaaaattcacagaatgccctcaggcacctgaaggagtttgctacattcctgagagtgctagggagttaccctgtggacactagcatgatataactaattattaagcctggattcagtgcttgagttcagttatggcatgttacattgtaaagatagtaattactgatgtatcaagaaatgaagactcgtgttatatagatttgtttttctccatcaatcgtttcttatagatccaacacaaccatgaaatacagaatctgggctaacatctaatctagttatgcaatttgtctttccccgttttccttatattttcaatgtaattactggacgtagttgtgacacattaaatgagcttaaggttattttataatatttgtgtatctgtttttactttgattgatcttgtacatgatttgaaatgagaagtttaatgctaatcagtggccgctcaggcacggttttgcacc

SEQ ID NO: 102 - protein, 382 aa, Vitis vinifera
MAASFSRSPITPLHRQNNCKFPPLDPMARVHISTPGISSKRRCNISLAYVPNDNDENAKSRELQRSLEDFPSETLSKGPLSSTHLSNKLSDRSRLRVAYQGVHGAYSESAAEKAYPNCQAVPCEQFETAFEAVESWLVDRAVLPIENSLGGSIHRNYDLLLRHRLHIVGEVKFAVRHCLLANHGVKVEDLKRVLSHSQALAQCENTLTKLGLVREAVDDTAGAAKFIAFHKLKDVGAVASSAAARIYGLKILAQDIQDDSYNVTRFLMLAREPIIPGTDRPFKTSIVFSLEEGPGVLFKALAVFALRQINLTKIESRPLRNQPLRASNDTNNGSPKYFDYLFYVDFEASMADQNSQNALRHLKEFATFLRVLGSYPVDTSMI

SEQ ID NO: 103 - DNA, 1245 bp, Vitis vinifera
atggctttgaagactggactaatctggggttctgcaactcctccgcatcctcacttgggtctggcggatttgggtggtaggcgagctgggcgtgctctgaatttgaggtttgattttgagaggtttcggaaatgggagtgtttggctgttttgggtcagcgagctacaatccctgttgaagatgagaagccactgaggcctggggttgagtctcctggtggggctgatgaagccaaggagactgagcccagggcttttcacagggatttgaactctcttccaagaccattatctgctacagatccttcatcgtctcctagcaatggtgggaaggtgcgggttgcttatcagggggcaccaggtgcatacagtgaggaagctgcgatgaaagcatacccaaagtgtgaggctgttccttgtgatgatttcgaagctgcattcaaggcagttgaattgtggttggtggagaaagcagttctcccaattgagaattctgtaggtggaagcatccatcgtaattatgatcttctccttggccacaggctgcacatagtgggggaggtgcagatggttgttaaccattgcctattgggattgcctggtgtaagaaaggatgagctaaaacgtgttttaagtcatcctcaggcatttgctcagtgtgatatgacattaaatgagttaggcctcctcagaattagcacagaggatactgccggtgctgctcagattgtcgcttctgatggcctgaaaaatactggagcaattgccagtgcccgagctgcagtaatctatgggcttaacattcttgaagagaaaatccaggatgattgtgacaatattacccggtttttgatacttgctagagaacctataatcccaggacttgagaggccttataagacaagcattgtcttcagtcttgatgaaggccctggggtactcttcaaagccctggctgtgtttgctctgagagatattagtttgtcaaagattgagagccgtccacaaagaaagcgtccactgaggatcgttgatgactctaacaaaggcagtgctaagtattttgattaccttttctatatcgactttgaagcttctatggcagagcctcgtgctcaatatgctctgggtcacctgcaggaatttgcaagatttctgagggtcctcggttgctatccaatggatcaaacaccttacgaagcaacttcagttgataacgcggggcctgctgctggaaacagttaa

SEQ ID NO: 104 - protein, 414 aa, Vitis vinifera
MALKTGLIWGSATPPHPHLGLADLGGRRAGRALNLRFDFERFRKWECLAVLGQRATIPVEDEKPLRPGVESPGGADEAKETEPRAFHRDLNSLPRPLSATDPSSSPSNGGKVRVAYQGAPGAYSEEAAMKAYPKCEAVPCDDFEAAFKAVELWLVEKAVLPIENSVGGSIHRNYDLLLGHRLHIVGEVQMVVNHCLLGLPGVRKDELKRVLSHPQAFAQCDMTLNELGLLRISTEDTAGAAQIVASDGLKNTGAIASARAAVIYGLNILEEKIQDDCDNITRFLILAREPIIPGLERPYKTSIVFSLDEGPGVLFKALAVFALRDISLSKIESRPQRKRPLRIVDDSNKGSAKYFDYLFYIDFEASMAEPRAQYALGHLQEFARFLRVLGCYPMDQTPYEATSVDNAGPAAGNS

SEQ ID NO: 105 - DNA, 1824 bp, Vitis vinifera
tgacccagatcaatccaaccacggtgaggcttcgaagggaaaacaattcagcttaacgtctccatccgttacaacttccaaatcgcagagcttgtcaaagaagtggaagaagtctttacagataccccaaaacgtgttcacctaacctggtatcaggtgtttgatcaaagtcgaagcacagcattttgtttttcatgtttttatggctttgaagtttagttctgtgttgggtggtgctgtttcccagttgggttgggaagatttgggctccaagccttctggggtggtctgcaattggggtaagggcactgcggagactcccagtgtggttttttcgggcttgtcgactcaccgggggataaagtcgttggatgatgggaacccgtcaaatccgggaactgaattgcaggggattgttgataaggtggataacaatgactccgggagaatacacagggatctggcatcatttccaaaaccattatctgtcactgatatttctgctgctcccaaggatggtacaaaggtgcgcatttcatataagggtgtgccaggcgcatatagtgaggacgctgccctcaaagcttaccctcattgtgaaactgttccttgtgatgagtttgaagatgcattcaaggctgttgaattgtggttggctgaaaaagcagttcttcccattgagaattctttaggtggaagcatccatcgtaactatgatttactccttcgtcataggctgcacattgtgggtgaggtgcagttggctgttaacctctgcctcctagctataccaggggtgggaattgatcagctaagacgtgttctaagccatcctcaggcactcgcccagagtgatatcatcctgagtaagttaggtgtttccagagaaaatgttgatgattcagctggtgctgctcagtatgtagcctcacatggcctgagggatgctggtgttgttgcaagtgctcgagcagcagaaatctatgggcttaacatacttgcagaaagaatccaggatgactttgataacattactcgttttctggtccttgcaagggatcctataattccaagaaccaataagctctttaagacaagcattgtgtttactttggaagaaggccctggagtattatttaaagccttggcagtctttgctttgagagacataaatttgacaaagattgaaagccggccacagaggaagaaaccattaagggtagttgatgactccaacactggaagtgccaagtattttgactacctcttttatattgattttgaggcttctatggctgagccccgtgcacagacagctttggcacatcttcaggagtttgccacatttctccgtgtacttggttgctatcctatggattcctttccttagcaatcatgatcttgatgccagtcctcccataataaaacttatcctgctcaaccccagtagggaatggagttcatccagaattttgggatggcctgctgtccaaagcttgtatacatactattttatttgatgatgctggatcttggatcctgcaggtcactgtatttggttgtcaaagacacttcaaacatggattttgggacccattgggttgtacgttataagataaaaagattctagcttgcttgatacagtattgggcaattgttctagtacagacaggcacaactgcccgcacattagactggattgctgttattgagaactcaaattcctcatcagggttatctacagattccccattcatgtgtggattgcaggtctcagcctcttctgtttccataaatataaattacccttctgtggcttttgaa

SEQ ID NO: 106 - protein, 395 aa, Vitis vinifera
MALKFSSVLGGAVSQLGWEDLGSKPSGVVCNWGKGTAETPSVVFSGLSTHRGIKSLDDGNPSNPGTELQGIVDKVDNNDSGRIHRDLASFPKPLSVTDISAAPKDGTKVRISYKGVPGAYSEDAALKAYPHCETVPCDEFEDAFKAVELWLAEKAVLPIENSLGGSIHRNYDLLLRHRLHIVGEVQLAVNLCLLAIPGVGIDQLRRVLSHPQALAQSDIILSKLGVSRENVDDSAGAAQYVASHGLRDAGVVASARAAEIYGLNILAERIQDDFDNITRFLVLARDPIIPRTNKLFKTSIVFTLEEGPGVLFKALAVFALRDINLTKIESRPQRKKPLRVVDDSNTGSAKYFDYLFYIDFEASMAEPRAQTALAHLQEFATFLRVLGCYPMDSFP

SEQ ID NO: 107 - protein, 295 aa, Vitis vinifera
RVAYQGVPGAYSEAAAGKAYPNCEAIPCDQFEVAFQAVELWIADRAVLPVENSLGGSIHRNYDLLLRHRLHIVGEVQLPVHHCLLALPGVRKEYLTRVISHPQALAQCEHTLTKLGLNVAREAVDDTAGAAEYVALNNLRDTAAIASARAADLYGLQILADGIQDDSSNVTRFVMLAREPIIPRTDRPFKTSIVFAHDKGTSVLFKVLSAFAFRNISLTKIESRPHRNRPIRLVDDANVGTAKHFEYMFYVDFEASMAEVRAQNALAEVQEFTSFLRVLGSYPMDMTPWCPSGGD

SEQ ID NO: 108 - protein, 287 aa, Vitis vinifera
RVAYQGVHGAYSESAAEKAYPNCQAVPCEQFETAFEAVESWLVDRAVLPIENSLGGSIHRNYDLLLRHRLHIVGEVKFAVRHCLLANHGVKVEDLKRVLSHSQALAQCENTLTKLGLVREAVDDTAGAAKFIAFHKLKDVGAVASSAAARIYGLKILAQDIQDDSYNVTRFLMLAREPIIPGTDRPFKTSIVFSLEEGPGVLFKALAVFALRQINLTKIESRPLRNQPLRASNDTNNGSPKYFDYLFYVDFEASMADQNSQNALRHLKEFATFLRVLGSYPVDTSMI

SEQ ID NO: 109 - protein, 302 aa, Vitis vinifera
RVAYQGAPGAYSEEAAMKAYPKCEAVPCDDFEAAFKAVELWLVEKAVLPIENSVGGSIHRNYDLLLGHRLHIVGEVQMVVNHCLLGLPGVRKDELKRVLSHPQAFAQCDMTLNELGLLRISTEDTAGAAQIVASDGLKNTGAIASARAAVIYGLNILEEKIQDDCDNITRFLILAREPIIPGLERPYKTSIVFSLDEGPGVLFKALAVFALRDISLSKIESRPQRKRPLRIVDDSNKGSAKYFDYLFYIDFEASMAEPRAQYALGHLQEFARFLRVLGCYPMDQTPYEATSVDNAGPAAGNS

SEQ ID NO: 110 - protein, 286 aa, Vitis vinifera
RISYKGVPGAYSEDAALKAYPHCETVPCDEFEDAFKAVELWLAEKAVLPIENSLGGSIHRNYDLLLRHRLHIVGEVQLAVNLCLLAIPGVGIDQLRRVLSHPQALAQSDIILSKLGVSRENVDDSAGAAQYVASHGLRDAGVVASARAAEIYGLNILAERIQDDFDNITRFLVLARDPIIPRTNKLFKTSIVFTLEEGPGVLFKALAVFALRDINLTKIESRPQRKKPLRVVDDSNTGSAKYFDYLFYIDFEASMAEPRAQTALAHLQEFATFLRVLGCYPMDSFP

(19) United States
(12) Patent Application Publication
(10) Pub. No.: US 2013/0318653 A1
(43) Pub. Date: Nov. 28, 2013

(54) AROGENATE DEHYDRATASES AND LIGNIFICATION

(71) Applicants: Norman G. Lewis, Pullman, WA (US); Laurence B. Davin, Pullman, WA (US); Oliver R.A. Corea, Pullman, WA (US); Sung-Jin Kim, Pullman, WA (US)

(72) Inventors: Norman G. Lewis, Pullman, WA (US); Laurence B. Davin, Pullman, WA (US); Oliver R.A. Corea, Pullman, WA (US); Sung-Jin Kim, Pullman, WA (US)

(73) Assignee: Washington State University, Pullman, WA (US)

(21) Appl. No.: 13/884,472

(22) PCT Filed: Nov. 9, 2011
(86) PCT No.: PCT/US2011/060043

§ 371 Date: May 9, 2013

Related U.S. Application Data
(60) Provisional application No. 61/411,872, filed on Nov. 9, 2010.

(51) Int. Cl. C12N 15/82

(52) U.S. Cl. CPC C12N 15/8255
USPC 800/278; 435/468; 800/298; 435/419; 800/319; 44/605; 426/615; 426/635

(57) ABSTRACT

Provided are methods for decreasing carbon flow into lignin in plants, comprising reducing or eliminating, using mutagenesis and/or recombinant means, expression and/or activity of at least one chloroplast-localized arogenate dehydratase (ADT) sufficient to reduce phenylalanine (Phe) availability for metabolism into Phe-derived phenylpropanoids, wherein the amount, level or distribution of lignin is reduced relative to control plants. In particular aspects, the plant has a plurality of chloroplast-localized ADTs, and reducing or eliminating comprises reducing or eliminating expression and/or activity of at least two of the plurality of ADTs. Also provided are recombinant plants or parts or cells thereof, comprising at least one mutation, genetic alteration or transgene that reduces or eliminates the expression and/or activity of at least one chloroplast-localized ADT, wherein the amount, level or distribution of lignin is reduced relative to normal. Further provided are reduced lignin plant products.

### CROSS-REFERENCE TO RELATED APPLICATIONS

This application claims the benefit of priority to U.S. Provisional Patent Application No. 61/411,872 filed 9 Nov. 2010 and entitled “AROGENATE DEHYDRATASES AND LIGNIFICATION,” which is incorporated herein by reference in its entirety.

### STATEMENT REGARDING FEDERALLY-SPONSORED RESEARCH

This work was supported at least in part by grants from the Chemical Sciences, Geosciences and Biosciences Division, Office of Basic Energy Sciences (DE-FG-0397ER20259), Office of Science, U.S. Department of Energy, from the BioEnergy Science Center, the U.S. Department of Energy Bioenergy Research Center supported by the Office of Biological and Environmental Research in the DOE Office of Science, from the United States Department of Agriculture (Agricultural Plant Biochemistry #2006-03339), and the U.S. government has certain rights in the invention.

### FIELD OF THE INVENTION

Aspects of the present invention relate generally to lignin production in plants, and more particularly to methods for decreasing carbon flow into lignin in plants, comprising reducing or eliminating expression and/or activity of at least one, and preferably more than one, chloroplast-localized arogenate dehydratase (ADT) sufficient to reduce phenylalanine (Phe) availability for metabolism into Phe-derived phenylpropanoids, wherein the amount, level or distribution of lignin is reduced relative to control plants. Additional aspects relate to recombinant plants or parts thereof, comprising at least one mutation, genetic alteration or transgene that reduces or eliminates the expression and/or activity of at least one, and preferably more than one, chloroplast-localized ADT, wherein the amount, level or distribution of lignin is reduced relative to normal. Further aspects relate to reduced lignin plant products.

### SEQUENCE LISTING

A Sequence Listing containing 110 sequences was filed as part of this application, and is incorporated herein by reference in its entirety.

### BACKGROUND

The final step of Phe biosynthesis, catalyzed by arogenate dehydratase (ADT) in planta (1-3), is potentially a major regulatory point due to both its pivotal position at the branch-point of Tyr and Phe biosynthesis (see FIG. 2), and as a linkage point between plastid/chloroplast localized shikimate-chorismate and cytosolic/membrane associated phenylpropanoid metabolic networks. Together, these pathways comprise some of the most metabolically intensive networks in vascular plants. Indeed, depending upon the species, up to 50% of captured photosynthetic carbon can be in the form of Phe-derived phenylpropanoids (4, 5). For examples, downstream phenylpropanoid-derived products can have important but distinct physiological functions in planta, as fragrances/flavors, defense molecules, UV protectants, pigments, structural biopolymers, and so forth, e.g. allyl/propenyl phenols, lignans (6), flavonoids, (proantho)cyanidins, phytoalexins (e.g. isoflavones), lignins (7) and suberins (8, 9). The broad physiological functions of phenylpropanoid-derived metabolites thus translate into a diverse and ever-changing demand for the pathway intermediate Phe, i.e. in addition to its utilization for protein synthesis and other metabolic pathways.

Curiously, the question of pivotal metabolic networks upstream of Phe profoundly altering carbon flux/allocation into phenylpropanoid metabolism versus protein synthesis etc. had essentially not been addressed before. Instead, previous biotechnological manipulations targeted the presumed “entry” point to the phenylpropanoid pathway, phenylalanine ammonia lyase (PAL), as well as various downstream monolignol pathway steps (see Anterola and Lewis (10) and Davin et al. (7) for a discussion). Such approaches, however, did not take into much consideration the potential seamless integration of related upstream, but differentially localized, metabolic networks associated with same, and transcriptional regulation thereof. Applicants considered that this was relevant since, in previous metabolic flux studies leading to monolignols in loblolly pine (Pinus taeda), it was established that factors apparently affecting Phe availability helped control carbon flux into phenylpropanoid metabolism (11, 12), rather than PAL having a central rate-limiting role as had often been reported due to its “entry point” position to phenylpropanoids.

According to particular aspects of the present invention, the ADT family was considered by Applicants as a potentially promising candidate for involvement in regulating the previously documented changes in Phe availability in plants, due to its branch-point position in the shikimate-chorismate pathway, and its sensitivity to feedback inhibition by Phe. Indeed, Applicants had previously characterized all six ADT isoenzymes from Arabidopsis thaliana, and provided molecular and biochemical evidence supporting the arogenate route as the major mode of Phe biosynthesis (FIG. 2) (1). All of the ADT isoforms in Arabidopsis are targeted to chloroplasts/plastids, and are apparently expressed in stems, leaves, roots, flowers, siliques and seeds (1, 13). Specifically, three isoenzymes, ADT3, ADT4 and ADT5 demonstrated exclusive substrate preference for arogenate, while isoenzymes ADT1, ADT2 and ADT6 displayed instead a strong substrate preference for arogenate, but also had limited ability to utilize prephenate (1) (in a previous investigation, genes encoding six ADT isoforms were established to be present in the Arabidopsis genome, with these forming three putative subgroups based on phylogenetic analysis (1): I (ADT1, At1g11790); II (ADT2, At3g07630); and III (ADT3, At2g27820; ADT4, At3g44720; ADT5, At5g22630; ADT6, At1g08250), respectively). Further confirmatory observations of a strong substrate preference for arogenate were subsequently made for one rice ADT isoenzyme (14) and three petunia ADT isoenzymes (15). Feedback inhibition of ADTs was also demonstrated to be an important factor influencing Phe biosynthesis, accumulation, and turnover as feedback insensitive ADTs in both rice and Arabidopsis were found to accumulate circa 55 and 160 times more Phe, respectively, compared to WT (14, 16).

### SUMMARY OF THE INVENTION

In particular aspects, to delineate the potential individual physiological contributions of specific arogenate dehydratase (ADT) isoenzymes, Arabidopsis lines containing knockouts (KOs) of single and multiple ADT genes were generated, with these then being analyzed for potential differential effects on phenylpropanoid metabolism (specifically lignification).

According to particular aspects, lines with a combination of ADT4 and ADT5 KOs had profoundly altered (e.g., reduced, decreased) lignin contents, including the various triple and quadruple KOs involving those isoenzymes, which gave even more pronounced (e.g., reduced, decreased) effects.

This is the first demonstration that a network pathway step (ADT) upstream of phenylpropanoid metabolism, localized in plastids/chloroplasts, can differentially alter carbon allocation/flux into lignification (phenylpropanoid metabolism) in other subcellular compartments, versus formation of Phe for either protein synthesis or some other metabolic pathway.

Provided are methods for decreasing carbon flow into lignin in plants, comprising reducing or eliminating, using mutagenesis and/or recombinant means, expression and/or activity of at least one chloroplast-localized arogenate dehydratase (ADT) sufficient to reduce phenylalanine (Phe) availability for metabolism into Phe-derived phenylpropanoids, wherein the amount, level or distribution of lignin is reduced relative to control plants. In particular aspects, the plant has a plurality of chloroplast-localized ADTs, and reducing or eliminating comprises reducing or eliminating expression and/or activity of at least two of the plurality of ADTs. Also provided are recombinant plants or a parts thereof, comprising at least one mutation, genetic alteration or transgene that reduces or eliminates the expression and/or activity of at least one chloroplast-localized ADT, wherein the amount, level or distribution of lignin is reduced relative to normal. Further provided are reduced lignin plant products.

Particular aspects provide methods for decreasing carbon flow into lignin in plants, comprising: obtaining a plant or cell thereof having at least one chloroplast-localized arogenate dehydratase (ADT) and having cytosolic and/or membrane-associated phenylpropanoid metabolism for producing phenyalanine (Phe)-derived phenylpropanoids; and reducing or eliminating, using at least one of mutagenesis and recombinant means, in the plant or the cell thereof, expression and/or activity of the at least one chloroplast-localized ADT sufficient to reduce Phe availability for metabolism into Phe-derived phenylpropanoids, wherein the amount, level or distribution of lignin in the plant or the cell thereof is reduced relative to control plants or cells thereof with normal amounts, levels or distributions of lignin, and wherein a method for decreasing carbon flow into lignin in a plant or cell thereof is afforded. In particular embodiments, the plant has a plurality of chloroplast-localized arogenate dehydratases (ADTs), and reducing or eliminating comprises reducing or eliminating expression and/or activity of at least two of the plurality of chloroplast-localized ADTs sufficient to reduce Phe availability for metabolism into Phe-derived phenylpropanoids, wherein the amount, level or distribution of lignin in the plant or the cell thereof is reduced relative to control plants or cells thereof with normal amounts, levels or distributions of lignin. In certain aspects, reducing or eliminating comprises reducing or eliminating expression and/or activity of at least three of the plurality of chloroplast-localized ADTs. In particular embodiments, reducing or eliminating comprises reducing or eliminating expression and/or activity of at least four of the plurality of chloroplast-localized ADTs, preferably wherein reducing or eliminating is by knock-out of chloroplast-localized ADTs.

In certain aspects of the methods, the at least two of the plurality of chloroplast-localized arogenate dehydratases (ADTs) correspond to phylogenetic subgroup III chloroplast-localized ADTs as defined herein. In particular embodiment, at least three of the plurality of chloroplast-localized arogenate dehydratases (ADTs) correspond to phylogenetic subgroup III chloroplast-localized ADTs as defined herein.

In certain preferred aspects, the at least one chloroplast-localized arogenate dehydratase (ADT) comprises a conserved TRF motif in the ADT active site.

According to particular embodiments, the at least one chloroplast-localized arogenate dehydratase (ADT) comprises at least one sequence selected from the group consisting of SEQ ID NOS:30, 32, 34, 36, 38, 40, 42, 44, 46, 48, 93, 50, 52, 56, 58, 60, 62, 64, 66, 68, 98, 100, 102, 104, 106, orthologs thereof, a sequence having at least 45%, at least 50%, at least 55%, at least 60%, at least 65%, at least 70%, at least 80%, or at least 85% amino acid sequence identity therewith, and ADT-active portions thereof, preferably at least 60% amino acid sequence identity therewith.

In certain aspects, the chloroplast-localized arogenate dehydratase (ADT) is that of a vascular plant. In particular embodiments, the plant or cell thereof is selected from the group consisting of hardwood, softwood, graminae and angiosperms. In certain embodiments, the plant or cell thereof is selected from the group consisting of Arabidopsis, poplar, Populus trichocarpa, pine, Pinus taeda, rice, Oryza sativa, Picea sitchensis, and Vitis vinifera.

According to certain aspects, reducing or eliminating expression of chloroplast-localized arogenate dehydratase(s) (ADT(s)) comprises use of at least one of gene-silencing, gene knock-out, anti-sense methods, siRNA methods, RNAi methods, and transgenic methods. In certain embodiments, reducing or eliminating comprises inactivating a gene normally encoding the at least one chloroplast-localized arogenate dehydratase (ADT).

According to certain embodiments, reducing or eliminating expression of chloroplast-localized arogenate dehydratase(s) (ADT(s)) comprises expression of siRNA and/or RNAi sufficient to expression and/or activity of the at least one chloroplast-localized arogenate dehydratase (ADT).

Particular embodiments comprise imparting into the germplasm of a plant variety a mutation or genetic alteration that reduces the expression or activity of the at least one chloroplast-localized arogenate dehydratase (ADT) in one or more cells of the plant, wherein the amount or level of lignin is reduced relative to control plants or cells thereof with normal amounts or levels of lignin, preferably comprising introducing into the selected variety using suitable methods a transgene that reduces the expression or activity of the at least one chloroplast-localized ADT in one or more cells of the plant relative to that of control plants or cells thereof. Certain embodiments comprise the use of T-DNA insertion.

Additional aspects provide a recombinant plant or a part or cell thereof, comprising at least one mutation, genetic alteration or transgene that reduces or eliminates the expression and/or activity of at least one chloroplast-localized arogenate dehydratase (ADT) in one or a) more cells of the plant, wherein the amount, level or distribution of lignin in the plant or the cell thereof is reduced relative to control plants or cells thereof with normal amounts, levels or distributions of lignin. In certain aspects, the plant or a part or cell thereof has a plurality of chloroplast-localized arogenate dehydratases (ADTs), and wherein the expression and/or activity of at least two of the chloroplast-localized ADTs is reduced or eliminated, preferably wherein the expression and/or activity of at least three of the chloroplast-localized ADTs is reduced or eliminated, preferably wherein the expression and/or activity of at least four of the chloroplast-localized (ADTs is reduced or eliminated.

In particular embodiments, the plant or part or cell thereof is at least one of Arabidopsis, poplar, Populus trichocarpa, pine, Pinus taeda, rice, Oryza sativa, Picea sitchensis, and Vitis vinifera. In particular embodiments, the plant or part or cell thereof is other than that of Arabidopsis.

In particular plant, plant part or cell aspects, the at least one mutation, genetic alteration or transgene that reduces or eliminates the expression and/or activity of at least one chloroplast-localized arogenate dehydratase (ADT) comprises at least one of gene-silencing, gene knock-out, anti-sense methods, siRNA methods, RNAi methods, and transgenic methods.

Further aspects provide a seed or true-breeding seed derived from the recombinant plant or part or cell thereof as disclosed and claimed herein.

Yet additional aspects provide a reduced lignin plant product derived from the plant, plant part or cell as disclosed herein, preferably wherein the plant product has less lignin relative to that of plant product derived from control plants or cells thereof with normal amounts, levels or distributions of lignin. In certain embodiments, the reduced lignin plant product comprises at least one of a fuel, food composition, antioxidant, and feedstock.

### BRIEF DESCRIPTION OF THE DRAWINGS

FIG. 1 shows RT-PCR analysis of SALK and INRA mutant lines. Gene-specific primers were used to amplify cDNA from each ADT (1-6), and an actin (A) control. Corresponding ADT mRNA was absent from adt1, adt3, adt4, adt5 and adt6; thus, each of these lines was selected for further analysis and crossing. M=molecular weight marker.

FIG. 2 shows proposed biosynthetic pathways to Phe, Tyr and Trp in plants, where the body of evidence supports the arogenate pathways to Phe/Tyr.

FIGS. 3A and 3B show growth and development parameters of Arabidopsis WT and ADT KO lines. A, stem fresh/dry weights and B, lengths measured weekly from 3.5 to 10 weeks with an average of >20 stems. Trend lines were estimated using a polynomial line of best fit. Selected examples of tissue dry weights are also provided (FIG. 2A).

FIGS. 4A through 4I show plant phenotypes at 5 weeks of growth/development. WT (A), adt1 (B), adt3 (C) and adt4 (D) lines displayed similar upright phenotypes, whereas adt5 (E), adt4/5 (F), adt1/4/5 (G), adt3/4/5 (H) and adt3/4/5/6 (I) stems were weakened, and unable to fully support their weight. Scale bars=10 cm.

FIGS. 5A through 5O show histochemical staining of stems from 7 week old WT and ADT KO lines. WT (A, G, J), adt5 (B, K), adt4/5 (C, L), adt1/4/5 (D, H, M), adt3/4/5 (E, I, N) and adt3/4/5/6 (F, O) were treated with phloroglucinol-HCl (A-I) and Mäule (J-O) regents. Positions of the interfascicular fiber (if) regions, and vascular bundles (vb) are indicated in WT (A, J). Increased magnification, of the vb in A, D and E are shown in G, H and I, respectively, with metaxylem (mx), protoxylem (px) and xylem fibers (xf) labeled in G. Fainter staining of G-moieties was detected using phloroglucinol-HCl in the if regions of adt5, adt4/5, adt1/4/5, adt3/4/5, and adt3/4/5/6 (C-F, respectively), whereas there was no apparent decrease in the vb in any KO lines. However, increased magnification (G-I) indicated that mx cell wall integrity is affected in triple KO lines (H and I), with numerous irregularly shaped/partially collapsed vessels (denoted by *) present in these lines. Scale bars: 50 μm.

FIGS. 6A through 6D show estimated lignin content/compositional analyses of Arabidopsis WT and ADT KO lines. Estimation of lignin contents using “AcBr” (A), and levels of thioacidolysis G+S- (B), G- (C) and S- (D) derived lignin monomeric cleavage products released as a function of time (growth and development).

FIGS. 7A through 7D show pyrolysis GC/MS chromatograms of WT and the adt1/4/5 KO line, showing H-, G and S-derived pyrolysis products (indicated in red, blue and green, respectively). Cell wall residues from WT (A) and adt1/4/5 (B) stem tissues, together with laser-microdissected interfascicular regions (C) and vascular bundles (D) from adt1/4/5.

FIGS. 8A through 8E show comparison of thioacidolysis-determined G- and/or S-lignin-derived monomers contents vs. total “AcBr lignin” contents, for single KO lines, adt1, adt3, adt4, and adt5, as well as multiple KO lines, adt4/5, adt1/4/5, adt3/4/5 and adt3/4/5/6. Total G+S-derived thioacidolysis monomeric degradation products compared to total “AcBr-lignin” for all single and multiple KO lines (A). G-derived thioacidolysis degradation products compared to total “AcBr-lignin” for single (B) and multiple (C) KO lines. S-derived thioacidolysis degradation products compared to total “AcBr-lignin” for single (D) and multiple (E) KO lines.

FIGS. 9A through 9E show histochemical staining of stems from 7 week old WT and ADT KO lines. WT (A, E), adt1 (B, F), adt3 (C, G), adt4 (D, H), were treated with phloroglucinol-HCl (A-D) and Mäule (E-H) regents. Positions of the interfascicular fiber (if) regions, and vascular bundles (vb) are indicated in WT (A, E). There was no apparent decrease in the staining intensity of either the vb or the if in the ADT KO lines relative to WT. Scale bars: 50 μm.

FIGS. 10A through 10F show relative expression levels of Arabidopsis ADTs (1-6) in 5 week old stems of WT and ADT KOs. Expression levels of ADT1 (A), ADT2 (B), ADT3 (C), ADT4 (D), ADT5 (E), ADT6 (F) are shown for each line, as indicated: WT, adt1, adt3, adt4, adt5, adt4/5, adt1/4/5, adt3/4/5, and adt3/4/5/6. Each is shown relative to the ADT1 expression level in WT, with each value representing a mean±SE of 3 replicates of stems pooled from 4-6 plants. Two-tailed Student's t-tests were performed, with closed or open circles representing significant increases or decreases in expression levels, respectively, relative to WT (P<0.05).

FIG. 11 shows pyrolysis GC/MS lignin-derived products 1-29.

FIG. 12 shows the pK7GWIWG2(II) Gateway vector used in EXAMPLE 15 for RNAi expression in poplar.

FIG. 13 shows forty- to fifty-day-old, in vitro grown hybrid poplar (Populus tremula×P. alba, INRA 717-1B4, female).

FIGS. 14A and 14B show (A) callus induction on CIM2 medium for 21 days, and (B) shoot induction on SIM medium for 21 days.

FIGS. 15A and 15B show (A) adventitious shoot clumps with visible leaflets on SIM medium after 42 days, and (B) shoot elongation on SEM medium for 21 days.

FIGS. 16A and 16B show (A) root induction on RIM medium, and (B) thirty days grown transgenic hybrid poplar.

FIG. 17 shows a transgenic hybrid poplar in a growth chamber.

FIG. 18 shows an alignment of ADTs from Arabidopsis, Pine, Poplar, Rice, showing the ADT domain in green; ACT domain in red; and sequences conserved in >50% of samples are shaded in blue, where darker shades represent greater conservation. Sequences, from top to bottom in each panel are as follows: SEQ ID NO:70; SEQ ID NO:86; SEQ ID NO:75; SEQ ID NO:76; SEQ ID NO:77; SEQ ID NO:78; SEQ ID NO:69; SEQ ID NO:87; SEQ ID NO:82; SEQ ID NO:83; SEQ ID NO:72; SEQ ID NO:73; SEQ ID NO:71; SEQ ID NO:74; SEQ ID NO:77; SEQ ID NO:78; SEQ ID NO:79; SEQ ID NO:89; SEQ ID NO:88; and SEQ ID NO:90.

FIG. 19 shows phylogenetic clustering of ADTs.

FIG. 20 also shows phylogenetic clustering of ADTs.

### DETAILED DESCRIPTION OF EXEMPLARY EMBODIMENTS

How carbon flux differentially occurs in vascular plants for protein synthesis, phenylpropanoid metabolism (i.e. lignins) and other metabolic processes is not well understood. Applicants previously discovered/validated that a six-membered arogenate dehydratase (ADT 1-6) family encodes the final step in Phe biosynthesis in Arabidopsis thaliana, and this raised the hypothesis as to whether individual ADT isoenzymes (or combinations thereof) differentially modulated carbon flux to lignins, proteins, etc. If so, unlike all other lignin pathway manipulations which target cell wall/cytosolic processes, this would be the first example of a plastid (chloroplast) associated metabolic process influencing cell-wall formation. Homozygous T-DNA insertion lines were thus obtained for 5 of the 6 ADTs, these being used to generate double, triple and quadruple knockouts (KOs) in different combinations. The various mutants so obtained gave phenotypes with profound but distinct reductions in lignin amounts, encompassing a range spanning from near to wild type levels to reductions of up to 68%. In the various KOs, there were also marked changes in guaiacyl (G):syringyl (S) ratios ranging from ˜4:1 to 1:1, respectively, these being rationalized due to differential carbon flux into vascular bundles (vb) versus that of fiber cells. Laser-microscope dissection/pyrolysis GC/MS, coupled with histochemical staining/lignin, analyses, also suggested that ADT5 mainly affects carbon flux into the vb, whereas the adt3456 knockout additionally greatly reduced carbon flux into fiber cells. This plastid-localized metabolic step thus profoundly differentially affects carbon flux into the lignins in distinct anatomical regions, and provides incisive new insight into different factors also affecting G:S ratios.

### Exemplary ADT Manipulations and Phenotypic Effects

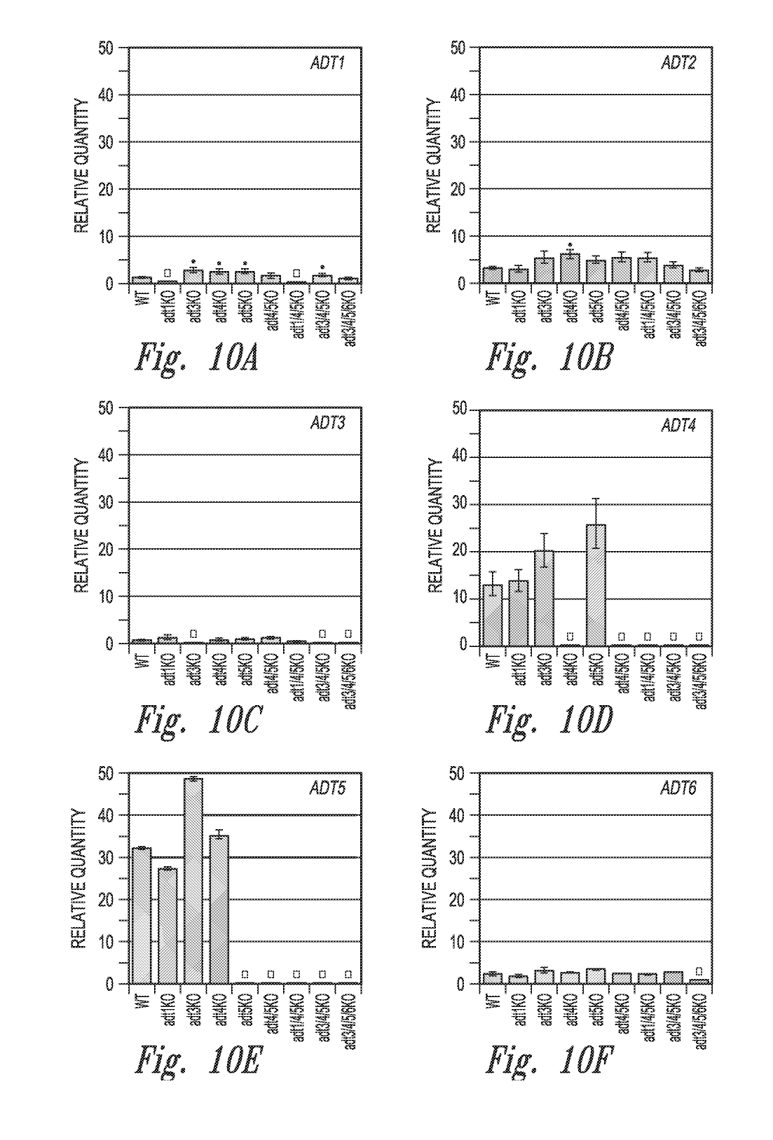

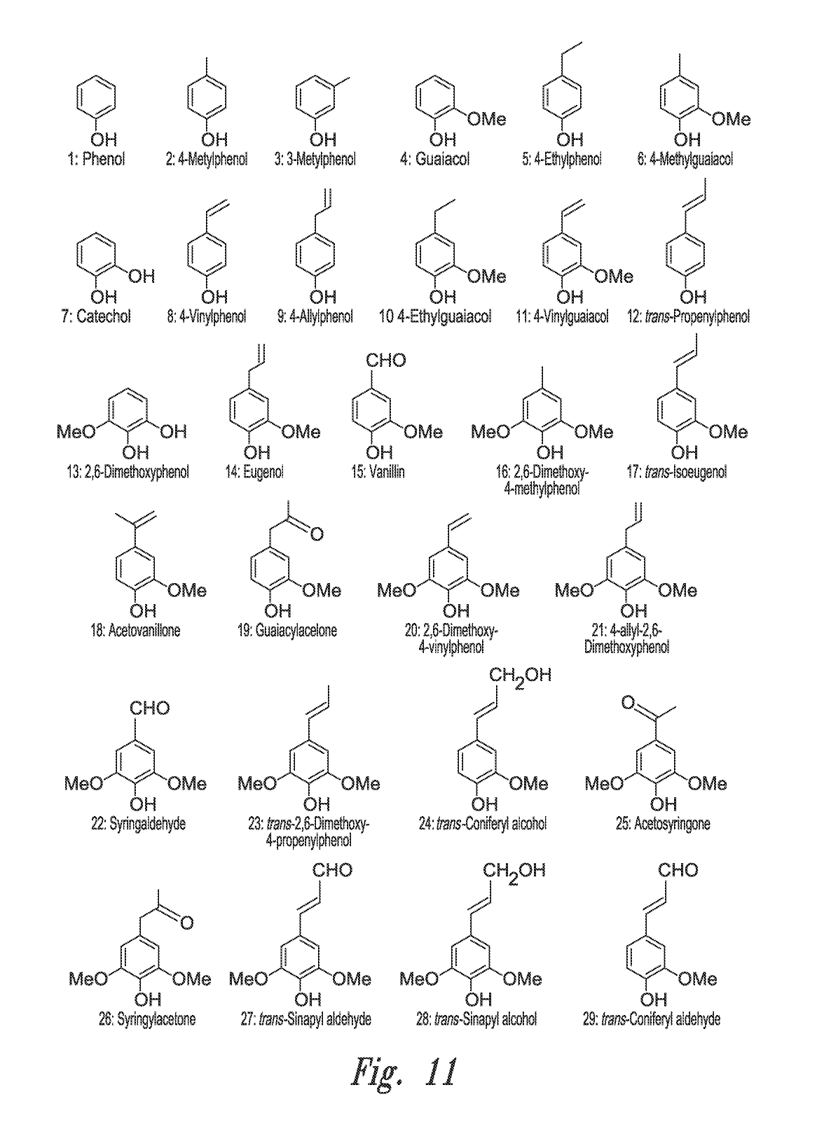

The possible differential contribution of distinct ADTs in impacting upon carbon flux into the lignin pathway was investigated given its branch point position between shikimate and phenylpropanoid metabolism. According to particular aspects, it was confirmed by real-time RT-PCR that ADT mRNA transcripts were below or near below detection from each corresponding ADT KO line examined (adt1, adt3, adt4, adt5, adt4/5, adt1/4/5, adt3/4/5 and adt3/4/5/6) (FIGS. 10A-10F) (see, e.g., EXAMPLES 1, 3 and 8 below for a detailed discussion of generation of the KO lines and real-time expression analysis). While slight increases in non-targeted ADTs were observed in certain ADT KO lines, there was no apparent trend for increased ‘compensation’ in the double, triple and quadruple KOs, relative to the single KOs. This was clearly demonstrated in the adt3/4/5/6 KO line, for which ADT3, ADT4, ADT5 and ADT6 transcript levels were decreased/abolished, but the remaining ADT1 and ADT2 transcript levels were identical to those of WT. These results indicate that the observed phenotypes are a direct result of diminished ADT gene expression levels in the knocked out ADTs. The decrease in ADT expression is fully consistent with the phenotypes observed for these plants.

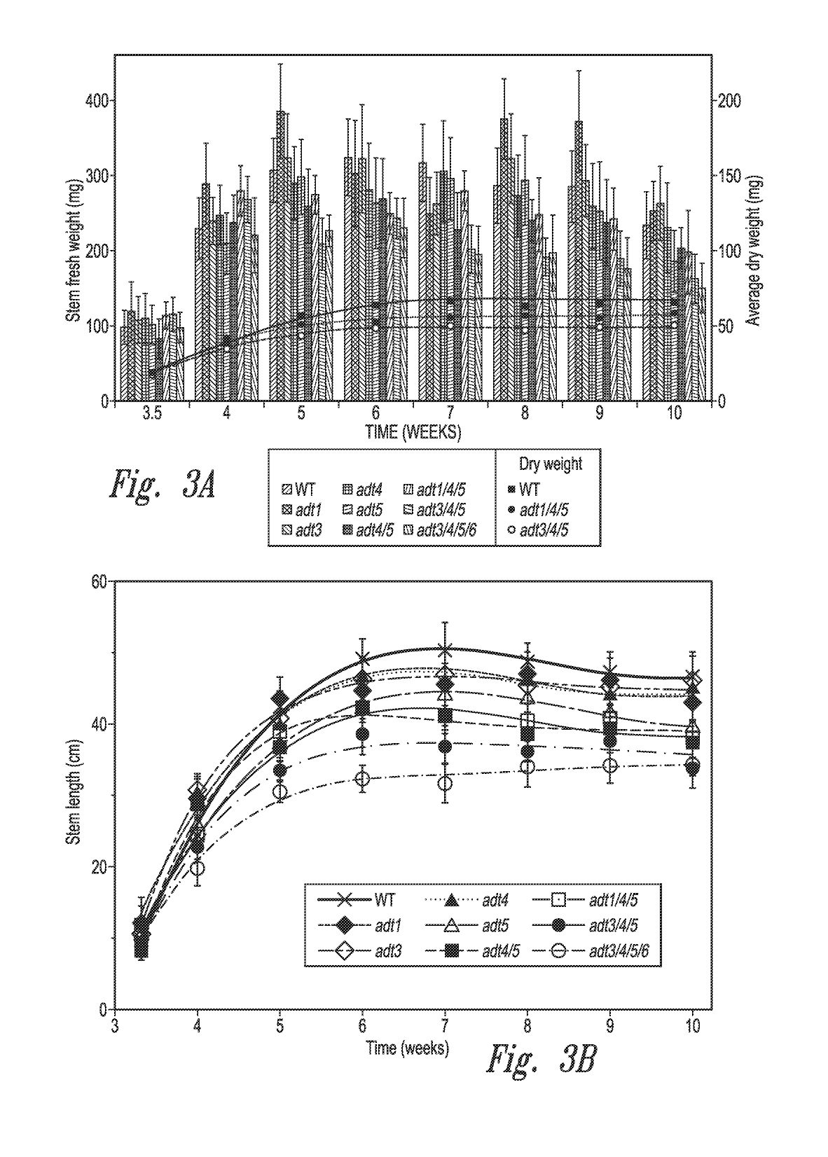

Depending upon the plant line generated, there were relatively significant, but distinct, reductions in stem lengths (to a range of circa 70-83% of WT, see FIG. 3B; EXAMPLE 8), when adt5 was ‘knocked out’ in combination with adt4, adt1/4, adt3/4, and adt3/4/6, respectively. This was also noted in dry weight stem tissue determinations which showed reductions of circa 14 and 24% for adt1/4/5 and adt3/4/5, relative to WT. Such relatively small effects on biomass production, however, contrast with numerous other studies on monolignol pathway step modulations. The latter frequently result in, for example, extremely dwarfed plant lines (with greatly reduced biomass) and significantly compromised vasculature (for a discussion see reference 7).

### Comparison of Histochemical and Pyrolysis GC/MS Analyses of Vascular Bundles and Interfascicular Fibers

Guaiacyl and S entities are often qualitatively detected using either the histochemical staining reagent, phloroglucinol-HCl, for G-components in lignified tissues, or the Mäule reagent for S-derived lignin moieties. Histochemical staining of the various lines generated herein (adt1, adt3, adt4, adt5, adt4/5, adt1/4/5, adt3/4/5 and adt3/4/5/6) on comparison to WT, thus provided some useful insights into the limitations of these staining protocols (see EXAMPLES 5 and 10 below). Specifically, the double, triple and quadruple mutants containing the adt5 knockout gave essentially no indication that G-moieties were present in if regions, whereas the vb all stained positively even though there was considerable distortion/weakening of the metaxylem (mx) cell walls (FIGS. 5B-5F). By contrast, for each of the lines examined using the Mäule reagent, S-staining was notably detected in if cell walls, as well as in fiber-containing cell walls within the vb (FIGS. 5K-5O). These data thus suggested the absence of G-moieties in the if regions.

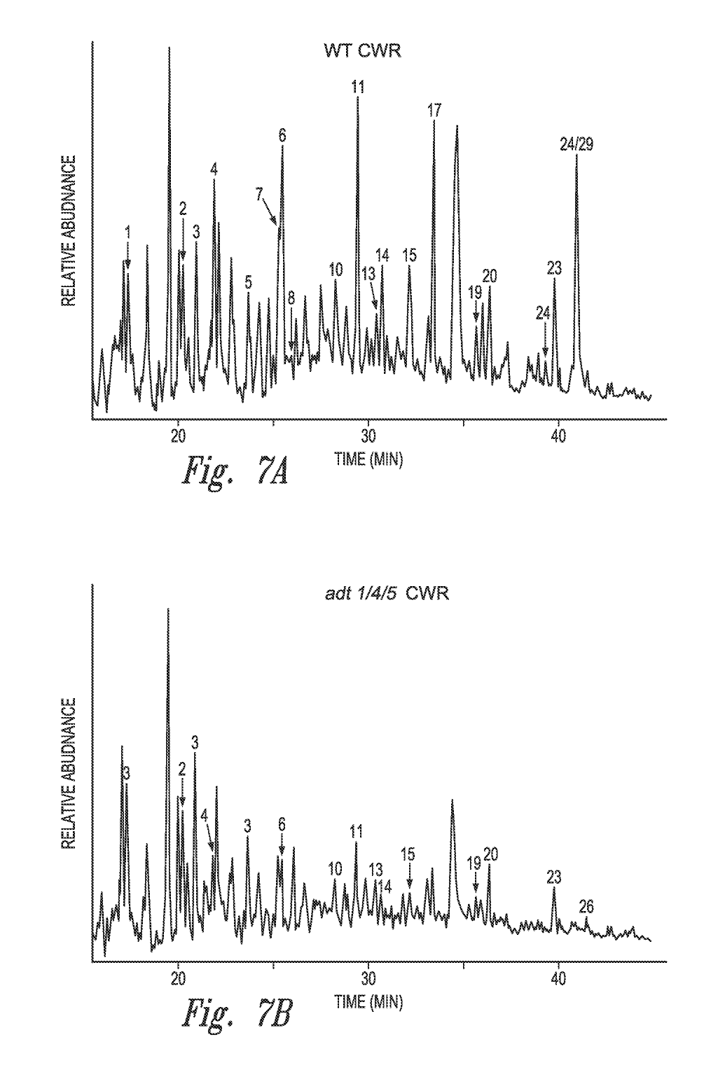

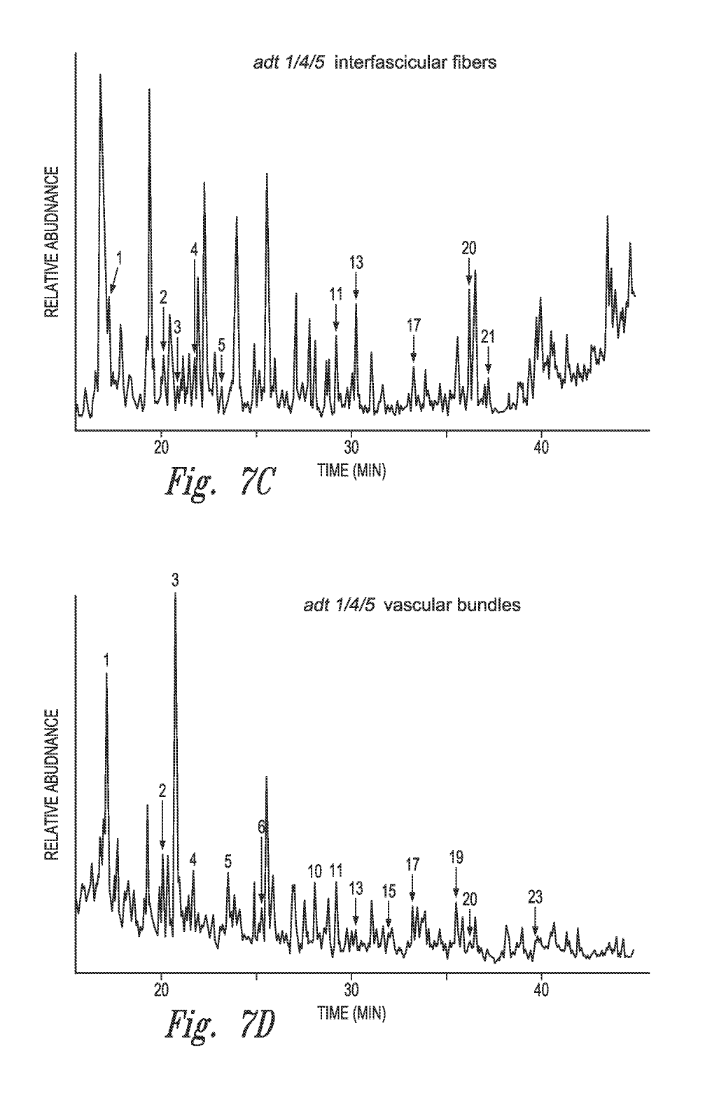

Pyrolysis GC/MS of stem cross-sections (CWRs) of both WT and adt1/4/5 were also carried out, and these resulted in facile detection of various H, G, and S-derived monomers (FIGS. 7A and 7B; Table 1) (see EXAMPLES 7 and 14 below). By comparison to the H-derived constituents, however, the levels of both G- and S-derived moieties were much reduced overall in the adt1/4/5 line, with the highest reduction being in G-derived moieties (FIG. 7B). Laser-microscope dissected if and vb sections of both WT and adt1/4/5 lines, when subjected to pyrolysis GC/MS, next provided considerable insight into the type of lignins present in these distinct anatomical regions. For the if region of adt1/4/5, it was evident (relative to H-derived monomers) that the amounts of G-derived pyrolysis products were substantially diminished relative to S-derived moieties, with the latter being comparatively more readily detectable (FIG. 7C). Thus, the if regions still contained a S-enriched lignin even though overall lignin amounts were substantially reduced. Failure to detect G-moieties in if regions of some of these mutants by histochemical staining thus demonstrates a serious limitation in this qualitative staining protocol, when G-levels are low (relative to S- and H-derived moieties).

Laser microscope dissection/pyrolysis GC/MS of WT if regions resulted in facile detection of H- (peaks 1 and 3), G- (peaks 4, 6, 7, 10, 11, 14, 15, 17-19 and 24) and S- (13, 20-23, 27 and 28) lignin derived moieties, with the G- and S-moieties predominating relative to the H-derived component (data not shown, Patten et al., 2010). By contrast, analysis of the if regions of the adt1/4/5 indicated substantial reductions in G-component release, with both S and H component detection being relatively comparable. Pyrolysis GC/MS of WT vb regions indicated the presence of presumed H— (peaks 1 and 3), G- (peaks 4, 6, 11, 14, 15, 17-19, and 24) and S-(peaks 13, 20-23 and 27) lignin-derived moieties (data not shown, Patten et al., 2010). In the adt1/4/5 vb (FIG. 7D) it was evident that, compared to the H-pyrolysis products, both G and S moieties were also significantly reduced in amounts with the S-components being barely detectable. However, the G moieties still predominated. The pyrolysis data are thus in general agreement with the bulk studies of the “AcBr lignin contents” and monomeric release of G- and S-derived (8-O-4′ interunit cleaved) thioacidolysis products in the different lines.

### ADT Manipulations and Effect on Lignification

Total lignin monomeric compositions and contents were also systematically studied over a period of 3-10 weeks, reflecting the three phases of Arabidopsis growth/development until maturation and senescence. As indicated above, massive, yet differential, reductions in lignin contents and compositions were observed, via manipulation of this plastid-localized enzyme family (see, e.g., EXAMPLES 7 and 11-13 below).

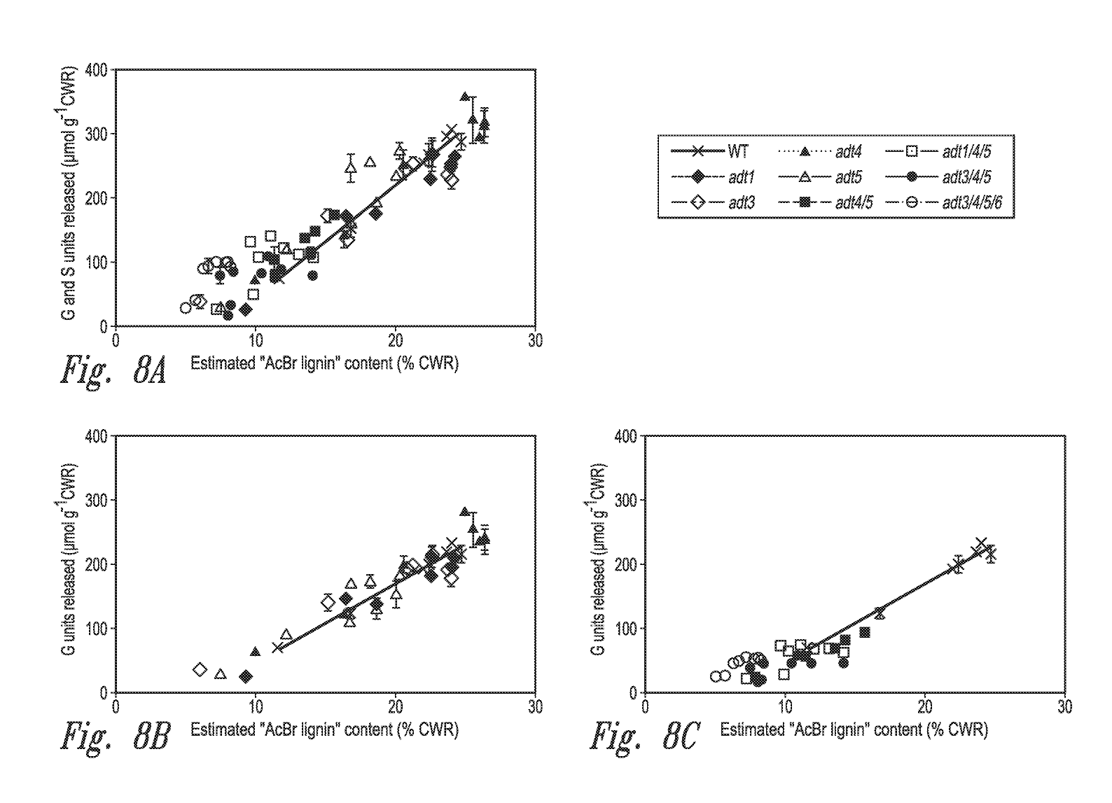

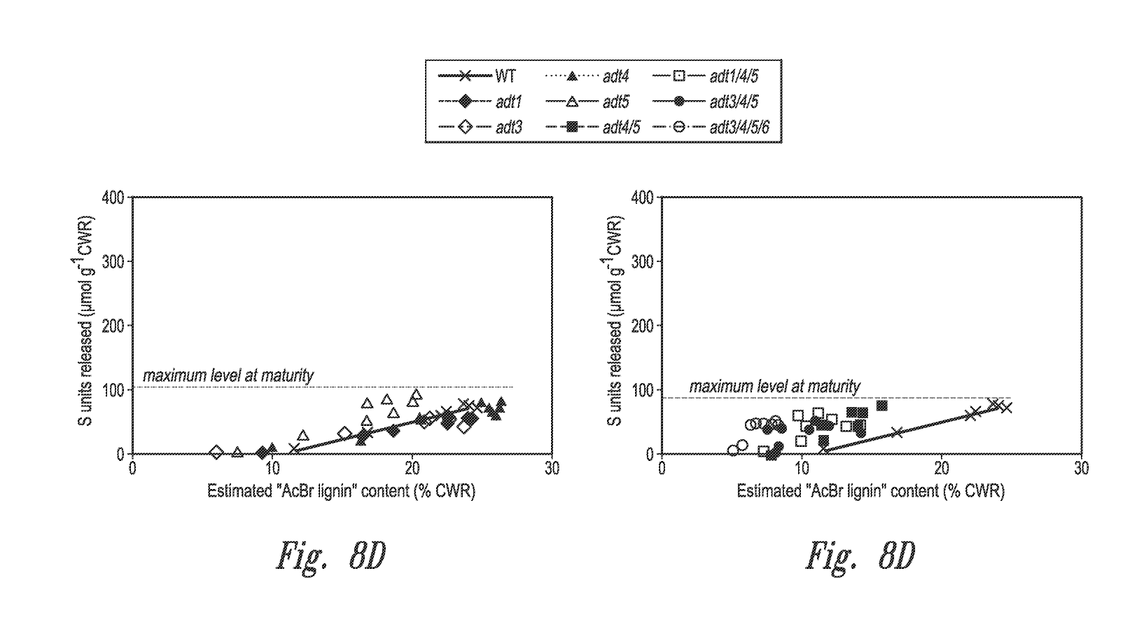

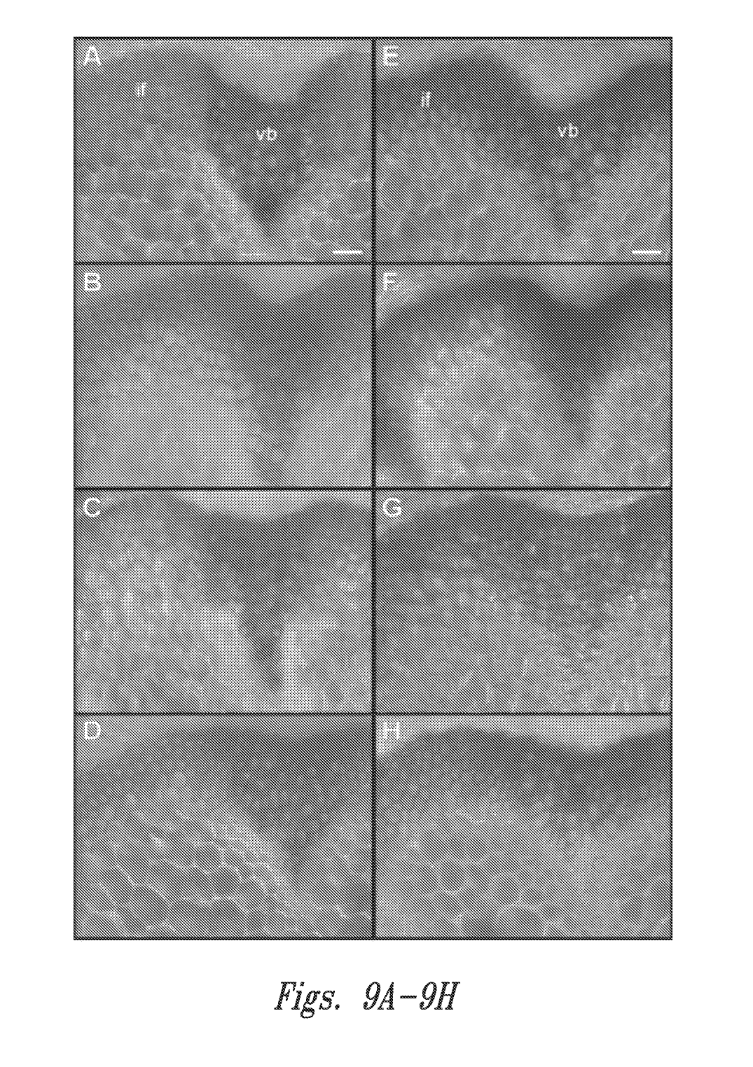

Applicants next plotted out correlations between estimated “AcBr lignin” contents versus thioacidolysis released G+S monomer levels in the different lines generated at different growth stages (sampled weekly) until the plant stems matured and ultimately senesced (FIG. 8A) (see, e.g., EXAMPLES 11 and 12 below). Although there was considerable experimental variation in samples tested, all of the lines examined essentially gave linear increases overall in releasable G+S monomer amounts relative to estimated lignin contents, as previously observed (7, 17). At maturation, however, the adt4/5 (▴), adt1/4/5 ( [symbol] ), adt3/4/5 () and adt3/4/5/6 ( [symbol] ) lines had G+S monomer release/“AcBr lignin” levels that did not surpass the 4 week old (Stage 2) levels for the WT line. As documented elsewhere (17), there was also an initial ˜5% “AcBr lignin” deposition, where essentially no G+S moieties are released; this early stage “AcBr lignin”, presumed H-derived (from p-coumaryl alcohol), can also include other non-lignin components, as discussed in Jourdes et al. (18) and Davin et al. (7). Most importantly though, are the essentially linear correlations of G+S monomer release versus estimated lignin contents are considered indicative of a biochemical process in place leading to (near) conservation of the 8-O-4′ inter-unit linkage frequency in these various lignins. This again reflects control exercised over lignin macromolecular assembly (7).

It was instructive to also compare G and S monomer release from the different lines generated. As discussed earlier above (Results), the adt5 KO (♦) had the largest (of any single adt mutant) reductions in lignin contents and thioacidolytic monomer release (FIGS. 8B and 8D), relative to WT (see, e.g., EXAMPLE 13 below). However, the effect on reductions in monomer release essentially only impacted G-monomer levels (circa 74% of WT, FIG. 8B) but not that of the S-constituents, which (if anything) were slightly increased (FIG. 8D). These data thus provisionally suggested that, at a minimum, essentially the same amount of cleavable S-lignin-derived monomers was being generated in the adt5 line as for WT. The overall amounts of G and S moieties released from adt1 ( [symbol] ) adt3 ( [symbol] ) and adt4 ( [symbol] ) lines were also similar to that of WT at maturity, albeit with a slight increase in levels of G-moieties for adt4 (FIGS. 8B and 8D). In the latter case, there also appeared to be a small increase in total ‘AcBr’ lignin contents (circa 10% more relative to WT). Given the large G-monomer reduction levels in adt5, but not on S-levels, these data provisionally suggested that the primary effect was on the lignin forming biochemical machinery in the G-enriched vb rather than in the various regions containing fiber cells.

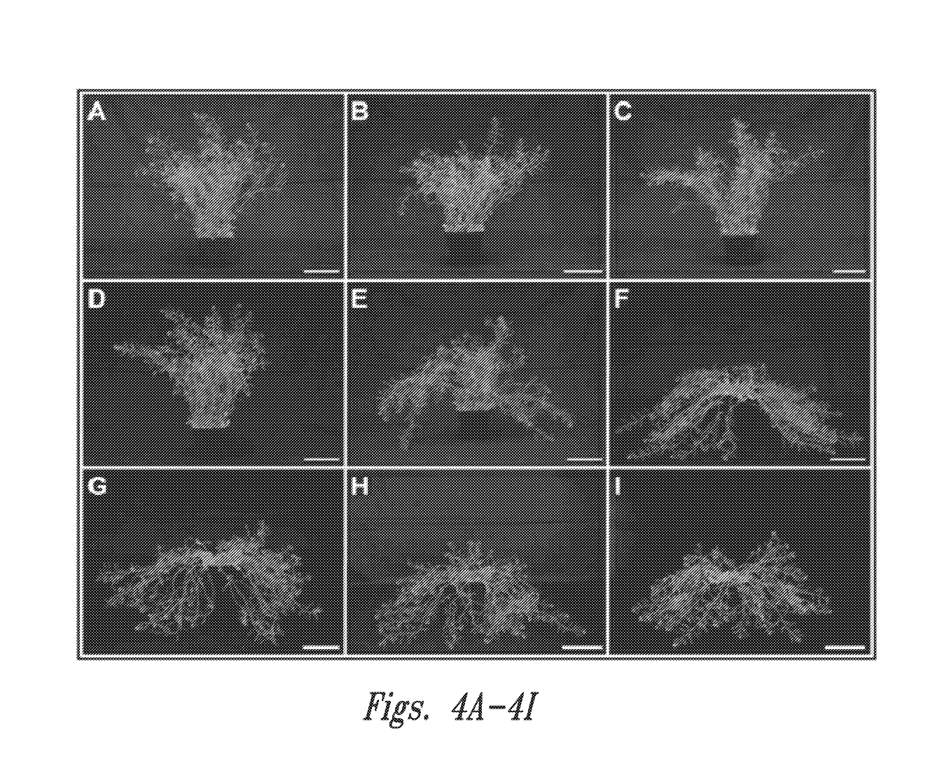

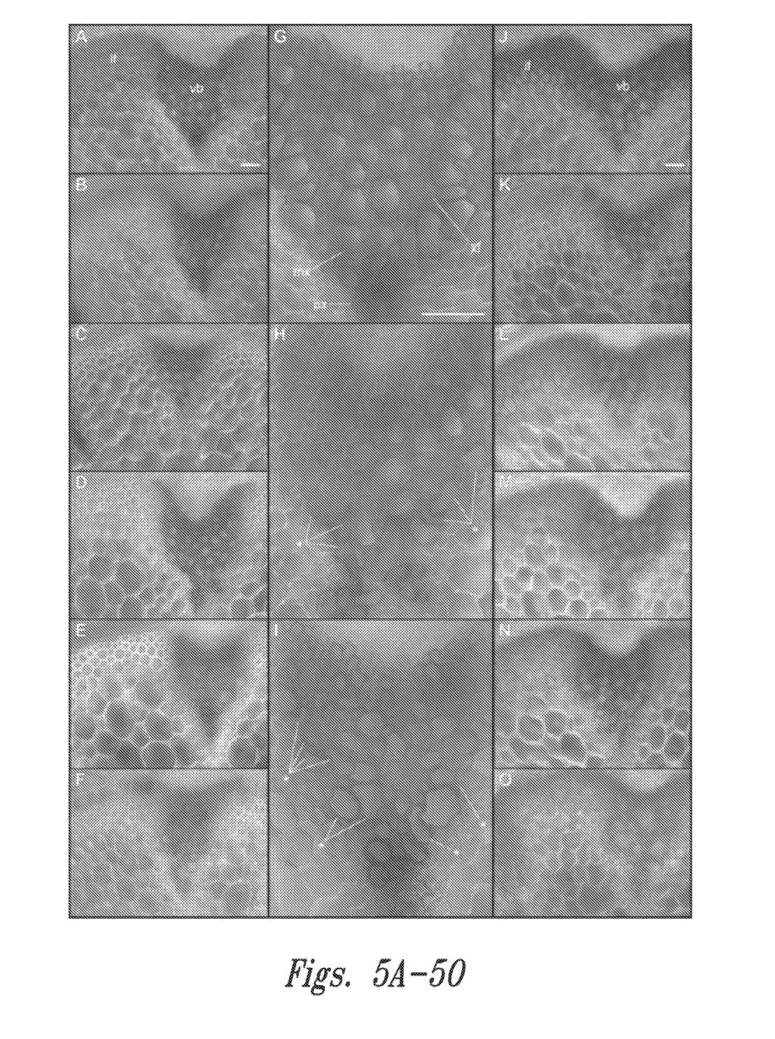

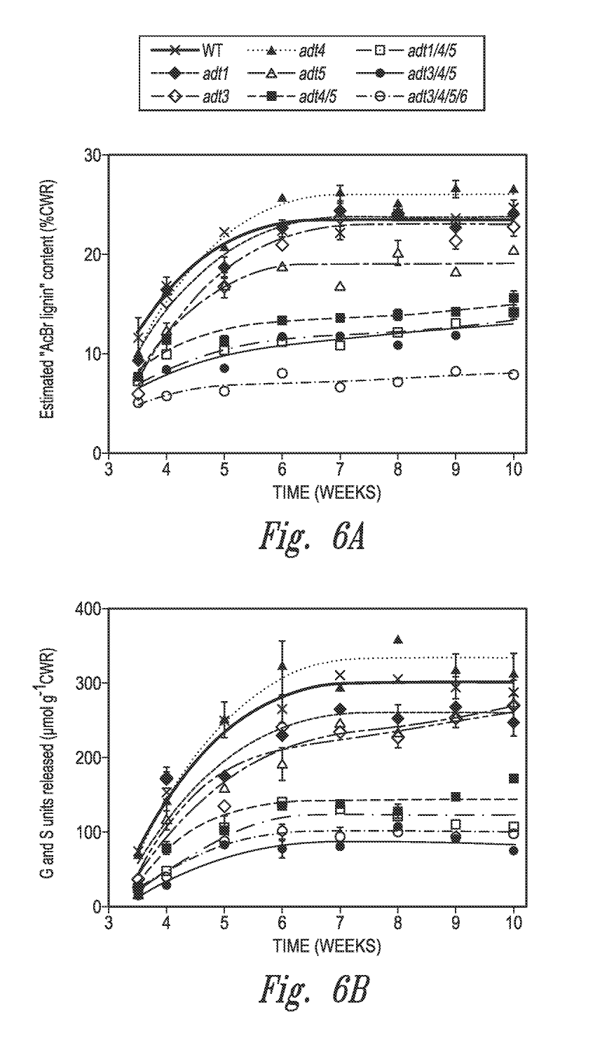

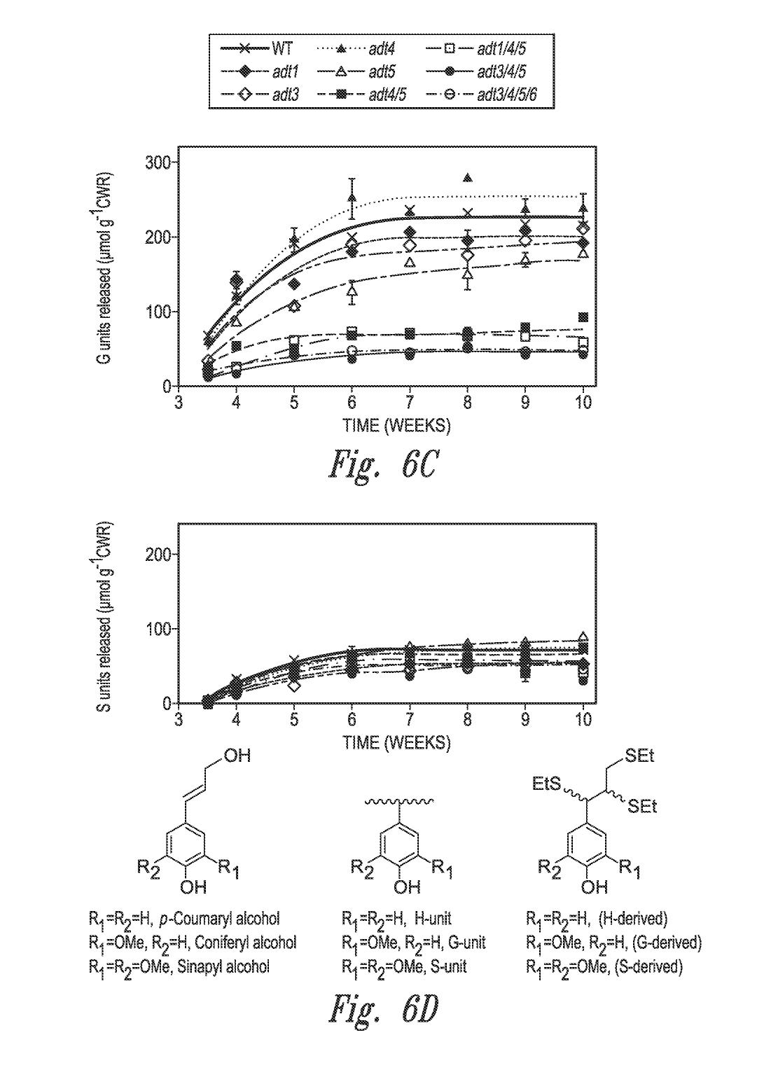

Examination of the double, triple and quadruple KOs, all of which contained the adt5 KO, was very also informative, as this added and extended to the observations made above (FIGS. 8C and 8E). As indicated earlier, the first of these, adt4/5 (A) displayed a quite pronounced prostrate phenotype (FIG. 4D), and also at maturity had ‘AcBr’ lignin contents estimated to be ˜61% of WT (FIG. 6A). However, at maturity, there was again essentially no difference in the amount of releasable S-monomers, relative to WT, whereas by contrast the G-monomers released were reduced to circa 35% of WT levels (FIGS. 8C and 8E). These data were thus again consistent with the primary target for lignin and G-monomer reduction being within the vb region.

For the triple (adt1/4/5 and adt3/4/5) and quadruple (adt3/4/5/6) knockouts, the reductions in releasable G-monomer levels were reduced further down to circa 29, 20, and 21% relative to WT at maturity, whereas the S-releasable monomers were now also reduced to circa 72, 58 and 67% of WT amounts, respectively. Thus, with the triple and quadruple knockouts there was now clearly also an effect on S-monomer amounts and hence on (S) lignin deposition in the fiber cells. Overall, the lignin contents in these lines were also significantly reduced down to ˜52, 50 and 32% of WT levels, respectively, this representing some of the largest reductions in lignin levels ever reported in genetic manipulations.

Taken together, these data again point to different modulation of ADT-regulated carbon flux into distinct anatomical regions (vb vs. fiber cells). As a result, it was not unexpected that the G:S ratios in the lignins so obtained also changed markedly with different KO lines from ˜3:1 (WT) to ˜1:1 (adt3/4/5/6). This was because these cell types still contained the biochemical machinery to predominantly generate G- and S-derived monomers, respectively, i.e. thereby generating different amounts of G- and S-lignins.

Such differential effects on lignin deposition are striking, however, given the different subcellular localizations of ADTs in chloroplasts/plastids for Phe formation versus monolignol pathway enzymes being cytosol/membrane localized. This is the first time though that a plastid/chloroplast localized upstream step involving the shikimate-chorismate pathway has been shown to have specific isoenzymes dedicated to profoundly and differentially directing carbon flux into Phe for lignin biosynthesis (19, 20). There was again no evidence for “compensatory combinatorial chemistry” in lignin biosynthesis as has often been repeatedly claimed (21), as carbon allocation to the pathway was simply significantly reduced with the resulting phenotypes substantially weakened from a structural vasculature viewpoint due to their decreased lignin levels.

### Identifying Physiological Roles of ADT Isoenzymes and ADT Phylogeny

After translation, the various ADT isoenzymes in Arabidopsis are targeted to chloroplasts/plastids (13). The ADTs are postulated to form tetrameric structures, based on structural comparison to Staphylococcus aureus PDT (22, 23), that are feedback sensitive to Phe (2, 3). Whether these are homotetramers or heterotetramers is, however, unknown. Applicants' earlier phylogenetic tree comparisons (1) also indicated that ADT3 and ADT6 are in the same cluster (subgroup III) as ADT4 and ADT5, whereas ADT1 and ADT2 are in subgroups 1 and 2, respectively. Yet while knocking out all four genes in subgroup III resulted in the greatest reductions in lignin levels, the adt3/4/5/6 quadruple KO did not significantly reduce further the remaining estimated ˜29% lignin (present in the adt3/4/5 KO) as gauged by G+S monomer release. That is, there was no complete depletion of releasable G- and S-monomers in the various knockout lines examined, this, in turn, raising interesting questions about the role of the remaining isoenzymes, including ADT2. While Applicants currently envisage a primary role for it in protein formation, it is also possible that the H-components in lignin are largely derived from ADT2 generated Phe, as well as being responsible for some of the carbon in the “residual” G+S lignins being formed.

To date, all six Arabidopsis ADT genes were found to be expressed in stems, leaves, roots, flowers, siliques and seeds (1, 13). Of these, ADT2 was the most highly expressed, and was also suggested earlier to have a “housekeeping” role in Phe biosynthesis (13). By contrast, ADT4 and ADT5 were more highly expressed in stems and roots, whereas the remaining isoenzymes had generally much lower levels of expression (13).

### Participation of ADT Isoenzymes in Distinct Metabolic Networks

Co-expression analyses are becoming increasingly common in searches for putative gene networks, although these frequently do not provide unambiguous proof of network relationships without additional experimental approaches being pursued. Nevertheless, while the physiological significance was unknown, the arbitrarily annotated ADT3, ADT5 and ADT6 were identified using global transcript profiling as being upregulated during stem growth (24), and ADT4 and ADT5 were apparently co-regulated with phenylpropanoid genes C4H (25) and COMT1 (26).

In order to begin to identify further potential metabolic relationships for the ADT genes, the Botany Array Resource Expression Angler (27) was also employed herein, using each of the six ADTs as bait (Table 5).

TABLE 5 shows potential co-expression relationships using Expression Angler.^{a} ADT1-6 were used as ‘bait’ to identify potentially co-regulated genes. Genes of interest with potential involvement in the shikimate-phenylpropanoid pathway are shown in Table 5A, while primary metabolism and cell-cycle related genes are shown in Table 5B. For each table, the range of r-values for the 100-most closely co-regulated genes is listed in the left hand column; each gene also has an r-value listed in brackets. These values give a relative value for how closely the two genes are co-regulated. Note that ADT1 and ADT2 have much higher r-values; however, this is due to their relatively low, constitutive expression patterns, which tends to yield more matches with higher r-values (27).

This provided further supporting evidence for the differential involvement of ADT genes in distinct metabolic networks. In these analyses, the most highly co-regulated genes segregated into two groups; those with a known or putative role in the shikimate-phenylpropanoid pathway (Table 5A), and those with a known or putative role in primary metabolic/cell cycle processes (Table 5B).

Specifically, genes encoding ADT3, ADT4, ADT5 and ADT6 were provisionally co-regulated with numerous shikimate, phenylpropanoid, and aromatic amino acid biosynthesis genes (Table 5A). ADT3 and 6 were also tightly co-regulated with each other, and both were co-regulated with several flavonoid biosynthetic genes, suggesting they may also participate in those branches of the phenylpropanoid pathway (Table 5A). There were also several isoprene/terpenoid biosynthetic genes apparently co-expressed with ADT3 and 6, which have a distinct biosynthetic pathway from phenylpropanoids. It is not clear at this time though if there is a functional relationship behind their provisional co-expression, or if it is simply coincidental. Additionally, while ADT4 and 5 were not closely co-expressed, each was provisionally co-expressed with at least one gene of each step in the monolignol pathway between Phe and the three monolignols 1-3, with the exception of F5H (Table 5A). Thus, this analysis further indicates (at the level of transcription) a role for subgroup III ADTs in Phe biosynthesis to primarily meet the demands of lignin deposition and perhaps other forms of secondary metabolism as well.

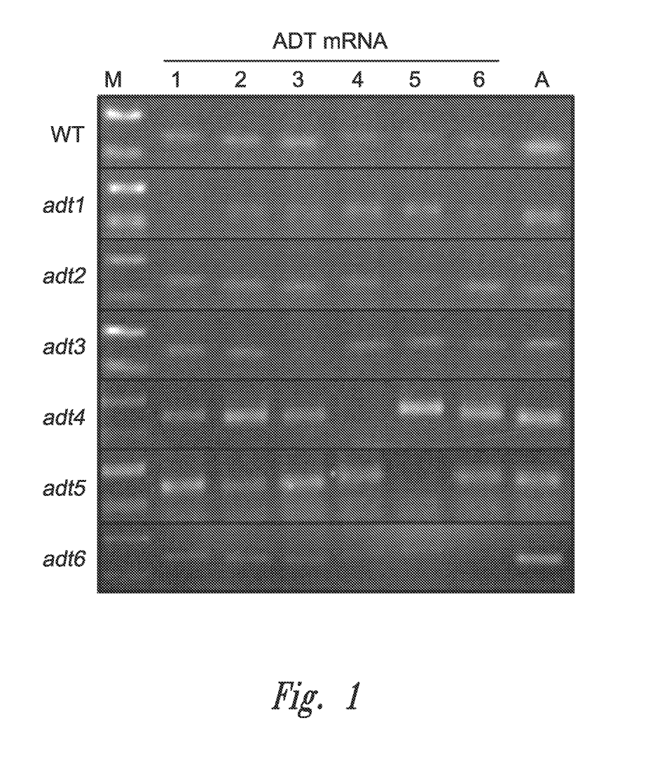

ADT3, 4, 5 and 6 were, however, not closely co-expressed with primary metabolic genes, other than those involved in aromatic amino acid biosynthesis, suggesting that they may not participate in the production of Phe for basic cellular processes (i.e. protein biosynthesis) (Table 5B). By contrast the most similarly-expressed genes with both ADT1 and 2 mainly involved basic cellular functions, such as transcription, translation, cell division, and nucleic or amino acid biosynthesis (Table 5B). Neither were, however, co-regulated with any genes involved either in the phenylpropanoid or flavonoid pathways (Table 5A). These results suggest that ADT1 and 2 are probably instead involved in primary metabolic functions in the cell (i.e. supplying Phe for protein synthesis). Indeed, in particular, this may explain why no T-DNA line was available from SALK for ADT2, and why the resulting homozygous line of the T-DNA insert in the putative promoter region of ADT2 from INRA did not successfully knockout the ADT2 transcript (FIG. 1). Taken together, these results provisionally support “housekeeping” roles for subgroup I and II ADTs mainly directed to Phe biosynthesis for primary metabolism.

TABLE 5A
Exemplary genes of interest potentially co-regulated with ADTs involved in
phenylpropanoid production (r-values)

| | Shikimate/Aromatic | | | Known/Putative | |
|---|---|---|---|---|---|
| | Amino Acid | Phenylpropanoid | Flavonoid | Phenylpropanoid | AA/NH₃/NO₃ |
| Bait | Pathway | Pathway | Pathway | TFs | Transport |
| ADT1 | | | | ATMYC1, | Amino acid |
| Top 100 | | | | At4g00480 | permease family |
| r = .959 | | | | (.867) | protein, |
| to .844 | | | | | At1g31830 (.855) |
| ADT2 | | | | | Trp/Tyr permease |
| Top 100 | | | | | family protein, |
| r = .946 | | | | | At5g19500 (.808) |
| to .641 | | | | | |
| ADT3 | Arogenate | UDP-glucosyl | Flavone 3- | Basic helix-loop- | Ammonium ion |
| Top 100 | dehydratase (ADT) | transferase (UGT) | hydroxylase | helix (bHLH) | transporter (AMT) |
| r = .570 | 6, At1g08250 | 84A2: sinapate | (F3H)^{c}, | family protein | 1;2, At1g64780 |
| to .416 | (.567); | glucosyltransferase^{b}, | At3g51240 | At4g17880 | (.488); |
| | Arogenate | At3g21560 (.495); | (.467); | (.468); | Nitrate transporter |
| | dehydrogenase | Cinnamyl alcohol | UGT78D2^{d,e} | Late elongated | (NTP) 3, |
| | (ADH), At1g15710 | dehydrogenase | At5g17050 | hypercotyl | At3g21670 (.454) |
| | (.423) | (CAD) 4, | (.454); | (LHY) 1, | |
| | | At3g19450 (.481); | Chalcone- | At1g01060 | |
| | | 4-Coumarate CoA | flavone | (.445) | |
| | | ligase (4CL) 3, | isomerase | | |
| | | At1g65060 (.459) | (CHI) family | | |
| | | | protein^{f}, | | |
| | | | At5g05270 | | |
| | | | (.444); | | |
| ADT4 | Indole-3-glycerol | Cinnamoyl-CoA | Phytoalexin | WRKY75, | AMT 2;1, |
| Top 100 | phosphate synthase | reductase (CCR) 2, | deficient | At5g13080 | At2g38290 (.584); |
| r = .811 | (IGPS), At2g04400 | At1g80820 (.667); | (PAD) 3, | (.763); | NTP2.6, At3g45060 |
| to .641 | (.811); | 4CL5, At3g21230 | At3g26830 | WRKY15, | (.584) |
| | Anthranilate synthase | (.666); | (.753) | At2g23320 | |
| | (AS) αS1, | CAD5, At4g37990 | | (.700); | |
| | At5g05730 (.767); | (.633) | | bHLH family | |
| | Trp synthase αS1, | | | protein, | |
| | At3g54640 (.697); | | | At1g10585 | |
| | Tyr aminotransferase | | | (.668); | |
| | (TAT) 3, At2g24850 | | | WRKY46, | |
| | (.661); | | | At2g46400 | |
| | 3-Deoxy-D-arabino- | | | (.658); | |
| | heptulosonate | | | WRKY6, | |
| | (DHQ) Synthase, | | | At1g62300 | |
| | At4g39980 (.652); | | | (.647); | |
| | AS βS-like, | | | Arabidopsis NAC | |
| | At1g24807 (.624); | | | domain | |
| | DAHP synthetase- | | | containing | |
| | like, At1g22410 | | | protein (ANAC) | |
| | (.618) | | | 19, At1g52890 | |
| | | | | (.641) | |
| ADT5 | DAHP synthetase- | 4CL2 (.707); | PAD3, | bHLH family | Acidic amino acid |
| Top 100 | like, At1g22410 | Phe ammonia lyase | At3g26830 | protein, | transmembrane |
| r = .714 | (.651); | (PAL) 1, At2g37040 | (.543); | At5g57150 | transporter, |
| to .548 | ADT4, At3g44720 | (.623); | | (.627); | At5g63850 (.543) |
| | (.551); | Hydroxycinnamoyl- | | MYB7, At2g16720 | |
| | AS αS1, At5g05730 | CoA | | (.609); | |
| | (.545) | shikimate/quinate | | MYB122, | |
| | | hydroxycinnamoyl | | At1g74080 | |
| | | transferase (HCT), | | (.607); | |
| | | At5g48930 (.622); | | WRKY39, | |
| | | Caffeoyl CoA 3-O- | | At3g04670 | |
| | | methyl-transferase | | (.579); | |
| | | (CCoAOMT) 1, | | ERF1, At4g17500 | |
| | | At4g34050 (.615); | | (.577); | |
| | | p-Coumarate 3- | | MYB50, | |
| | | hydroxylase (C3H), | | At1g57560 | |
| | | At2g40890 (.597); | | (.563); | |
| | | 4CL5, At3g21230 | | ANAC072, | |
| | | (.562); | | At4g27410 | |
| | | Cinnamate 4- | | (.549); | |
| | | hydroxylase (C4H), | | ANAC053, | |
| | | At2g30490 (.562); | | At3g10500 | |
| | | Caffeate O- | | (.536) | |
| | | methyltransferase | | | |
| | | (COMT), | | | |
| | | At5g54160 (.554) | | | |
| ADT6 | ADT3, At2g27820 | 4CL3, At1g65060 | UGT71B1, | bHLH family | AMT1; 2, At1g64780 |
| Top 100 | (.567); | (.433); CCR1, | At1g24100 | protein, | (.595); |
| r = .595 | Tyrosine | At1g15950 (.433); | (.464); | At4g17880 | NTP3, At3g21670 |
| to .440 | aminotransferase- | CAD4, At3g19450 | UGT78D2^{d,e} | (.526); | (.569); |
| | like, At5g36160 | (.421) | At5g17050 | AP2-domain | Neutral amino acid |
| | (.460) | | (.436); | containing | transmembrane |
| | | | CHI family | protein, | transporter, |
| | | | protein^{f}, | At2g44940 | At1g58360 (.457) |
| | | | At5g05270 | (.494) | |
| | | | (.420) | | |

^{a}The Bio-Array Resource for Plant Functional Genomics (http://bar.utoronto.ca/).
^{b}(Yokoyama et al., 2007).
^{c}(Lim et al., 2001).
^{d}Also known as TRANSPARENT TESTA 6; proposed involvement in anthocyanin pathway (Solfanelli et al., 2006).
^{e}UGT78D2 same as AGT (At5g17050) (Peng et al., 2008).
^{f}(Kubo et al., 2007).
^{g}Also known as TRANSPARENT TESTA 5 (Solfanelli et al., 2006).
^{h}UGT78D2 same as AGT (At5g17050) (Peng et al., 2008).

TABLE 5B
Exemplary genes potentially co-regulated with ADTs involved in primary
metabolic processes (r-values).

| | | Amino Acid | | Nucleotide | DNA/RNA | PPP/Glycolysis/ |
|---|---|---|---|---|---|---|
| Bait | Cell-Division | Biosynthesis | Protein Synthesis | Biosynthesis | Synthesis | Gluconeogenesis |
| ADT1 | SCD1 (Stomatal | Cysteine synthase | tRNA/rRNA | Inosine-5′- | SCABRA 3: | Phosphoglycerate |
| Top 100 | cytokinesis- | D1 (.802); | methyltransferase | mono- | DNA-directed | kinase (.805); |
| r = .959 | defective 1 | DAHP synthase 2 | (SpoU) family | phosphate | RNA | Glucose-6- |
| to .844 | (.867); | (At4g33510) | protein (.875); | dehydrogenase | polymerase | phosphate |
| | Kinesin motor | (.693) | tRNA synthetase | (.797); | (.856); | dehydrogenase |
| | protein-related | | class II (.872); | Thymidine | 40S ribosomal | (.781); |
| | (.865); | | tRNA synthetase | kinase (.749); | protein S19 | 6-phosphofructo- |
| | At1g20570: | | beta subunit | Adenylosuccinate | (RPS19B) | kinase (.772); |
| | tubulin family | | family protein | synthase | (.811); | Pyrophosphate- |
| | protein (.859); | | (.830); | (.747) | Histone H2A 8 | dependent 6- |
| | ATK5: | | Eukaryotic | | (At2g38810) | phosphofructose- |
| | microtubule | | translation | | (.804); | 1-kinase |
| | motor (.749); | | initiation factor, | | H4 histone | (.743); |
| | HINKEL: | | EIF4B5 (.774); | | acetyltransferase | Ribose-5- |
| | microtubule | | 3′-tRNA | | (.775); | phosphate |
| | motor (.669) | | processing | | Histone H2A | adenylyltransferase |
| | | | endoribonuclease | | (At4g27230) | (.727) |
| | | | (.765) | | (.768); | |
| | | | | | NRPD2b: DNA- | |
| | | | | | directed RNA | |
| | | | | | polymerase | |
| | | | | | (.755); | |
| | | | | | 60S ribosomal | |
| | | | | | protein L35a | |
| | | | | | (.750); | |
| | | | | | 50S ribosomal | |
| | | | | | protein L18 | |
| | | | | | family (.747); | |
| | | | | | 60S ribosomal | |
| | | | | | protein L18A | |
| | | | | | (RPL18aC) | |
| | | | | | (.710); | |
| | | | | | 60S ribosomal | |
| | | | | | protein L36 | |
| | | | | | (RPL36C) | |
| | | | | | (.672); | |
| | | | | | POLD4: DNA- | |
| | | | | | directed DNA | |
| | | | | | polymerase | |
| | | | | | (.665); | |
| | | | | | NRPB8A: DNA- | |
| | | | | | directed RNA | |
| | | | | | polymerase | |
| | | | | | (.664) | |
| ADT2 | At2g37080 | Gln synthetase | AT2G40660, | | NRPA2: DNA- | Pyruvate kinase β |
| Top 100 | (Myosin heavy | (GS) 1;5 | putative | | directed RNA | subunit 1 |
| r = .946 | chain-related) | (At1g48470) | methionyl-tRNA | | polymerase | (At5g52920) |
| to .641 | (.889); | (.766); | synthetase | | (.853); | (.856); |
| | Kinesin motor | Glu | (.901); | | RNA | |
| | protein-related | dehydrogenase | PDX2 (pyridoxine | | polymerase I | |
| | (At2g28620) | (At1g51720) | biosynthesis 2); | | specific | |
| | (.818); | (.762); | Glutaminyl-tRNA | | transcription | |
| | CYCA1: cyclin- | Arg biosynthesis | synthase (.724); | | initiation | |
| | dependent | protein ArgJ | Peptidyl-tRNA | | factor RRN3 | |
| | protein kinase | family | hydrolase family | | family protein | |
| | regulator | (At2g37500) | protein | | (.849); | |
| | (.749); | (.708) | (At1g18440) | | ORC2: DNA | |
| | KATB_ATK2: | | (.691) | | replication | |
| | microtubule | | | | origin binding | |
| | binding (.743); | | | | (.827); | |
| | At5g51770 | | | | RNA helicase, | |
| | (protein kinase | | | | putative | |
| | family protein) | | | | (At4g18465) | |
| | (.742); | | | | (.820); | |
| | MAP65-4 | | | | NRPE5: DNA- | |
| | (Microtubule- | | | | directed RNA | |
| | associated | | | | polymerase | |
| | protein) (.704) | | | | (.747); | |
| | | | | | DEAD box RNA | |
| | | | | | helicase | |
| | | | | | (.746); | |
| | | | | | ribosomal | |
| | | | | | protein L27 | |
| | | | | | family protein | |
| | | | | | (.745); | |
| | | | | | Polynucleotide | |
| | | | | | adenylyltransferase | |
| | | | | | family | |
| | | | | | protein (.720) | |

| | | | Nucleotide | DNA/RNA | PPP/Glycolysis/ |
|---|---|---|---|---|---|
| Bait | Amino Acid Biosynthesis | Protein Synthesis | Biosynthesis | Synthesis | Gluconeogenesis |
| ADT3 | ADT6 (.567); | | | Sigma factor A: | Malate |
| Top 100 | AtGLDP2: Gly dehydrogenase | | | DNA-directed | dehydrogenase |
| r = .570 | (.512); | | | RNA | (At1g53240) |
| to .416 | Ser acetyltransferase (.442); | | | polymerase | (.461); |
| | Glu synthase (.426); | | | (.427) | |
| | TYRAAt2 (.423) | | | | |
| ADT4 | Indole-3-glycerol phosphate | tRNA synthetase | | | Glucose-6- |
| Top 100 | synthase (.811); | class I (C) family | | | phosphate |
| r = .811 | AS αS1 (.767); | protein (.531) | | | dehydrogenase |
| to .641 | Trp synthase αS1 (.697); | | | | (.705); |
| | Tyr aminotransferase (.661); | | | | At5g56350 |
| | DHQ synthase (.652); | | | | (Pyruvate |
| | Proline oxidase: (Glu biosynth, Pro | | | | kinase) (.571); |
| | catalysis) (.638); | | | | At5g63680 |
| | DAHP synthetase-like (At1g22410) | | | | (Pyruvate |
| | (.618); | | | | kinase) (.520); |
| | Trp synthase βS1 (.554); | | | | |
| | Glu decarboxylase 3 (.552); | | | | |
| | ADT5 (.551) | | | | |
| ADT5 | DAHP synthetase-like (.651); | | GTP | | Glucose-6- |
| Top 100 | Phosphoglycerate dehydrogenase | | diphospho- | | phosphate |
| r = .714 | (Ser biosynthesis) (.638); | | kinase (.537); | | dehydrogenase |
| to .548 | methionine adenosyltransferase | | UDP-Xylose | | (.663); |
| | (.596); | | synthase 4 | | 6- |
| | PSAT; O-phospho-L-serine:2- | | (.509) | | Phosphogluconate |
| | oxoglutarate aminotransferase | | | | dehydrogenase |
| | (.594); | | | | (.581); |
| | ADT4 (.551); | | | | Isocitrate |
| | AS αS1 (.545); | | | | dehydrogenase |
| | Trp synthase αS1 (.521); | | | | (.522) |
| | Proline oxidase (.516); | | | | |
| | mtLPD2: lipoamide dehydrogenase | | | | |
| | 2 (.515); | | | | |
| | At1g24807 (AS βS-like) (.507) | | | | |
| ADT6 | ADT3 (.567); | | | Sigma factor A; | Ribose-5- |
| Top 100 | Serine acetyltransferase 2;2 (.538); | | | DNA binding/ | phosphate |
| r = .595 | AtGLDP2: Gly dehydrogenase | | | DNA-directed | isomerase |
| to .440 | (.491); | | | RNA | (.485) |
| | HISN1A: ATP phosphoribosyl- | | | polymerase | |
| | transferase (histidine | | | (.441) | |
| | biosynthesis)ⁱ (.466); | | | | |
| | Tyrosine aminotransferase-like | | | | |
| | (At5g36160) (.460) | | | | |

ⁱ(Stepansky and Leustek, 2006).

According to particular aspects, the roles of specific plastid/chloroplast localized ADTs in differentially modulating carbon flux into lignin biogenesis into distinct anatomical regions of the vasculature now provides an exciting opportunity for modulating lignin biosynthesis at the cellular and tissue levels. This is of particular interest given the plastid/chloroplast localization of ADTs, versus the phenylpropanoid enzyme localizations in the cytosol (including membrane associated processes), and cell-wall lignification itself The selective “conscription” of specific ADTs for lignin formation in different cell types likely represents an important evolutionary point during the transition of aquatic plants to a land-based environment.

### Broad Application of the Invention in Modulating in Plants Generally.

According to particular aspects of the present invention, reducing or eliminating expression of one or more arogenate dehydratase (ADT) isoenzymes of the ADT family has substantial utility for regulating (e.g., reducing, decreasing) Phe availability and lignin levels (Phe-derived phenylpropanoids; differential effects on phenylpropanoid metabolism (specifically lignification)) in plants generally, (e.g., vascular plants, hardwood, softwood, graminae, angiosperms, etc.).

While the striking effects on lignin levels demonstrated herein using the exemplary Arabidopsis arogenate dehydratase (ADT) isoenzyme knock outs (KO) are surprising given the different subcellular localizations of ADTs in chloroplasts/plastids for Phe formation versus monolignol pathway enzymes being cytosol/membrane localized (Applicants' data provides the first example of a plastid (chloroplast) associated metabolic process influencing cell-wall formation), they nonetheless represent some of the largest reductions (down to 32% of WT levels) in lignin levels ever reported in genetic manipulations, and was manifested in both vascular bundles and fiber cells.

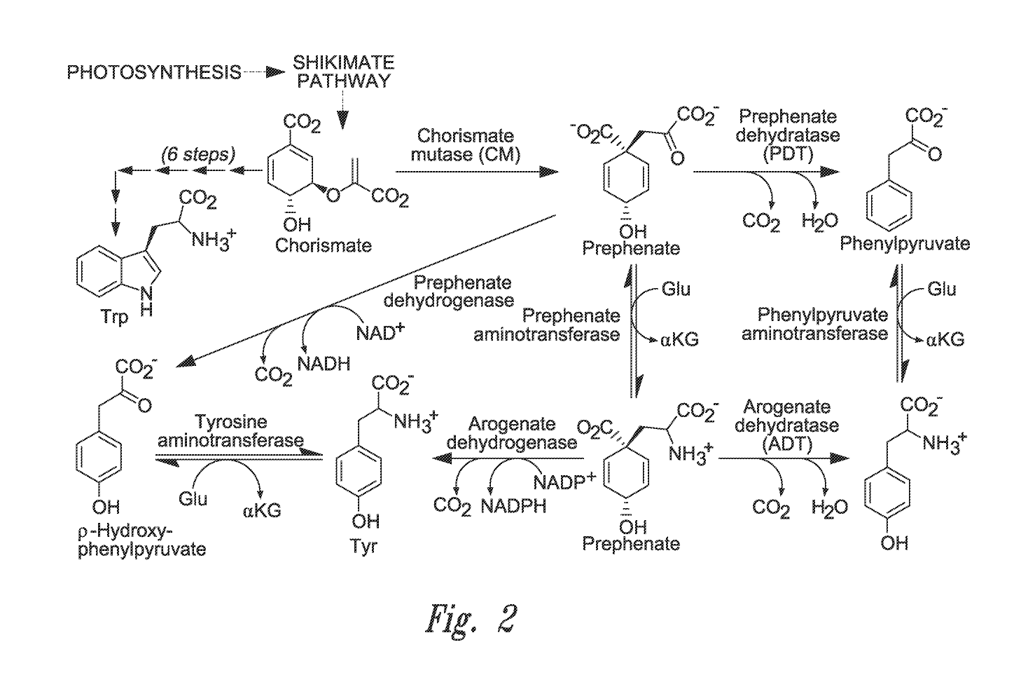

According to additional aspects, said utility is broadly applicable to plants in general based on the fact that: (i) the branch-point position of ADT enzymes in the shikimate-chorismate pathway is widely conserved across plants (e.g., hardwood, softwood, graminae, angiosperms, etc.) (see FIG. 2); and (ii) the plastid (chloroplast) localization of the ADT enzymes is broadly conserved across plants; such that (iii) the linkage between the plastid/chloroplast localized shikimate-chorismate and cytosolic/membrane associated phenylpropanoid metabolic networks would be expected to be broadly conserved across plants; (iv) there was no evidence seen herein for compensatory combinatorial chemistry in lignin, as carbon allocation to the pathway was simply significantly reduced with the resulting vasculature phenotypes due to their decreased lignin levels; and (v) ADT active site sequences that are conserved across plants (see EXAMPLE 16 below).

This conclusion has been further corroborated in Populus trichocarpa (see EXAMPLES 15 and 16 below).

### Plants and Plant Breeding

Particular aspects provide a plant or part thereof, comprising a mutation or genetic modification that modifies the expression or activity of at least one chloroplast-localized arogenate dehydratase (ADT) in one or more cells of the plant, wherein the level, amount, or distribution of lignin is modified (e.g., reduced) relative to that of plants with control or normal expression of the chloroplast-localized ADTs. While the mutation or genetic modification may be any that modifies the ADT expression and/or activity, in preferred aspect, the mutation or genetic modification comprises a mutation of at least one chloroplast-localized arogenate dehydratase (ADT) sequence that modifies the expression or activity thereof in one or more cells of the plant, wherein the level, amount, or distribution of lignin is modified (e.g., reduced) relative to the seed oil of plants with control/normal expression of the ADT(s).

Various plant breeding methods are also useful in establishing useful plant varieties based on such mutations or genetic modifications.

### Plant Breeding

Additional aspects comprise methods for using, in plant breeding, a plant, comprising a mutation that modifies the expression or activity of at least one chloroplast-localized arogenate dehydratase (ADT) (as provided for herein) in one or cells of the plant, wherein the level, amount, or distribution of lignin is modified relative to that of plants with control/normal expression of the ADT(s). One such embodiment is the method of crossing a particular ADT mutant variety with another variety of the plant to form a first generation population of F1 plants. The population of first generation F1 plants produced by this method is also an embodiment of the invention. This first generation population of F1 plants will comprise an essentially complete set of the alleles of the particular PDCT mutant variety. One of ordinary skill in the art can utilize either breeder books or molecular methods to identify a particular F1 plant produced using the particular ADT mutant variety, and any such individual plant is also encompassed by this invention. These embodiments also cover use of transgenic or backcross conversions of particular ADT mutant varieties to produce first generation F 1 plants.

Yet additional aspects comprise a method of developing a particular ADT mutant-progeny plant comprising crossing a particular ADT mutant variety with a second plant and performing a breeding method is also an embodiment of the invention. Given that there are a plurality of chloroplast-localized arogenate dehydratases (ADTs) in a plant cell, breeding to achieve double ADT mutants (two different chloroplast-localized arogenate dehydratase (ADT) mutated in one cell) (or triple, quadruple or higher order ADT mutants comprising mutants of different ADTs) is also encompassed by the invention.

### General Breeding and Selection Methods

Overview.

Plant breeding is the genetic manipulation of plants. The goal of plant breeding is to develop new, unique and superior plant varieties. In practical application of a plant breeding program, and as discussed in more detail herein below, the breeder initially selects and crosses two or more parental lines, followed by repeated ‘selfing’ and selection, producing many new genetic combinations. The breeder can theoretically generate billions of different genetic combinations via crossing, ‘selfing’ and naturally induced mutations. The breeder has no direct control at the cellular level, and two breeders will never, therefore, develop exactly the same line. Each year, the plant breeder selects the germplasm to advance to the next generation. This germplasm may be grown under unique and different geographical, climatic and soil conditions, and further selections may be made during and at the end of the growing season.

Proper testing can detect major faults and establish the level of superiority or improvement over current varieties. In addition to showing superior performance, it is desirable that this is a desired trait(s) for a new variety. The new variety should optimally be compatible with industry standards, or create a new market. The introduction of a new variety may incur additional costs to the seed producer, the grower, processor and consumer, for special advertising and marketing, altered seed and commercial production practices, and new product utilization. The testing preceding release of a new variety should take into consideration research and development costs as well as technical superiority of the final variety. Ideally, it should also be feasible to produce seed easily and economically.

The term ‘homozygous plant’ is hereby defined, with respect to a given locus, as a plant with homozygous genes at 95% or more of its loci.

The term “inbred” as used herein refers, with respect to a given locus, to a homozygous plant or a collection of homozygous plants.

Choice of Breeding or Selection Methods.

Choice of breeding or selection methods depends on the mode of plant reproduction, the heritability of the trait(s) being improved, and the type of variety used commercially (e.g., F1 hybrid variety, pure-line variety, etc.). For highly heritable traits, a choice of superior individual plants evaluated at a single location will be effective, whereas for traits with low heritability, selection should be based on mean values obtained from replicated evaluations of families of related plants. The complexity of inheritance also influences choice of the breeding method. Breeding generally starts with cross-hybridizing two genotypes (a “breeding cross”), each of which may have one or more desirable characteristics that is lacking in the other or which complements the other. If the two original parents do not provide all the desired characteristics, other sources can be included by making more crosses. In each successive filial generation (e.g., F1→F2; F2→F3; F3→F4; F4→F5, etc.), plants are ‘selfed’ to increase the homozygosity of the line. Typically in a breeding program five or more generations of selection and ‘selfing’ are practiced to obtain a homozygous plant. Each plant breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful varieties produced per unit of input (e.g., per year, per dollar expended, etc.).

### Backcross Conversion

An additional embodiment comprises, or is a backcross conversion of a desired plant, comprising a mutation that modifies the expression or activity of at least one chloroplast-localized arogenate dehydratase (ADT) (as provided for herein) in one or more cells of the plant, wherein the level, amount, or distribution of lignin is modified (e.g., reduced) relative to that of plants with control/normal expression of the ADT(s). A backcross conversion occurs when DNA sequences are introduced through traditional (non-transformation) breeding techniques, such as backcrossing. DNA sequences, whether naturally occurring or transgenes, may be introduced using these traditional breeding techniques. Desired traits transferred through this process include, but are not limited to nutritional enhancements, industrial enhancements, reduced lignin, disease resistance, insect resistance, herbicide resistance, agronomic enhancements, grain quality enhancement, waxy starch, breeding enhancements, seed production enhancements, and male sterility. A further embodiment comprises or is a method of developing a backcross conversion plant that involves the repeated backcrossing to such ADT mutation(s). The number of backcrosses made may be 2, 3, 4, 5, 6 or greater, and the specific number of backcrosses used will depend upon the genetics of the donor parent and whether molecular markers are utilized in the backcrossing program.

### Essentially Derived Varieties

Another embodiment of the invention is an essentially derived variety of a plant, comprising a mutation that modifies the expression or activity of at least one chloroplast-localized arogenate dehydratase (ADT) (as provided for herein) in one or more cells of the plant, wherein the level, amount, or distribution of lignin is modified (e.g., reduced) relative to that of plants with control/normal expression of the ADT(s). As determined by the UPOV Convention, essentially derived varieties may be obtained for example by the selection of a natural or induced mutant, or of a somaclonal variant, the selection of a variant individual from plants of the initial variety, backcrossing, or transformation by genetic engineering. An essentially derived variety of such ADT mutants is further defined as one whose production requires the repeated use thereof, or is predominately derived from genotype of a particular ADT mutant(s). International Convention for the Protection of New Varieties of Plants, as amended on Mar. 19, 1991, Chapter V, Article 14, Section 5(c).

### DNA Constructs

The present invention also contemplates the fabrication of DNA constructs (e.g., expression vectors, recombination vectors, anti-sense constructs, RNAi, siRNA constructs, etc.) comprising the isolated nucleic acid sequence containing the genetic element and/or coding sequence from the disclosed ADT mutant varieties operatively linked to plant gene expression control sequences. “DNA constructs” are defined herein to be constructed (not naturally-occurring) DNA molecules useful for introducing DNA into host cells, and the term includes chimeric genes, expression cassettes, and vectors.

As used herein “operatively linked” refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N.Y., 1982; and Sambrook et al., Molecular Cloning: A Laboratory Manual, Cold Spring Harbor, N.Y., 1989.

“Expression control sequences” are DNA sequences involved in any way in the control of transcription or translation. Suitable expression control sequences and methods of making and using them are well known in the art.

The expression control sequences preferably include a promoter. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-2361, 1987. Also, the location of the promoter relative to the transcription start may be optimized See, e.g., Roberts et al., Proc. Natl. Acad. Sci. USA, 76:760-764, 1979.

Many suitable promoters for use in plants are well known in the art. For instance, suitable constitutive promoters for use in plants include the promoters of plant viruses, such as the peanut chlorotic streak caulimovirus (PC1SV) promoter (U.S. Pat. No. 5,850,019); the 35S and 19S promoter from cauliflower mosaic virus (CaMV) (Odell et al., 1313:3810-812, 1985); promoters of the Chlorella virus methyltransferase genes (U.S. Pat. No. 5,563,328); the full-length transcript promoter from figwort mosaic virus (FMV) (U.S. Pat. No. 5,378,619); the promoters from such genes as rice actin (McElroy et al., Plant Cell 2:163-171 (1990)), ubiquitin (Christiansen et al., Plant Mol. Biol. 12:619-632, 1989), and (Christiansen et al., Plant Mol. Biol. 18: 675-689, 1992), pEMU (Last et al., Theor. Appl. Genet. 81:581-588, 1991), MAS (Velten et al., Embo J. 3:2723-2730, 1984), wheat histone (Lepetit et al., Mol. Gen. Genet. 231:276-285, 1992), and Atanassova et al., Plant Journal 2:291-300, 1992), Brassica napus ALS3 (International Publication No. WO 97/41228); and promoters of various Agrobacterium genes (see U.S. Pat. Nos. 4,771,002; 5,102,796; 5,182,200; and 5,428,147).

Suitable inducible promoters for use in plants include: the promoter from the ACE1 system which responds to copper (Mett et al., Proc. Natl. Acad. Sci. 90:4567-4571, 1993): the promoter of the wheat In 2 gene which responds to benzenesulfonomide herbicide safeners (U.S. Pat. No. 5,364,780 and Gatz et al., Mol. Gen. Genet. 243:32-38, 1994), and the promoter of the Tet repressor from Tn10 (Gatz et al., Mol. Gen. Genet. 227:229-237, 1991). According to one embodiment, the promoter for use in plants is one that responds to an inducing agent to which plants normally do not respond. An exemplary inducible promoter of this type is the inducible promoter from a steroid hormone gene, the transcriptional activity of which is induced by a glucosteroid hormone (Schena et al., Proc. Natl. Acad. Sci. 88:10421, 1991) or the application of a chimeric transcription activator, XVE, for use in an estrogen receptor-based inducible plant expression system activated by estradiol (Zou et al., Plant J. 24 265-273, 2000). Other inducible promoters for use in plants are described in European Patent No. 332104, International Publication No. WO 93/21334 and International Publication No. WO 97/06269, and discussed in Gatz and Lenk Trends Plant Sci., 3:352-358, 1998, and Zou and Chua, Curr. Opin. Biotechnol., 11:146-151, 2000. Finally, promoters composed of portions of other promoters and partially or totally synthetic promoters can be used. See, e.g., Ni et al., Plant J. 7:661-676, 1995, and International Publication No. WO 95/14098, which describes such promoters for use in plants.

The promoter may include, or be modified to include, one or more enhancer elements. Preferably, the promoter will include a plurality of enhancer elements. Promoters containing enhancer elements provide for higher levels of transcription as compared to promoters that do not include them. Suitable enhancer elements for use in plants include the PC1SV enhancer element (U.S. Pat. No. 5,850,019), the CaMV 35S enhancer element (U.S. Pat. Nos. 5,106,739 and 5,164,316), and the FMV enhancer element (Maiti et al., Transgenic Res., 6:143-156, 1997). See also, International Publication No. WO 96/23898 and Enhancers and Eukaryotic Expression (Cold Spring Harbor Press, Cold Spring Harbor, N.Y., 1983).

For efficient expression, the coding sequences are preferably also operatively linked to a 3′ untranslated sequence. The 3′ untranslated sequence will preferably include a transcription termination sequence and a polyadenylation sequence. The 3′ untranslated region can be obtained from the flanking regions of genes from Agrobacterium, plant viruses, plants and other eukaryotes. Suitable 3′ untranslated sequences for use in plants include those of the cauliflower mosaic virus 35S gene, the phaseolin seed storage protein gene, the pea ribulose-1,5-bisphosphate carboxylase small subunit E9 gene, the wheat 7S storage protein gene, the octopine synthase gene, and the nopaline synthase gene.

A 5′ untranslated leader sequence can also be optionally employed. The 5′ untranslated leader sequence is the portion of an mRNA that extends from the 5′ CAP site to the translation initiation codon. This region of the mRNA is necessary for translation initiation in plants and plays a role in the regulation of gene expression. Suitable 5′ untranslated leader sequence for use in plants includes those of alfalfa mosaic virus, cucumber mosaic virus coat protein gene, and tobacco mosaic virus.

The DNA construct may be a ‘vector.’ The vector may contain one or more replication systems which allow it to replicate in host cells. Self-replicating vectors include plasmids, cosmids and virus vectors. Alternatively, the vector may be an integrating vector which allows the integration into the host cell's chromosome of the DNA sequence encoding the root-rot resistance gene product. The vector desirably also has unique restriction sites for the insertion of DNA sequences. If a vector does not have unique restriction sites it may be modified to introduce or eliminate restriction sites to make it more suitable for further manipulation.

Vectors suitable for use in expressing the nucleic acids, which when expressed in a plant modulate the expression or activity of at least one phosphatidylcholine:diacylglycerol cholinephosphotransferase (PDCT) (as provided for herein) in one or more seeds or developing seeds of the plant, wherein the level, amount, or distribution of fatty acid unsaturation in the seed oil is modified relative to the seed oil of plants with normal seed expression of the PDCT, include but are not limited to pMON979, pMON977, pMON886, pCaMVCN, and vectors derived from the tumor inducing (Ti) plasmid of Agrobacterium tumefaciens described by Rogers et al., Meth. Enzymol., 153:253-277, 1987. The nucleic acid is inserted into the vector such that it is operably linked to a suitable plant active promoter. Suitable plant active promoters for use with the nucleic acids include, but are not limited to CaMV35S, ACTIN, FMV35S, NOS and PCSLV promoters. The vectors comprising the nucleic acid can be inserted into a plant cell using a variety of known methods. For example, DNA transformation of plant cells include but are not limited to Agrobacterium-mediated plant transformation, protoplast transformation, electroporation, gene transfer into pollen, injection into reproductive organs, injection into immature embryos and particle bombardment. These methods are described more fully in U.S. Pat. No. 5,756,290, and in a particularly efficient protocol for wheat described in U.S. Pat. No. 6,153,812, and the references cited therein. Site-specific recombination systems can also be employed to reduce the copy number and random integration of the nucleic acid into the plant genome. For example, the Cre/lox system can be used to immediate lox site-specific recombination in plant cells. This method can be found at least in Choi et al., Nuc. Acids Res. 28:B19, 2000).

### Transgenes:

Molecular biological techniques allow the isolation and characterization of genetic elements with specific functions, such as encoding specific protein products. Scientists in the field of plant biology developed a strong interest in engineering the genome of plants to contain and express foreign genetic elements, or additional, or modified versions of native or endogenous genetic elements in order to alter the traits of a plant in a specific manner. Any DNA sequences, whether from a different species or from the same species, which are inserted into the genome using transformation are referred to herein collectively as “transgenes.” Several methods for producing transgenic plants have been developed, and the present invention, in particular embodiments, also relates to transformed versions of the genotypes of the invention and/or transformed versions comprising one or more transgenes modify directly or indirectly the expression or activity of at least one chloroplast-localized arogenate dehydratase (ADT) (as provided for herein) in one or more cells of the plant, wherein the level, amount, or distribution of lignin is modified (e.g., reduced) relative to that of plants with control/normal expression of the ADT(s).

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

The most prevalent types of plant transformation involve the construction of an expression vector. Such a vector comprises a DNA sequence that contains a gene under the control of or operatively linked to a regulatory element, for example a promoter. The vector may contain one or more genes and one or more regulatory elements. Various genetic elements can be introduced into the plant genome using transformation. These elements include but are not limited to genes; coding sequences (in sense or anti-sense orientation); inducible, constitutive, and tissue specific promoters; enhancing sequences; and signal and targeting sequences.

A genetic trait which has been engineered into a particular plant using transformation techniques could be moved into another line using traditional breeding techniques that are well known in the plant breeding arts. For example, a backcrossing approach could be used to move a transgene (e.g., including a chromosomal gene mutation or knock out) from a transformed oil seed-bearing plant to an elite plant variety and the resulting progeny would comprise a transgene (e.g., or knock out as the case may be). As used herein, “crossing” can refer to a simple X by Y cross, or the process of backcrossing, depending on the context. The term “breeding cross” excludes the processes of selfing or sibbing.

With transgenic plants according to the present invention, a foreign protein and/or and modified expression of an endogenous protein or product (e.g., Phe-derived phenylpropanoids) can be commercially implemented. Thus, techniques for the selection and propagation of transformed plants, which are well understood in the art, yield a plurality of transgenic or recombinant plants which are harvested in a conventional manner, and a plant product can then can be extracted from a tissue of interest or from total biomass. For example, protein and oil extraction from plant biomass can be accomplished by known methods which are discussed, for example, by Heney and Orr, Anal. Biochem. 114:92-96, 1981.

According to a preferred embodiment, the recombinant/transgenic plant provided for commercial production is essentially any plant, including a vascular plant (e.g., at least one of Arabidopsis, poplar, Populus trichocarpa, pine, Pinus taeda, rice, Oryza sativa, vitis vinifera, canola (e.g., Brassica napus or B. rapa), soybean (e.g., Glycine max), or sunflower (e.g., Helianthus annuus). In another preferred embodiment, the biomass of interest is one having reduced lignin. A genetic map can be generated, primarily via conventional RFLP, PCR, and SSR analysis, which identifies the approximate chromosomal location of the integrated DNA molecule. For exemplary methodologies in this regard, see Glick and Thompson, Methods in Plant Molecular Biology and Biotechnology 269-284 (CRC Press, Boca Raton, 1993). Map information concerning chromosomal location is useful for proprietary protection of a subject recombinant/transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

### Introduction of Transgenes of Agronomic Interest by Transformation

Agronomic genes can be expressed in transformed plants. For example, plants can be genetically engineered to express various phenotypes of agronomic interest, or, alternatively, transgenes can be introduced into a plant by breeding with a plant that has the transgene. Through the transformation of plant, the expression of genes can be modulated to enhance disease resistance, insect resistance, herbicide resistance, water stress tolerance and agronomic traits, lignin content (as disclosed herein), as well as seed quality traits. Transformation can also be used to insert DNA sequences which provide gene knock outs (KO), or which control or help control, for example male-sterility or some other desirable trait. DNA sequences native to particular plants as well as non-native DNA sequences can be transformed and used to modulate levels of native or non-native proteins. Anti-sense technology, RNAi, siRNA technology, various promoters, targeting sequences, enhancing sequences, and other DNA sequences can be inserted into the particular genome for the purpose of modulating the expression of proteins. Many exemplary genes implicated in this regard are known in the art.

### Variants of Chloroplast-Localized Arogenate Dehydratase (ADT) Nucleic Acids and Proteins

As used herein, a “biological activity” refers to a function of a polypeptide including but not limited to complexation, dimerization, multimerization, substrate binding, receptor-associated ligand binding and/or endocytosis, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and metabolism. A biological activity can be assessed by assays described herein and by any suitable assays known to those of skill in the art, including, but not limited to in vitro assays, including cell-based assays, in vivo assays, including assays in animal models for particular diseases.

In particular exemplary aspects, the chloroplast-localized ADTs, or variants thereof comprise an amino acid sequence as disclosed herein, having from 1, to about 3, to about 5, to about 10, or to about 20 conservative amino acid substitutions), or a fragment of an ADT sequence having from 1, to about 3, to about 5, to about 10, or to about 20 conservative amino acid substitutions). In certain aspects, chloroplast-localized ADTs or variants thereof, comprises a non-conservative or a conservative amino acid substitution variant thereof.

Functional phosphatidylcholine:diacylglycerol cholinephosphotransferase (PDCT), variants are those proteins that display (or lack) one or more of the biological activities of phosphatidylcholine:diacylglycerol cholinephosphotransferase (PDCT).

As used herein, the term “wild type” chloroplast-localized ADT, means a naturally occurring chloroplast-localized ADTs allele found within plants which encodes a functional chloroplast-localized ADTs protein. In contrast, the term “mutant” chloroplast-localized ADT. as used herein, refers to a chloroplast-localized ADT allele, which does not encode a functional ROD1 or PDCT protein (e.g., a knock out, insertion, or a chloroplast-localized ADT allele that is knocked out or silent, or encoding a non-functional chloroplast-localized ADT protein, which, as used herein, refers to a chloroplast-localized ADT protein having no biological activity or a significantly reduced biological activity as compared to the corresponding wild-type functional chloroplast-localized ADT protein, or encoding no chloroplast-localized ADT protein at all. Such a “mutant chloroplast-localized ADT allele” (also called “full knock-out” or “null” allele) is a wild-type chloroplast-localized ADT allele, which comprises one or more mutations in its nucleic acid sequence, whereby the mutation(s) preferably result in a significantly reduced (absolute or relative) amount of functional chloroplast-localized ADT protein in the cell in vivo. Exemplary mutant alleles of the chloroplast-localized ADT protein-encoding nucleic acid sequences are disclosed herein. Mutant alleles can be either “natural mutant” alleles, which are mutant alleles found in nature (e.g. produced spontaneously without human application of mutagens) or induced mutant” alleles, which are induced by human intervention, e.g. by mutagenesis.

Variants of chloroplast-localized ADTs have utility for aspects of the present invention. Variants can be naturally or non-naturally occurring. Naturally occurring variants (e.g., polymorphisms, paralogs, homologs, orthologs) are found in various species and comprise amino acid sequences which are substantially identical to the chloroplast-localized ADT amino acid sequences disclosed herein. Species homologs (e.g., orthologs) of the protein can be obtained using subgenomic polynucleotides of the invention, as described below, to make suitable probes or primers for screening cDNA expression libraries from other plant species, which encode homologs/paralogs/orthologs of the protein, and expressing the cDNAs as is known in the art. For example, orthologs (and orthologous knock outs) are provided for herein.

Non-naturally occurring variants which retain (or lack) substantially the same biological activities as naturally occurring protein variants are also included herein. In particular aspects, naturally or non-naturally occurring variants have amino acid sequences which are at least 50%, 85%, 90%, 95%, 96%, 97%, 98%, 99% or greater than 99% identical to the chloroplast-localized ADT amino acid sequences shown herein. In certain aspects, the molecules are at least 85%, 90%, 98%, 99% or greater than 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 2:

TABLE 2
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus In addition, the phrase “amino acid residue” is defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. In certain aspects, amino acid changes in the chloroplast-localized ADT polypeptide variants are non-conservative amino acid changes, i.e., substitutions of non-similarly charged or uncharged amino acids, and/or include insertions and deletions. In certain aspects, changes are conservative.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Properties and functions of chloroplast-localized ADT polypeptide protein or polypeptide variants are of different or the same type as a protein comprising the amino acid sequence encoded by the chloroplast-localized ADT nucleotide sequences disclosed herein, although the properties and functions of variants can differ in degree.

Variants of the chloroplast-localized ADT polypeptides disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do or do not affect functional activity of the proteins are also variants. Covalent variants can be prepared by linking functionalities to groups which are found in the amino acid chain or at the N- or C-terminal residue, as is known in the art.

A subset of mutants, called muteins, is a group of polypeptides in which neutral amino acids, such as serines, are substituted for cysteine residues which do not participate in disulfide bonds. These mutants may be stable over a broader temperature range than native secreted proteins (see, e.g., Mark et al., U.S. Pat. No. 4,959,314).

It will be recognized in the art that some amino acid sequences of the chloroplast-localized ADT polypeptides of the invention can be varied to provide a significant effect on the structure or function of the protein. If such differences in sequence are contemplated, it should be remembered that there may be particular or critical areas on the protein which determine activity as disclosed herein. In general, it is possible to replace residues that form the tertiary structure, provided that residues performing a similar or different function are used, as desired. In other instances, the type of residue may be completely unimportant if the alteration occurs at a non-critical region of the protein. The replacement of amino acids can also change the selectivity of ligand binding (Ostade et al., Nature 361:266-268, 1993). Thus, the chloroplast-localized ADT polypeptides of the present invention may include one or more amino acid substitutions, deletions or additions, either from natural mutations or human manipulation.

Amino acids in the chloroplast-localized ADT polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for substrate ligand binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes in particular aspects are preferably of a major nature, such as insertions, deletions or non-conservative amino acid substitutions that significantly affect the folding or activity of the protein (to provide for reduced chloroplast-localized ADT activity). Of course, the number of amino acid substitutions a skilled artisan would make depends on many factors, including those described above. Other embodiments comprise conservative substitutions. Generally speaking, the number of substitutions for any given phosphatidylcholine:diacylglycerol cholinephosphotransferase (PDCT) polypeptide will not be more than 50, 40, 30, 25, 20, 15, 10, 5 or 3.

### Fusion Proteins

Fusion proteins comprising proteins or polypeptide fragments of chloroplast-localized ADT polypeptide can also be constructed. Fusion proteins are useful for generating antibodies against amino acid sequences and for use in various targeting and assay systems. For example, fusion proteins can be used to identify proteins which interact with a chloroplast-localized ADT polypeptide of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence can be used.

A fusion protein comprises two protein segments fused together by means of a peptide bond. Amino acid sequences for use in fusion proteins of the invention can, for example, utilize the chloroplast-localized ADT amino acid sequences shown herein, or can be prepared from chloroplast-localized ADT variants, such as those described herein. The first protein segment can include of a full-length chloroplast-localized ADT polypeptide. Other first protein segments can consist of portions of a chloroplast-localized ADT.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and virus protein fusions.

These fusions can be made, for example, by covalently linking two protein segments or by standard procedures in the art of molecular biology. Recombinant DNA methods can be used to prepare fusion proteins, for example, by making a DNA construct which comprises a coding region for the chloroplast-localized ADT protein sequence in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Nucleic Acid Sequences Encoding Mutant Chloroplast-Localized Arogenate Dehydratase (ADT) Proteins

Nucleic acid sequences comprising one or more nucleotide deletions, insertions or substitutions relative to the wild type nucleic acid sequences are another embodiment of the invention, as are fragments of such mutant nucleic acid molecules. Such mutant nucleic acid sequences (referred to as chloroplast-localized arogenate dehydratase (ADT) sequences) can be generated and/or identified using various known methods, including as described further below. Again, such nucleic acid molecules are provided both in endogenous form and in isolated form. In one embodiment, the mutation(s) result in one or more changes (deletions, insertions and/or substitutions) in the amino acid sequence of the encoded chloroplast-localized ADT protein (i.e. it is not a “silent mutation”). In another embodiment, the mutation(s) in the nucleic acid sequence result in a significantly reduced or completely abolished biological activity of the encoded chloroplast-localized ADT protein relative to the wild type protein.

The nucleic acid molecules may, thus, comprise one or more mutations, such as:

(a) a “missense mutation”, which is a change in the nucleic acid sequence that results in the substitution of an amino acid for another amino acid;

(b) a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.

(c) an “insertion mutation” of one or more amino acids, due to one or more codons having been added in the coding sequence of the nucleic acid;

(d) a “deletion mutation” of one or more amino acids, due to one or more codons having been deleted in the coding sequence of the nucleic acid;

(e) a “frameshift mutation”, resulting in the nucleic acid sequence being translated in a different frame downstream of the mutation. A frameshift mutation can have various causes, such as the insertion, deletion or duplication of one or more nucleotides.

It is desired that the mutation(s) in the nucleic acid sequence preferably result in a mutant protein comprising significantly reduced or no biological activity in vivo or in the production of no protein. In certain aspects, a mutation which results in a protein comprising at least one amino acid insertion, deletion and/or substitution relative to the wild type protein can lead to significantly reduced or no biological activity. It is, however, understood that mutations in certain parts of the protein are more likely to result in a reduced function of the mutant chloroplast-localized ADT protein, such as mutations leading to truncated proteins, whereby significant portions of the functional domains are lacking.

Thus in one embodiment, nucleic acid sequences comprising one or more of any of the types of mutations described above are provided. In another embodiment, chloroplast-localized ADT sequences comprising one or more stop codon (nonsense) mutations, one or more missense mutations and/or one or more frameshift mutations are provided. Any of the above mutant nucleic acid sequences are provided per se (in isolated form), as are plants and plant parts comprising such sequences endogenously.

A nonsense mutation in an chloroplast-localized ADT allele, as used herein, is a mutation in an chloroplast-localized ADT allele whereby one or more translation stop codons are introduced into the coding DNA and the corresponding mRNA sequence of the corresponding wild type chloroplast-localized ADT allele. Translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. In one embodiment, a mutant chloroplast-localized ADT allele comprising a nonsense mutation is a chloroplast-localized ADT allele wherein an in-frame stop codon is introduced in the chloroplast-localized ADT codon sequence by a single nucleotide substitution, such as the mutation of CAG to TAG, TGG to TAG, TGG to TGA, or CAA to TAA. In another embodiment, a mutant chloroplast-localized ADT allele comprising a nonsense mutation is a chloroplast-localized ADT allele wherein an in-frame stop codon is introduced in the chloroplast-localized ADT codon sequence by double nucleotide substitutions, such as the mutation of CAG to TAA, TGG to TAA, or CGG to TAG or TGA. In yet another embodiment, a mutant chloroplast-localized ADT allele comprising a nonsense mutation is a chloroplast-localized ADT allele wherein an in-frame stop codon is introduced in the chloroplast-localized ADT codon sequence by triple nucleotide substitutions, such as the mutation of CGG to TAA. The truncated protein lacks the amino acids encoded by the coding DNA downstream of the mutation (i.e. the C-terminal part of the chloroplast-localized ADT protein) and maintains the amino acids encoded by the coding DNA upstream of the mutation (i.e. the N-terminal part of the chloroplast-localized ADT protein).

Obviously, mutations are not limited to the ones disclosed herein, and it is understood that analogous STOP mutations may be present in chloroplast-localized ADT alleles other than those examples depicted and referred to herein.

A missense mutation in an chloroplast-localized ADT allele, as used herein, is any mutation (deletion, insertion or substitution) in a chloroplast-localized ADT allele whereby one or more codons are changed in the coding DNA and the corresponding mRNA sequence of the corresponding wild type chloroplast-localized ADT allele, resulting in the substitution of one or more amino acids in the wild type chloroplast-localized ADT protein for one or more other amino acids in the mutant chloroplast-localized ADT protein.

A frameshift mutation in an chloroplast-localized ADT allele, as used herein, is a mutation (deletion, insertion, duplication, and the like) in a chloroplast-localized ADT allele that results in the nucleic acid sequence being translated in a different frame downstream of the mutation.

### Downregulation of Chloroplast-Localized ADT(s):

Several methods are available in the art to produce a silencing RNA molecule, i.e. an RNA molecule which when expressed reduces the expression of a particular gene or group of genes, including the so-called “sense” or “antisense” RNA technologies.

Antisense Technology.

Thus in one embodiment, the inhibitory RNA molecule encoding chimeric gene is based on the so-called antisense technology. In other words, the coding region of the chimeric gene comprises a nucleotide sequence of at least 19 or 20 consecutive nucleotides of the complement of the nucleotide sequence of the chloroplast-localized ADT or an orthologue thereof. Such a chimeric gene may be constructed by operably linking a DNA fragment comprising at least 19 or 20 nucleotides from ROD1 encoding gene or an orthologue thereof, isolated or identified as described elsewhere in this application, in inverse orientation to a plant expressible promoter and 3′ end formation region involved in transcription termination and polyadenylation.

Co-Suppression Technology.

In another embodiment, the inhibitory RNA molecule encoding chimeric gene is based on the so-called co-suppression technology. In other words, the coding region of the chimeric gene comprises a nucleotide sequence of at least 19 or 20 consecutive nucleotides of the nucleotide sequence of the chloroplast-localized ADT encoding gene or an ortholog thereof. Such a chimeric gene may be constructed by operably linking a DNA fragment comprising at least 19 or 20 nucleotides from the chloroplast-localized ADT encoding gene or an ortholog thereof, in direct orientation to a plant expressible promoter and 3′ end formation region involved in transcription termination and polyadenylation.

The efficiency of the above mentioned chimeric genes in reducing the expression of the chloroplast-localized ADT encoding gene or an ortholog thereof may be further enhanced by the inclusion of DNA element which result in the expression of aberrant, unpolyadenylated inhibitory RNA molecules or results in the retention of the inhibitory RNA molecules in the nucleus of the cells. One such DNA element suitable for that purpose is a DNA region encoding a self-splicing ribozyme, as described in WO 00/01133 (incorporated herein by reference in its entirety and particularly for its teachings on self-splicing ribozymes). Another such DNA element suitable for that purpose is a DNA region encoding an RNA nuclear localization or retention signal, as described in PCT/AU03/00292 published as WO03/076619 (incorporated by reference).

Double-Stranded RNA (dsRNA) or Interfering RNA (RNAi).

A convenient and very efficient way of downregulating the expression of a gene of interest uses so-called double-stranded RNA (dsRNA) or interfering RNA (RNAi), as described e.g. in WO99/53050 (incorporated herein by reference in its entirety and particularly for its teachings on RNAi)). In this technology, an RNA molecule is introduced into a plant cell, whereby the RNA molecule is capable of forming a double stranded RNA region over at least about 19 to about 21 nucleotides, and whereby one of the strands of this double stranded RNA region is about identical in nucleotide sequence to the target gene (“sense region”), whereas the other strand is about identical in nucleotide sequence to the complement of the target gene or of the sense region (“antisense region”). It is expected that for silencing of the target gene expression, the nucleotide sequence of the 19 consecutive nucleotide sequences may have one mismatch, or the sense and antisense region may differ in one nucleotide. To achieve the construction of such RNA molecules or the encoding chimeric genes, use can be made of the vector as described in WO 02/059294.

Thus, in one embodiment of the invention, a method for regulating (e.g., reducing) lignin in plants or cells thereof is provided comprising the step of introducing a chimeric gene into a cell of the plant, wherein the chimeric gene comprises the following operably linked DNA elements:
(a) a plant expressible promoter;
(b) a transcribed DNA region, which when transcribed yields a double-stranded RNA molecule capable of reducing specifically the expression of chloroplast-localized ADT or an ortholog thereof, and the RNA molecule comprising a first and second RNA region wherein
   i) the first RNA region comprises a nucleotide sequence of at least 19 consecutive nucleotides having at least about 94% sequence identity to the nucleotide sequence of chloroplast-localized ADT or of an ortholog thereof;
   ii) the second RNA region comprises a nucleotide sequence complementary to the at least 19 consecutive nucleotides of the first RNA region;
   iii) the first and second RNA region are capable of base-pairing to form a double stranded RNA molecule between at least the 19 consecutive nucleotides of the first and second region; and
(c) a 3′ end region comprising transcription termination and polyadenylation signals functioning in cells of the plant.

The length of the first or second RNA region (sense or antisense region) may vary from about 19 nucleotides (nt) up to a length equaling the length (in nucleotides) of the endogenous gene involved in callose removal. The total length of the sense or antisense nucleotide sequence may thus be at least at least 25 nt, or at least about 50 nt, or at least about 100 nt, or at least about 150 nt, or at least about 200 nt, or at least about 500 nt. It is expected that there is no upper limit to the total length of the sense or the antisense nucleotide sequence. However for practical reasons (such as e.g. stability of the chimeric genes) it is expected that the length of the sense or antisense nucleotide sequence should not exceed 5000 nt, particularly should not exceed 2500 nt and could be limited to about 1000 nt or about 500 nt.

It will be appreciated that the longer the total length of the sense or antisense region, the less stringent the requirements for sequence identity between these regions and the corresponding sequence in chloroplast-localized ADT gene and orthologs or their complements. Preferably, the nucleic acid of interest should have a sequence identity of at least about 75% with the corresponding target sequence, particularly at least about 80%, more particularly at least about 85%, quite particularly about 90%, especially about 95%, more especially about 100%, quite especially be identical to the corresponding part of the target sequence or its complement. However, it is preferred that the nucleic acid of interest always includes a sequence of about 19 consecutive nucleotides, particularly about 25 nt, more particularly about 50 nt, especially about 100 nt, quite especially about 150 nt with 100% sequence identity to the corresponding part of the target nucleic acid. Preferably, for calculating the sequence identity and designing the corresponding sense or antisense sequence, the number of gaps should be minimized, particularly for the shorter sense sequences.

dsRNA encoding chimeric genes according to the invention may comprise an intron, such as a heterologous intron, located e.g. in the spacer sequence between the sense and antisense RNA regions in accordance with the disclosure of WO 99/53050 (incorporated herein by reference).

Synthetic Micro-RNAs (miRNAs).

The use of synthetic micro-RNAs to down-regulate expression of a particular gene in a plant cell, provides for very high sequence specificity of the target gene, and thus allows conveniently to discriminate between closely related alleles as target genes the expression of which is to be down-regulated.

Thus, in another embodiment of the invention, the biologically active RNA or silencing RNA or inhibitory RNA molecule may be a microRNA molecule, designed, synthesized and/or modulated to target and cause the cleavage chloroplast-localized ADT encoding gene or an ortholog thereof in a plant. Various methods have been described to generate and use miRNAs for a specific target gene (including but not limited to Schwab et al. (2006, Plant Cell, 18(5):1121-1133), WO2006/044322, WO2005/047505, EP 06009836, all incorporated herein by reference in their entirety, and particularly for their respective teachings relating to miRNA). Usually, an existing miRNA scaffold is modified in the target gene recognizing portion so that the generated miRNA now guides the RISC complex to cleave the RNA molecules transcribed from the target nucleic acid. miRNA scaffolds could be modified or synthesized such that the miRNA now comprises 21 consecutive nucleotides of the chloroplast-localized ADT encoding nucleotide sequence or an ortholog thereof, such as the sequences represented in the Sequence Listing, and allowing mismatches according to the herein below described rules.

Thus, in one embodiment, the invention provides a method for regulation of lignin in plants or cells thereof comprising the steps of:
a. Introducing a chimeric gene into cells of a plant, said chimeric gene comprising the following operably linked DNA regions:
   i. a plant expressible promoter;
   ii. a DNA region which upon introduction and transcription in a plant cell is processed into a miRNA, whereby the miRNA is capable of recognizing and guiding the cleavage of the mRNA of a chloroplast-localized ADT encoding gene or an ortholog thereof of the plant; and
   iii. optionally, a 3′ DNA region involved in transcription termination and polyadenylation.

The mentioned DNA region processed into a miRNA may comprise a nucleotide sequence which is essentially complementary to a nucleotide sequence of at least 21 consecutive nucleotides of a chloroplast-localized ADT encoding gene or ortholog, provided that one or more of the following mismatches are allowed:
a. A mismatch between the nucleotide at the 5′ end of the miRNA and the corresponding nucleotide sequence in the RNA molecule;
b. A mismatch between any one of the nucleotides in position 1 to position 9 of the miRNA and the corresponding nucleotide sequence in the RNA molecule; and/or
c. Three mismatches between any one of the nucleotides in position 12 to position 21 of the miRNA and the corresponding nucleotide sequence in the RNA molecule provided that there are no more than two consecutive mismatches.

As used herein, a “miRNA” is an RNA molecule of about 20 to 22 nucleotides in length which can be loaded into a RISC complex and direct the cleavage of another RNA molecule, wherein the other RNA molecule comprises a nucleotide sequence essentially complementary to the nucleotide sequence of the miRNA molecule whereby one or more of the following mismatches may occur:
d. A mismatch between the nucleotide at the 5′ end of said miRNA and the corresponding nucleotide sequence in the target RNA molecule;
e. A mismatch between any one of the nucleotides in position 1 to position 9 of said miRNA and the corresponding nucleotide sequence in the target RNA molecule;
f. Three mismatches between any one of the nucleotides in position 12 to position 21 of said miRNA and the corresponding nucleotide sequence in the target RNA molecule provided that there are no more than two consecutive mismatches; and/or
g. No mismatch is allowed at positions 10 and 11 of the miRNA (all miRNA positions are indicated starting from the 5′ end of the miRNA molecule).

A miRNA is processed from a “pre-miRNA” molecule by proteins, such as DicerLike (DCL) proteins, present in any plant cell and loaded onto a RISC complex where it can guide the cleavage of the target RNA molecules.

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop and further comprising the nucleotide sequence of the miRNA (and its complement sequence) in the double stranded RNA stem. Preferably, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

The pre-miRNA molecules (and consequently also the miRNA molecules) can be conveniently introduced into a plant cell by providing the plant cells with a gene comprising a plant-expressible promoter operably linked to a DNA region, which when transcribed yields the pre-miRNA molecule. The plant expressible promoter may be the promoter naturally associated with the pre-miRNA molecule or it may be a heterologous promoter.

### Example 1

### Methods; Generation and Confirmation of Single, Double, Triple and Quadruple ADT Knockout Lines was Accomplished

Kits.

All commercial kits were used according to the manufacturer's instructions, with any minor deviations noted.

Generation and Confirmation of Single, Double, Triple and Quadruple ADT Knockout Lines—

T-DNA insertion lines for all six ADT genes in Arabidopsis (Table 1), were obtained from either SIGnAL (34) or INRA (35). For each T-DNA insertion line, DNA was extracted from leaves of individual plants using the RedExtract kit (Sigma) with these samples then individually used as a template for two PCR reactions with different primer sets. For SALK lines, gene-specific left and right primers LP+RP, respectively, were used to amplify WT-specific PCR products, and left-border primer site “c1” (LBc1)+RP were used to amplify T-DNA-specific PCR products (Table 1: LBc1: 5′-CACAATCCCACTATCCTTCGC-3; SEQ ID NO:1). For the INRA line, the T-DNA-specific primer FLAG-LB (5′-GACGTAACATAAGGGACTGACC-3; SEQ ID NO:2) was substituted for LBc1. Homozygous T-DNA insertion lines were identified as those having T-DNA-specific PCR products only, these being sequenced to confirm the presence and the specific site of each T-DNA insertion. ADT KO lines were confirmed using RT-PCR with primers designed to the 3′-end of each ADT mRNA transcript. Confirmed single KO lines were then crossed together to generate double heterozygous ADT KOs in all combinations, with double homozygous lines being identified in the subsequent generation using the same PCR screening approach described above. The same strategy was used to create triple and quadruple KO lines, using double and triple KO parental lines, respectively. Each double, triple and quadruple KO line was independently confirmed using the PCR strategy described above.

TABLE 1
SALK and INRA T-DNA insertion lines of Arabidopsis ADTs and
corresponding primers.

| | | | Direction | | |
|---|---|---|---|---|---|
| Line | T-DNA | Insert | of T-DNA | Primer | Gene-specific primers 5′ to 3′ |
| name | insertion | position | insert | name | (SEQ ID NO:) |
| adt1 | SALK 138343 | 1^{st} Intron | 5′ to 3′ | adt1-LP | ACT GCG ATT TGT GAC ATA GGG |
| | | | | | (SEQ ID NO: 3) |
| | | | | adt1-RP | ATA TGC ACC TGG TAT CCC CTG |
| | | | | | (SEQ ID NO: 4) |
| adt2 | FLAG 353C06 | Promoter | 3′ to 5′ | adt2-LP | CAG CAG CTG ATT CGC TGT ATG C |
| | | | | | (SEQ ID NO: 5) |
| | | | | adt2-RP | TAG CGA GAG AGA GTG ATG ATG C |
| | | | | | (SEQ ID NO: 6) |
| adt3 | SALK 071907 | Exon | 3′ to 5′ | adt3-LP | CGA TCA AAC GAA ACT CCA AAG |
| | | | | | (SEQ ID NO: 7) |
| | | | | adt3-RP | AAT TCG AAG TTG CGT TTC AAG |
| | | | | | (SEQ ID NO: 8) |
| adt4 | SALK 065483 | Exon | 3′ to 5′ | adt4-LP | TCA CCA CGT GAA TAA TGA GCT C |
| | | | | | (SEQ ID NO: 9) |
| | | | | adt4-RP | TAC GTG TAG CTT ACC AAG GCG |
| | | | | | (SEQ ID NO: 10) |
| adt5 | SALK 028611 | Exon | 3′ to 5′ | adt5-LP | TAG ATT AGA TCC GTG CAT CGG |
| | | | | | (SEQ ID NO: 11) |
| | | | | adt5-RP | GCT TTT ACT GGT AGG GCT TCG |
| | | | | | (SEQ ID NO: 12) |
| adt6 | SALK 030329 | Exon | 3′ to 5′ | adt6-LP | ACC AAA TCA CTG ATA AGC CCC |
| | | | | | (SEQ ID NO: 13) |
| | | | | adt6-RP | ATA GAA CCG CCG AGA GAG TTC |
| | | | | | (SEQ ID NO: 14) |

### Example 2

### Methods; Arabidopsis Growth and Harvest Conditions were Used

Arabidopsis Growth and Harvest Conditions—

All confirmed homozygous KO and WT lines were grown in soil with four plants per pot in Washington State University greenhouses (16 h days, 27-28° C.; 8 h nights, 24-26° C.; 200 ppm nitrogen-based fertilizer added 5 days a week). For lignin analyses, the main stems of at least 48 plants were harvested weekly from after initial stem emergence up to maturity (˜3.5 to 10 weeks). The weights and lengths of 20 inflorescence stems from each line were measured, with these then subsequently cut into 0.5 to 1 cm long pieces, lyophilized and stored at room temperature prior to lignin analyses. For histochemical staining, two main stems for each ADT KO and WT line were harvested at 7 weeks.

### Example 3

### Methods; Real Time RT-PCR Analysis of ADT KO Lines was Accomplished

Real Time RT-PCR Analysis of ADT KO Lines—

Stem tissue for WT and selected ADT KO lines were harvested 5 weeks after planting, flash frozen in liquid N₂ and stored at −80° C. until use. Frozen tissue was ground using a mortar and pestle, and ˜90 to 110 mg was transferred to a 1.5 ml microcentrifuge tube. Total RNA was extracted using the Spectrum™ Plant Total RNA Extraction Kit (Sigma-Aldrich). RNA quantity and quality was assessed using a Nanodrop 2000c spectrometer (Thermo Fisher Scientific Inc.), and mRNA (1 μg) was reverse transcribed to cDNA using Superscript III (Invitrgogen). Gene-specific primers for each ADT isoform and housekeeping gene, TIP41-like (AT4G34270; GI:145352648 (Czechowski, T., et al., 2005)) were designed using Primer Premier 6.10 software (Premier Biosoft International) (see Table 2). The SYBR Green Real Time RT-PCR kit (Invitrogen) was used for real time RT-PCR reactions, with 0.05 μg cDNA and 62.5 pmol primers for each reaction. Triplicate reactions were run on a Mx 3505P Real Time Thermocycler (Stratagene), and data was analyzed with Mx Pro QPCR software (Stratagene).

TABLE 2
Primers for real time RT-PCR analysis

| | Primer sequence (5′ to 3′) |
|---|---|
| Primer Name | (SEQ ID NO:) |
| ADT1 rt-For | GTCAGATAACCGAGCAACT (SEQ ID NO: 15) |
| ADT1 rt-Rev | TACCAAAGCCACAAACCC (SEQ ID NO: 16) |
| ADT2 rt-For | TGGACACTACAATGCTCTAA (SEQ ID NO: 17) |
| ADT2 rt-Rev | TCTCAGACCTCACCTCAG (SEQ ID NO: 18) |
| ADT3 rt-For | CCGATGGATATGACTTCTTG (SEQ ID NO: 19) |
| ADT3 rt-Rev | TGACTTCACACGTTGGTT (SEQ ID NO: 20) |
| ADT4 rt-For | TGCGGAGGTTCAAGAGTA (SEQ ID NO: 21) |
| ADT4 rt-Rev | ATGCTTCTTCTGTGGATGT (SEQ ID NO: 22) |
| ADT5 rt-For | TTGGAACATCGAAGCACTT (SEQ ID NO: 23) |
| ADT5 rt-Rev | AGGAATGACGTGTACTCTTG (SEQ ID NO: 24) |
| ADT6 rt-For | CGAGGTTCAGGAGTTTACA (SEQ ID NO: 25) |
| ADT6 rt-Rev | TGGTTACGATGAAGTTGATG (SEQ ID NO: 26) |
| TIP41 like | GTTCCTCCTCTTGCGATT (SEQ ID NO: 27) |
| rt-For | |
| TIP41-like | CAGTTGGTGCCTCATCTT (SEQ ID NO: 28) |
| rt-Rev | |

### Example 4

### Methods; Arogenate Dehydratase Activity Assays and Free Amino Acid Analysis

Arogenate Dehydratase Activity Assays and Free Amino Acid Analysis—

The following method from Jung et al., 1986 (2), modified by Maeda et al., 2010 (15), was applied for assaying ADT activity in Arabidopsis stems. Approximately 20 g of stem tissue was harvested, immediately ground in liquid N₂, and extracted with 30 ml lysis buffer (20 mM Tris-HCl, pH 8.0, 1 mM ethylenediaminetetraacetic acid, 1 mM dithiothreitol, 10% glycerol, 1 mM phenylmethylsulfonyl fluoride, 35 mg leupeptin, and 35 mL of plant cell and tissue extract protease inhibitor cocktail [Sigma-Aldrich, #p9599]). The crude lysate was then subjected to an ammonium sulfate precipitation, with both the 20-40% and 40-80% fractions collected. Each fraction was desalted with a PD-10 column (GE Healthcare) then concentrated to approximately 500 μL using an Amicon Ultra-4 Centrifugal Filter (Millipore). A 5 μl aliquot of the protein extract (containing 30 mg and 370 mg for the 20 to 40% and 40 to 80% fractions, respectively) were added to the total volume of the 12 μL reaction mixture containing 250 μM arogenate and 20 mM Tris, pH 8.0. After incubation at 37° C. for 15 min, the reaction was stopped by addition of 10 μl of MeOH with 2 μl of 10 mM alanine added as internal standard. The assay mixtures were vortexed and centrifuged, with half of the sample derivatized with N-methyl-N-(tert-butyldimethylsilyl) trifluoroacetamide and analyzed by GC-MS, and the other half being derivatized using the Pico-tag system (Waters), and analyzed by HPLC as previously described (1, 5). No enzymatic phenylalanine formation was detected under the conditions employed.

Total free amino acid pools were extracted from 5 week old WT and ADT KO stems, using methanol:chloroform:water (12:5:3), as previously described (Corea, O. R. A., et al, 2011). Amino acids were derivatized using the AccQ•Tag™ Ultra Derivatization Kit (Waters) and analyzed by Ultra-Performance Liquid Chromatography (Waters). Phe and Tyr levels in WT were ˜18 and ˜12 pmol/mg dry weight, respectively, while those of the ADT KO lines ranged from 12-21 and 5-11, pmol/mg dry weight, respectively, suggesting that no obvious changes between WT and ADT KO lines.

### Example 5

### Methods; Histochemical Staining

Histochemical Staining—

Histochemical staining and imaging of fresh hand-cut sections taken near the base of Stage 3 (Altamura et al., 2001) (7 weeks old) stems of both mutants and WT lines were carried out as previously described by Patten et al, 2010 (17). For detection of presumed G-lignin, stem cross-sections were placed in a phloroglucinol-HCl (0.1%, w/v) solution for 30 min (38, 39), transferred to a glass slide and observed under the differential interference contrast setting, using an Olympus System Microscope, Model BHT (Olympus Optical Co., Ltd, Tokyo, Japan). The Made reaction was used for presumed S-lignin component detection; hand cut cross-sections of stems were treated for 10 min with KMnO₄ (0.5%, w/v, filtered through a 0.45 nm MillexHV filter) and rinsed with distilled H₂O (40). Samples were then treated with HCl (0.1%, v/v) for 5 min, rinsed, mounted in concentrated NH₄OH, with observation as described above.

### Example 6

### Methods; Laser Microscope Dissection of Vascular Bundles and Interfascicular Regions

Laser Microscope Dissection of Vascular Bundles and Interfascicular Regions—

Laser microscope dissection was used to separate the vascular bundles (vb) and interfascicular fiber (if) regions from 7 week old stems at Stage 3 (8 week) for WT and adt1/4/5 lines, as described previously (17). Briefly, sections were made using a Cryocut 1800 microtome (Leica Microsystems), and laser microdissected using a P.A.L.M.s Microbeam System (P.A.L.M. Microlaser Technologies) to isolate the vb and if regions. Approximately 5000 samples from each region were individually collected for adt1/4/5 and WT, respectively. Each dissected sample was combined, then individually transferred to glass vials and extracted twice for 12 h with H₂O:acetone (3:7, v:v) at RT, and dried in vacuo in preparation for pyrolysis GC/MS analyses.

### Example 7

### Methods; Estimations of Lignin Contents and Compositions

Estimations of Lignin Contents and Compositions—

Extractive-free stem cell wall residues (CWR) were obtained through extraction with EtOH:toluene (1:1, v/v), EtOH and distilled H₂O, as described previously (17, 18, 41, 42). Estimations of lignin contents were made using the AcBr method (43), as described in Blee et al, 2001 (44) and adjusted for G:S monomeric rations, using estimated AcBr extinction coefficients (280 nm) of 15.31, 18.61 and 14.61 g⁻¹ cm⁻¹ for H, G and S units, respectively (18). Cleavable monomeric (G/S) compositions and contents were also estimated by thioacidolysis using the general procedures of Lapierre et al., 1986 (45) and Rolando et al., 1992 (46), as described by Blee et al., 2001 (44), and quantified by comparison using standard curves with authentic standards. Pyrolysis GC/MS was carried out as previously described (17) using a pyrolyzer PYR-4A:Shimadzu interfaced with a Shimadzu GC-17A gas chromatograph and a Shimadzu QP-5000 mass spectrometer (Shimadzu Scientific Instruments INC, Columbia, Md., USA), with the following modifications. Vapor phase products were separated on a SPB-5 poly (5% diphenyl 95% dimethyl siloxane) nonpolar column (30 m×0.25 μm) (Sigma-Aldrich Corp. St. Louis, Mo., USA) using a 5° C. min⁻¹ gradient beginning from 50 to 270° C. Pyrolysis products were identified by comparison to the following authentic standards: phenol (1), 4-methylphenol (2), 3-methylphenol (3), guaiacol (4), 4-ethylphenol (5), 4-methylguaiacol (6), catechol (7), 4-vinylphenol (8), 4-allylphenol (9), 4-ethylguaiacol (10), 4-vinylguaiacol (11), trans-propenylphenol (12), eugenol (14), vanillin (15), trans-isoeugenol (17), acetovanillone (18), guaiacylacetone (19), 4-allyl-2,6, dimethoxyphenol (21), syringaldehyde (22), trans-coniferyl alcohol (24), acetosyringone (25), trans-sinapyl aldehyde (27), trans-coniferyl aldehyde (29), as well as to the literature mass spectroscopic fragmentation data of 2,6 dimethoxyphenol (13), 2,6 dimethoxy 4-methylphenol (16), 2,6 dimethoxy 4-vinylphenol (20), trans-2,6 dimethoxy 4-propenylphenol (23) and syringylacetone (26) (17, 47, 48).

### Example 8

### Results; ADT Knockout Lines were Obtained; Generation and Gross Phenotype Comparisons of ADT Knockout Lines

Generation and Gross Phenotype Comparisons of ADT Knockout Lines—

T-DNA insertion lines for each ADT isoenzymes used in this study (Table 1) were obtained from either the SALK Institute Genomic Analysis Laboratory (SIGnAL) (34), or the Institut National de la Recherche Agronomique (INRA) (35). Following screening to obtain the various homozygous lines, it was determined that ADT1 and ADT3-6 had their corresponding mRNA transcripts abolished (FIG. 1); these lines were thus considered as confirmed single KO lines. The exception was ADT2 (the sole line obtained from INRA) in which ADT2 mRNA was still present in the homozygous T-DNA insertion line, and this was not utilized further. The five confirmed single KO lines: adt1, adt3, adt4, adt5 and adt6 were then crossed together to generate double ADT KO lines in all combinations: adt1/3, adt1/4, adt1/5, adt1/6, adt3/4, adt3/5, adt3/6, adt4/5, adt4/6, and adt5/6, respectively. Similarly, confirmed homozygous double KOs were crossed together to create triple KOs, with adt1/3/4, adt1/3/5, adt1/4/5, adt3/4/5 and adt4/5/6 also being obtained. Additionally, an adt3/4/5/6 quadruple knockout was generated by crossing together adt3/4/5 and adt4/5/6.

The Arabidopsis growth/development time-frame involves three distinct stages (49), as previously described in the comprehensive study of lignin pathway altered ref8, fah 1-2 and C4H::F5H Arabidopsis lines (17). In the current study, all of the plant lines also went through each of these growth/development stages to reach maturity (stage 3) by 7 weeks, and then until senescence (10 weeks). During this time-frame, phenotypic assessment of the above ADT mutant lines at each weekly harvesting point was carried out, in order to identify single or double KO lines with potential reductions in lignin contents, alterations in stem lengths and weights, and/or presence of prostrate phenotypes. Of the single KOs, only adt5 had a slight decrease in stem weights and lengths (to ˜90% of WT levels, FIGS. 3A and 3B), and had a partially prostrate phenotype that was phenotypically distinct from WT (FIG. 4E). The remaining single KO lines, adt1, adt3 and adt4 each had relatively similar stem lengths and weights to WT over the course of growth and development (FIGS. 3A and 3B), and also did not appear visually to be phenotypically different from WT (FIGS. 4B-4D). Additionally, one double KO, adt4/5, initially stood out as having reduced stem weights and lengths to ˜82% of WT levels (FIGS. 3A and 3B), and a rather prostrate phenotype (FIG. 4F) relative to WT.

Based on these observations, additional crosses were made to obtain two triple KOs that shared the same combination of adt4 and adt5; i.e. adt1/4/5 and adt3/4/5. Both triple KOs also had obvious prostrate phenotypes (FIGS. 4G and 414 and adt1/4/5 had similar stem weights and lengths to adt4/5, corresponding to ˜85% of WT levels whereas adt3/4/5 displayed further reductions in both stem weight and length, corresponding to ˜71% of WT levels (FIGS. 32, A and 3B). Interestingly, the ADT isoenzymes ADT3, ADT4 and ADT5, which are in the same phylogenetic cluster, ‘subgroup III’ (3), appeared to cause the greatest phenotypic effect, and therefore it was instructive to test if the fourth ADT in that subgroup (ADT6) caused any further changes. Thus, Applicants also obtained a quadruple KO, adt3/4/5/6, in which all members of subgroup III (3) were disrupted. This line had further reductions in stem weights and lengths (FIGS. 3A and 3B) corresponding to ˜67% of WT levels, as well as displaying a prostrate phenotype (FIG. 41).

Based on these phenotype analyses, the single KOs adt1, adt3, adt4 and adt5, double KO adt4/5, triple KOs adt1/4/5 and adt3/4/5, and quadruple KO adt3/4/5/6 were subjected to both histochemical and lignin analyses in order to assess the individual and/or combinatorial contribution of these genes to lignification. Lignin analyses (estimated lignin contents and monomeric compositions) were carried out weekly (see below).

### Example 9

### Results; ADT Expression Levels in Selected ADT Knockout lines

ADT Expression Levels in Selected ADT Knockout Lines—

Real-time RT-PCR was used to verify that ADT transcript(s) were absent in each corresponding ADT KO line, and additionally, to identify any potential increased expression in non-targeted ADTs. The relative mRNA expression levels of all six ADTs were measured in WT and each of the eight selected KO lines described above. For each of these lines, ADT transcripts for each corresponding knocked out gene were absent, except for ADT1 and ADT6, which were reduced to <25% WT levels (FIGS. 10A-10F). It is possible that the latter two genes are able to produce a small amount of transcript, despite being homozygous for T-DNA insertions, since the insertions in ADT1 and ADT6 are present in the 1^{st} intron and the 5′UTR, respectively, and may allow for a small amount of correctly-processed mRNA to be transcribed from these genes. Similar findings have been described elsewhere for T-DNA insertion mutants (36).

Consistent with the findings by Rippert et al. (13), ADT4 and ADT5 had the highest expression levels in WT stems, with the remaining ADTs also being expressed in stem, but at lower levels (FIGS. 10A-10F). Expression of these two genes was increased slightly, but significantly, compared to WT in certain lines: ADT4 was increased in the adt3 and adt5 KO lines, while ADT5 was increased in the adt3 KO, while no change in expression was observed in the add and adt4 KO lines. Small increases in expression were also noted for ADT1, ADT2 and ADT3, for certain ADT KO lines (FIGS. 10A-10F), while there were no apparent increases observed for ADT6 in any lines.

As a decrease in expression levels does not (necessarily) translate directly to a decrease in overall ADT activity, we also attempted to measure ADT activity in Arabidopsis stems. While ADT activity has been detected in certain species (2, 3) it has never been described in Arabidopsis. With this in mind, crude enzyme extracts were performed using WT stem tissue, using the modified protocol described by Maeda et al. (15). Both arogenate and prephenate were tested as substrates, however, in both cases no ADT or PDT activity could be observed (data not shown). Since no activity could be detected in WT stem, the KO lines were not assayed since they were expected to have even lower levels of ADT activity. Furthermore, levels of Phe and Tyr in stem tissue were measured at weekly time points for WT and ADT KO lines, however, significant changes in Phe and/or Tyr levels were not observed in these samples (data not shown).

### Example 10

### Results; Histochemical Analyses of Single and Multiple ADT Knockouts

Histochemical Analyses of Single and Multiple ADT Knockouts—

Qualitative histochemical analyses of 7 week old basal stem sections (FIG. 5A-5O, and FIGS. 9A-9H), for both staining of guaiacyl (G) and syringyl (S) lignin containing phenotypes, were also carried out using phloroglucinol-HCl (for G) (17, 38, 39) and Mäule (for S) (17, 40) reagents, respectively. G-lignin component staining, used frequently for detection of coniferyl-alcohol derived moieties (see FIG. 6 (bottom structures), was near identical throughout the vb and if regions of WT, adt1, adt3 and adt4 lines (FIGS. 9A-9D) lines, whereas presumed G moieties were apparently less readily detectable in if regions of adt5, adt4/5, adt1/4/5, adt3/4/5 and adt3/4/5/6 (FIGS. 5B-5F), as gauged by the decreased levels of red-pink staining in these cross-sections. Also apparent in the phloroglucinol-HCl treated sections were irregularly-shaped and partially collapsed cell walls in the metaxylem (mx) within the vb of adt1/4/5 and adt3/4/5 lines (FIGS. 5H and 5I), in contrast to the WT line where this deformation was not evident (FIG. 5G). There was little, if any, visible difference in presumed S (sinapyl alcohol derived) moieties for any of the KO lines using the Mäule reagent (FIGS. 5K-5O and FIGS. 9F-9H), as compared to WT (FIG. 5J and FIG. 9E).

### Example 11

### Results; Estimated Lignin Contents/Compositions of Single and Multiple ADT Knockouts

Estimated Lignin Contents/Compositions of Single and Multiple ADT Knockouts—

The potential effects on lignification in the various ADT KO lines were next assessed, relative to the WT line, with stems from each plant line harvested weekly. Extractive-free stem cell wall residues (CWR) were thus subjected to “acetyl bromide (AcBr) lignin” and thioacidolysis (17, 18, 46) analyses, in order to estimate gross lignin amounts and cleavable lignin monomeric contents/compositions. Note though that “AcBr lignin” contents were provisionally corrected for their H:G:S compositions, using extinction co-efficients for individual G and S lignin-enriched preparations, as described elsewhere (18). The thioacidolysis treatment, by contrast, releases monomeric H—, G- and S-derived constituents, via cleavage of presumed 8-O-4′ interunit linkages in the lignin biopolymer(s), to afford the corresponding monomeric thioethylated derivatives, respectively (FIGS. 6A-6D).

### Example 12

### Results; Analysis of “AcBr Lignin” Deposition Patterns

Analysis of “AcBr lignin” Deposition Patterns—

Over the growth and development of the Arabidopsis lines being studied, the estimated “AcBr lignin” contents in the WT line plateaued at ˜23.6% cell wall residue (CWR) at full maturation (i.e. average of 7 to 10 weeks) (FIG. 6A; Table 3). The adt KO lines identified in our phenotype comparisons were also analyzed, and their estimated lignin contents were compared to WT. The adt1 and adt3 lines reached comparable levels to WT of ˜23.8 and ˜23.0% CWR, respectively, whereas the adt4 line putatively had a slightly higher lignin level of ˜26.0% CWR. By contrast, the adt5 line had a marked reduction in estimated “AcBr lignin” content to ˜18.9% CWR at maturity, whereas an even more striking reduction (i.e. to ˜14.4% CWR) was noted with the double mutant adt4/5. Further decreases in “AcBr lignin” levels were documented with the triple mutants; at maturity, adt1/4/5 and adt3/4/5 had roughly equivalent “AcBr lignin” levels of ˜12.2 and 11.9% CWR, respectively, while adt3/4/5/6 was reduced to ˜7.5% CWR. (Note though that the “AcBr lignin” methods can over-estimate lignin amounts due to presence of non-lignin UV-absorbing components (see Anterola and Lewis (10)).

TABLE 3
Estimated lignin contents for WT and adt knockout lines

| | | G | | S | | G + S | | AcBr | |
|---|---|---|---|---|---|---|---|---|---|
| | Weeks | μmol/g± | % WT | μmol/g± | % WT | μmol/g ± S.D. | % WT | % CWR | % WT |
| WT | 3.5 | 68.5 ± 0.8 | | 5.9 ± 0.4 | | 74.4 ± 1.1 | | 11.6 | |
| | 4 | 121.2 ± 10.6 | | 33.1 ± 5.9 | | 154.3 ± 15.8 | | 16.8 | |
| | 5 | 191.6 ± 3.9 | | 58.9 ± 3.2 | | 250.5 ± 1.4 | | 22.1 | |
| | 6 | 199.5 ± 14.1 | | 65.9 ± 4.7 | | 265.4 ± 18.4 | | 22.4 | |
| | 7 | 246.2 ± 2.9 | | 63.5 ± 0.4 | | 309.7 ± 3.3 | | 22.1 | |
| | 8 | 231.9 ± 2.8 | | 73.4 ± 0.8 | | 305.3 ± 3.5 | | 24.1 | |
| | 9 | 217.9 ± 6.8 | | 76.2 ± 8.0 | | 294.1 ± 14.8 | | 23.7 | |
| | 10 | 214.7 ± 14.1 | | 72.0 ± 3.8 | | 286.7 ± 17.9 | | 24.7 | |
| | Avg. 7-10 | 227.7 | | 71.3 | | 299.0 | | 23.6 | |
| adt1 | 3.5 | 23.4 ± 0.1 | 34 | 1.4 ± 0.1 | 24 | 24.8 ± 012 | 33 | 9.4 | 81 |
| | 4 | 144.7 ± 7.0 | 119 | 26.3 ± 1.2 | 79 | 171.0 ± 8.2 | 111 | 16.5 | 98 |
| | 5 | 138.2 ± 1.3 | 72 | 35.7 ± 1.7 | 61 | 173.9 ± 3.0 | 69 | 18.7 | 84 |
| | 6 | 181.6 ± 2.7 | 91 | 46.6 ± 2.2 | 71 | 228.2 ± 4.9 | 86 | 22.6 | 101 |
| | 7 | 209.1 ± 0.1 | 85 | 54.6 ± 0.1 | 86 | 263.7 ± 0.1 | 85 | 24.3 | 110 |
| | 8 | 196.2 ± 14.8 | 85 | 55.5 ± 4.5 | 76 | 251.7 ± 19.2 | 82 | 24.0 | 100 |
| | 9 | 210.1 ± 17.4 | 96 | 57.4 ± 9.3 | 75 | 267.5 ± 26.7 | 91 | 22.6 | 96 |
| | 10 | 193.7 ± 13.1 | 90 | 52.3 ± 4.4 | 73 | 246.0 ± 17.5 | 86 | 24.0 | 97 |
| | Avg. 7-10 | 202.3 | 89 | 55.0 | 77 | 257.2 | 86 | 23.8 | 101 |
| adt3 | 3.5 | 34.8 ± 7.5 | 51 | 1.7 ± 1.9 | 29 | 36.5 ± 9.4 | 49 | 6.0 | 52 |
| | 4 | 139.8 ± 13.5 | 115 | 30.9 ± 3.0 | 93 | 170.7 ± 16.5 | 111 | 15.2 | 91 |
| | 5 | 108.3 ± 0.1 | 57 | 25.3 ± 0.1 | 43 | 133.6 ± 0.1 | 53 | 16.6 | 75 |
| | 6 | 192.1 ± 2.9 | 96 | 49.3 ± 3.6 | 75 | 241.4 ± 6.6 | 91 | 20.9 | 93 |
| | 7 | 190.0 ± 0.1 | 77 | 42.4 ± 0.1 | 67 | 232.4 ± 0.1 | 75 | 23.7 | 107 |
| | 8 | 175.2 ± 9.6 | 76 | 50.6 ± 4.2 | 69 | 225.8 ± 13.7 | 74 | 24.1 | 100 |
| | 9 | 196.5 ± 8.0 | 90 | 54.3 ± 2.7 | 71 | 250.8 ± 10.7 | 85 | 21.3 | 90 |
| | 10 | 213.4 ± 15.1 | 99 | 53.1 ± 4.9 | 74 | 266.5 ± 20.0 | 93 | 22.7 | 92 |
| | Avg. 7-10 | 193.8 | 85 | 50.1 | 70 | 243.9 | 82 | 23.0 | 97 |
| adt4 | 3.5 | 60.2 ± 1.5 | 88 | 8.2 ± 2.5 | 139 | 68.4 ± 2.4 | 92 | 10.0 | 86 |
| | 4 | 120.2 ± 11.0 | 99 | 19.5 ± 1.6 | 59 | 139.7 ± 12.6 | 91 | 16.3 | 97 |
| | 5 | 196.7 ± 16.4 | 103 | 53.6 ± 7.9 | 91 | 250.3 ± 23.1 | 100 | 20.6 | 93 |
| | 6 | 252.2 ± 27.2 | 126 | 67.5 ± 9.5 | 102 | 319.7 ± 36.4 | 120 | 25.6 | 114 |
| | 7 | 233.2 ± 2.2 | 95 | 58.3 ± 2.0 | 92 | 291.5 ± 3.7 | 94 | 26.1 | 118 |
| | 8 | 279.5 ± 2.9 | 121 | 77.1 ± 1.7 | 105 | 356.6 ± 1.1 | 117 | 25.0 | 104 |
| | 9 | 236.6 ± 16.3 | 109 | 79.1 ± 5.4 | 104 | 315.7 ± 21.7 | 107 | 26.4 | 112 |
| | 10 | 237.5 ± 21.7 | 111 | 73.9 ± 5.8 | 103 | 311.4 ± 27.5 | 109 | 26.4 | 107 |
| | Avg. 7-10 | 246.7 | 109 | 72.1 | 101 | 318.8 | 107 | 26.0 | 110 |
| adt5 | 3.5 | 25.8 ± 1.9 | 38 | n.d.^{a} | 0 | 25.8 ± 1.9 | 35 | 7.5 | 64 |
| | 4 | 88.3 ± 6.2 | 73 | 27.2 ± 7.0 | 82 | 115.5 ± 13.2 | 75 | 12.2 | 73 |
| | 5 | 107.1 ± 2.0 | 56 | 50.1 ± 0.3 | 85 | 157.2 ± 2.2 | 63 | 16.8 | 76 |
| | 6 | 128.4 ± 15.4 | 64 | 61.9 ± 6.5 | 94 | 190.3 ± 21.9 | 72 | 18.7 | 83 |
| | 7 | 166.7 ± 0.8 | 68 | 77.4 ± 1.4 | 122 | 244.1 ± 2.2 | 79 | 16.8 | 76 |
| | 8 | 150.8 ± 20.8 | 65 | 80.0 ± 2.3 | 109 | 230.8 ± 6.7 | 76 | 20.1 | 83 |
| | 9 | 169.7 ± 9.2 | 78 | 83.3 ± 4.7 | 109 | 253.0 ± 13.8 | 86 | 18.2 | 77 |
| | 10 | 179.3 ± 3.4 | 84 | 91.0 ± 2.1 | 126 | 270.3 ± 5.5 | 94 | 20.3 | 82 |
| | Avg. 7-10 | 166.6 | 74 | 82.9 | 117 | 249.5 | 84 | 18.9 | 80 |
| adt4/5 | 3.5 | 23.6 ± 4.5 | 34 | n.d.^{a} | 0 | 23.6 ± 4.5 | 33 | 7.8 | 67 |
| | 4 | 56.5 ± 5.9 | 47 | 23.8 ± 1.4 | 72 | 80.3 ± 6.3 | 52 | 11.5 | 69 |
| | 5 | 55.2 ± 10.3 | 29 | 49.0 ± 7.8 | 83 | 104.2 ± 18.0 | 42 | 11.4 | 51 |
| | 6 | 70.0 ± 2.2 | 35 | 67.6 ± 2.6 | 103 | 137.6 ± 3.4 | 52 | 13.5 | 60 |
| | 7 | 71.4 ± 2.7 | 29 | 66.8 ± 3.6 | 105 | 138.2 ± 6.3 | 45 | 13.6 | 62 |
| | 8 | 69.7 ± 7.2 | 30 | 60.1 ± 0.4 | 82 | 129.7 ± 7.5 | 43 | 14.0 | 58 |
| | 9 | 81.8 ± 3.0 | 38 | 66.8 ± 2.4 | 88 | 148.6 ± 4.7 | 51 | 14.3 | 60 |
| | 10 | 94.6 ± 1.5 | 44 | 78.1 ± 1.4 | 108 | 172.7 ± 2.9 | 60 | 15.7 | 63 |
| | Avg. 7-10 | 79.4 | 35 | 68.0 | 96 | 147.3 | 49 | 14.4 | 61 |
| adt1/4/5 | 3.5 | 20.1 ± 2.1 | 29 | 3.7 ± 0.7 | 63 | 23.8 ± 2.8 | 32 | 7.2 | 62 |
| | 4 | 25.4 ± 0.5 | 21 | 21.6 ± 0.1 | 65 | 47.0 ± 0.6 | 30 | 9.9 | 59 |
| | 5 | 61.2 ± 1.8 | 32 | 43.8 ± 1.1 | 74 | 105.0 ± 3.0 | 42 | 10.3 | 46 |
| | 6 | 73.4 ± 1.2 | 37 | 65.3 ± 4.8 | 99 | 138.7 ± 5.4 | 52 | 11.1 | 50 |
| | 7 | 71.1 ± 1.7 | 29 | 60.1 ± 1.9 | 95 | 131.2 ± 3.6 | 42 | 9.7 | 44 |
| | 8 | 66.4 ± 7.2 | 29 | 53.5 ± 1.3 | 73 | 119.9 ± 3.3 | 39 | 12.1 | 50 |
| | 9 | 66.0 ± 3.7 | 30 | 43.2 ± 4.0 | 57 | 109.2 ± 3.3 | 37 | 13.1 | 55 |
| | 10 | 59.4 ± 0.2 | 28 | 45.4 ± 0.7 | 63 | 104.8 ± 1.4 | 37 | 14.2 | 57 |
| | Avg. 7-10 | 65.7 | 29 | 50.6 | 72 | 116.3 | 39 | 12.2 | 52 |
| adt3/4/5 | 3.5 | 12.4 ± 3.2 | 18 | 2.1 ± 0.8 | 36 | 14.5 ± 4.0 | 19 | 7.6 | 65 |
| | 4 | 16.6 ± 1.6 | 14 | 13.4 ± 2.0 | 40 | 30.0 ± 3.3 | 19 | 8.2 | 49 |
| | 5 | 43.0 ± 0.8 | 22 | 39.3 ± 4.0 | 67 | 82.3 ± 4.8 | 33 | 8.4 | 38 |
| | 6 | 36.8 ± 4.3 | 18 | 40.5 ± 8.1 | 61 | 77.3 ± 12.4 | 29 | 11.6 | 47 |
| | 7 | 42.5 ± 0.3 | 17 | 37.6 ± 0.8 | 59 | 80.1 ± 1.1 | 26 | 10.4 | 47 |
| | 8 | 54.9 ± 1.8 | 24 | 53.0 ± 2.5 | 72 | 107.9 ± 4.3 | 35 | 10.9 | 45 |
| | 9 | 43.2 ± 4.9 | 20 | 43.7 ± 8.4 | 57 | 86.9 ± 3.4 | 30 | 11.9 | 50 |
| | 10 | 43.7 ± 1.8 | 20 | 32.5 ± 1.2 | 45 | 76.2 ± 0.3 | 27 | 14.2 | 57 |
| | Avg. 7-10 | 46.1 | 20 | 41.7 | 58 | 87.8 | 29 | 11.9 | 50 |
| adt3/4/5/6 | 3.5 | 21.2 ± 1.5 | 31 | 5.3 ± 0.4 | 90 | 26.5 ± 1.9 | 36 | 5.0 | 43 |
| | 4 | 23.8 ± 4.3 | 20 | 15.1 ± 3.4 | 46 | 38.9 ± 7.7 | 25 | 5.7 | 34 |
| | 5 | 42.2 ± 0.1 | 22 | 46.1 ± 1.1 | 78 | 88.3 ± 1.2 | 35 | 6.3 | 28 |
| | 6 | 48.4 ± 2.9 | 24 | 52.5 ± 6.0 | 80 | 100.9 ± 8.9 | 38 | 8.1 | 36 |
| | 7 | 45.4 ± 8.1 | 18 | 48.1 ± 4.1 | 76 | 93.5 ± 12.2 | 30 | 6.6 | 30 |
| | 8 | 50.3 ± 2.7 | 22 | 48.8 ± 1.8 | 66 | 99.1 ± 4.3 | 32 | 7.2 | 30 |
| | 9 | 46.7 ± 9.9 | 21 | 45.2 ± 9.2 | 59 | 91.9 ± 3.4 | 31 | 8.3 | 35 |
| | 10 | 48.4 ± 2.1 | 23 | 48.5 ± 4.2 | 67 | 96.9 ± 0.3 | 34 | 7.9 | 32 |
| | Avg. 7-10 | 47.7 | 21 | 47.7 | 67 | 95.4 | 32 | 7.5 | 32 |

^{a}not detected

### Example 13

### Results; Thioacidolysis Analyses and Lignin Monomer-Derived Compositions and Contents

Thioacidolysis Analyses and Lignin Monomer-Derived Compositions and Contents—

The thioacidolysis analysis results for the CWRs for the WT and KO lines are depicted in FIGS. 6B-6D (with actual values given in Table 3 above). In agreement with the estimated AcBr lignin analyses, thioacidolysis showed somewhat similar trends as regards the amounts of G+S monomeric moieties released from their lignins from the beginning of stem elongation until full maturity (average of 7 to 10 weeks). In this regard, cleavage of the presumed 8-O-4′ interunit linkages in WT released ˜299 mmol G+S monomers/g CWR at maturation (average of 7 to 10 weeks), while adt4 was slightly higher at ˜319 mmol/g CWR. These values correspond to circa 25% of the estimated AcBr lignin content. The other single KOs adt1, adt3 and adt5 were, however, slightly reduced compared to WT, with ˜257, 244 and 250 mmol/g CWR of G+S monomers released, respectively. Larger reductions were also observed for the double KO, adt4/5 (−147 mmol/g CWR), triple KOs, adt1/4/5 and adt3/4/5 (−116 and ˜88 mmol/g CWR, respectively) and quadruple KO, adt3/4/5/6 (95 mmol/g CWR). Their amounts ranged from ˜27-16% of the putative lignin present, with such reductions in releasable monomers being frequently observed when overall lignin contents are reduced.

Thioacidolysis data also showed differential effects on releasable monomeric G+S, G, and S moieties (FIGS. 6B-6D, respectively). Relative to WT, adt4 apparently had a very slight increase in both releasable monomeric G and S moieties, while adt1 and adt3 had a slight decrease in both (FIG. 6B). By contrast, the adt5 line was significantly decreased in G (FIG. 6C), but had slightly higher levels of S monomers compared to WT (FIG. 6D). Similarly, the double adt4/5 KO had even more pronounced reductions in G monomer levels released, with little effect on the S amounts compared to WT. However, in the corresponding triple and quadruple KOs, adt1/4/5, adt3/4/5 and adt3/4/5/6, there were further reductions in both G and S releasable monomers, compared to the adt4/5 KO.

### Example 14

### Results; Pyrolysis GC/MS Analyses

Pyrolysis GC/MS Analyses—

It was next instructive to compare the lignin-derived pyrolysis products released from S-enriched if and G-enriched vb tissues using both WT and adt1/4/5 lines, in a manner as previously successfully carried out on ref8, fah 1-2 and C4H::F5H lines (Patten et al., 2010). First, WT and adt1/4/5 stem CWR samples were individually subjected to pyrolysis GC/MS and analyzed. Products were identified by either retention time, co-elution with authentic standards, and mass spectroscopic fragmentation data. Simple inspection of releasable lignin-derived pyrolysis products in these two GC/MS chromatograms indicated substantial reductions in G and S components (relative to H-derived moieties) in the adt1/4/5 line (FIG. 7B) on comparison to WT (FIG. 7A), with the largest reduction being with G-derived components. That is, in the WT line, the pyrolysis products identified were of H (peaks 1-3, 5 and 8), G (peaks 4, 6, 7, 10, 11, 14, 15, 17, 19, 24 and 29) and S (peaks 13, 20 and 23), respectively, with the G-components most abundant (FIG. 7A and Table 4). By contrast, the pyrolysis products obtained for the adt1/4/5 cross-sections detected the presence of H— (peaks 1-3, and 5), G (peaks 4, 6, 10, 11, 14, 15 and 19) and S (peaks 13, 20, 23 and 26) with significant reduction in both G/S derived products relative to H-moieties (FIG. 7B and Table 4). These data thus agreed with thioacidolysis results that also indicated a greater reduction in G-lignin-derived monomers as compared to S-monomeric moieties in the adt1/4/5 line.

TABLE 4
Pyrolysis GC/MS lignin-derived products from Arabidopsis WT and
adt1/4/5 cell wall residues (CWR), as well as laser microdissected vascular
bundles (vb) and interfascicular fibers (if).

| | Compound | Unit | Ions |
|---|---|---|---|
| 1 | Phenol | H | 94, 66, 65 |
| 2 | 4-Methylphenol | H | 108, 107, 90, 79, 77, 51 |
| 3 | 3-Methylphenol | H | 108, 107, 90, 79, 77, 51 |
| 4 | Guaiacol | G | 124, 109, 81, 53 |
| 5 | 4-Ethylphenol | H | 122, 107, 77, 51 |
| 6 | 4-Methylguaiacol | G | 138, 123, 95, 77, 67 |
| 7 | Catechol | G | 110, 92, 81, 64, 63 |
| 8 | 4-Vinylphenol | H | 120, 119, 91, 65 |
| 9 | 4-Allylphenol^{a} | H | 134, 133, 107, 105, 91, 77 |
| 10 | 4-Ethylguaiacol | G | 152, 137, 122 |
| 11 | 4-Vinylguaiacol | G | 150, 135, 107, 77 |
| 12 | trans-4-propenylphenol | H | 134, 133, 105, 77 |
| 13 | 2,6-Dimethoxyphenol | S | 154, 139, 111, 96, 65 |
| 14 | Eugenol | G | 164, 149, 137, 131, 103, |
| | | | 91, 77 |
| 15 | Vanillin | G | 152, 151, 123, 109, 81 |
| 16 | 2-6,Dimethoxy-4-methylphenol | S | 168, 153, 125, 65 |
| 17 | trans-isoeugenol | G | 164, 149, 131, 103, 91, 77 |
| 18 | Acetovanillone^{a} | G | 166, 151, 123 |
| 19 | Guaiacylacetone | G | 180, 137, 122 |
| 20 | 2,6-Dimethoxy-4-vinylphenol | S | 180, 165, 137, 122 |
| 21 | 4-Allyl-2,6-dimethoxyphenol | S | 194, 179, 147, 131, 119, |
| | | | 91, 77 |
| 22 | Syringaldehyde^{a} | S | 182, 167, 153, 139, 111, 93 |
| 23 | trans-2,6-dimethoxy-4- | S | 194, 179, 151, 131 |
| | propenylphenol | | |
| 24 | trans-coniferyl alcohol | G | 180, 137, 124 |
| 25 | Acetosyringone^{a} | S | 196, 181, 153 |
| 26 | Syringylacetone | S | 210, 167, 123, 69 |
| 27 | trans-sinapyl aldehyde^{a} | S | 208, 180, 177, 165, 137 |
| 28 | trans-sinapyl alcohol^{a} | S | 210, 167, 154, 149 |
| 29 | Coniferyl aldehyde | G | 178, 161, 147, 135 |

Next, laser micro-dissection was employed to excise circa 5000 individual vb and if sections from the WT and adt1/4/5 lines, with these also being subjected to pyrolysis GC/MS. Analysis of the vb regions of adt1/4/5 (FIG. 7D and Table 4) resulted in identification of H-(peaks 1-3 and 5), G (peaks 4, 6, 10, 11, 15, 17 and 19) and S (peaks 13, 20 and 23) pyrolysis fragments, with both G and S moieties being significantly reduced relative to the H-derived components (with the S-moieties being barely detectable). By contrast, the analysis of the if regions gave a chromatogram with H— (peaks 1-3 and 5), G- (peaks 4, 11 and 17) and S-peaks 13, 20 and 21) pyrolysis fragments (FIG. 7C and Table 4). In this case, however, the relative amounts of S-components still remained high, with the most notable effect being on G-component reductions. These results are thus consistent with histochemical staining using phloroglucinol, which indicated that the reduction was greatest in the G-lignin constituents of the if.

### Example 15

### Down-Regulation of Arogenate Dehydratases in Poplar was Achieved Using RNAi

Methods.

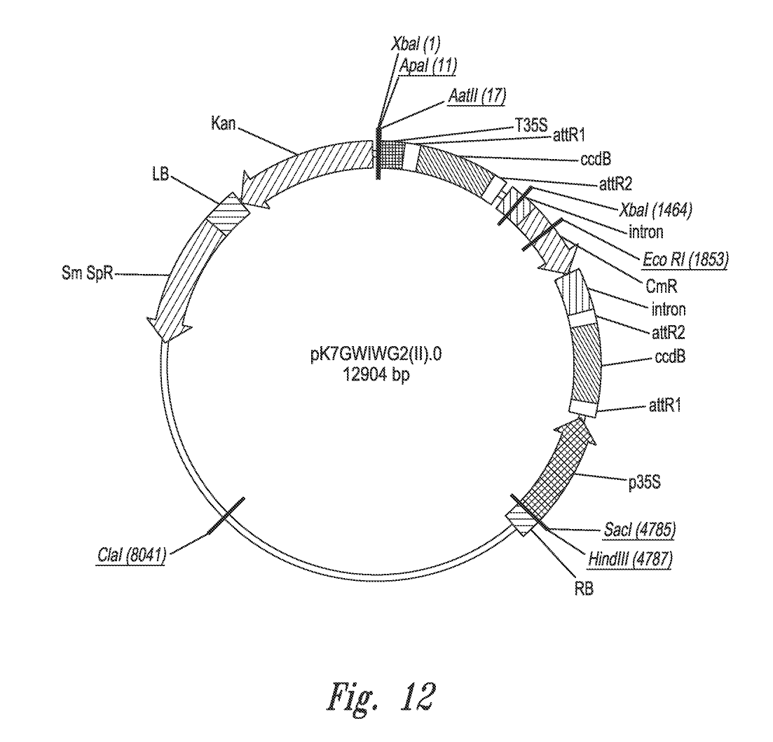

Based on the Populus trichocarpa database, ADT1 (PtADT1), ADT2 (PtADT2) and ADT3 (PtADT3) genes were individually isolated by PCR amplification. After full cDNA sequence verification, ˜500 bp of coding regions from the start codon were individually re-amplified for use in RNAi vector construction. The PtADT1, PtADT2, PtADT1::2 (fused) and PtADT3-RNAi regions (SEQ ID NOS:94-96, respectively, for PtADT1, PtADT2 and PtADT3-RNAi regions) were individually cloned into pK7GWIWG2(II) RNAi vector (FIG. 12) (Karimi et al., 2002). Agrobacterium tumefaciens EHA105 strain was individually transformed with the RNAi vector constructs (PtADT1-RNAi, PtADT2-RNAi, PtADT3-RNAi and PtADT1::2-RNAi) using the freeze/thaw shock transformation method (An et al., 1988).

The transformed Agrobacterium was cultured overnight in YEP medium (5 ml) containing suitable antibiotics at 28° C. with shaking. The cells were pelleted by centrifugation at 3,000×g for 10 min and then resuspended in 1M medium (Table 6) to achieve an OD_{600 nm} of 0.1-0.3.

TABLE 6
Medium for poplar transformation

| IM medium | 4.4 | g/L | MS Salt (with vitamins) |
|---|---|---|---|
| | 10 | mM | Galactose |
| | 1.28 | mM | MES |
| | 50 | μM | Acetosyringone |
| Wash solution | 4.4 | g/L | MS Salt (with vitamins) |
| | 1.0 | μM | NAA |
| | 1.0 | μM | BAP |
| | 1.0 | μM | 2iP |
| | 250 | mg/ml | Ascorbic acid |
| | 200 | mg/ml | Timentin |
| CIM1 medium | 4.4 | g/L | MS Salt (without vitamins) |
| | 30 | g/L | Sucrose |
| | 0.25 | g/L | MES |
| | 0.1 | g/L | myo-Inositol |
| | 10 | μM | NAA |
| | 5 | μM | 2iP |
| | 5 | g/L | Agar, TC |
| | 200 | mg/L | L-glutamine |
| | 1x | | FV vitamins* |
| CIM2 medium | 4.4 | g/L | MS Salt (without vitamins) |
| | 30 | g/L | Sucrose |
| | 0.25 | g/L | MES |
| | 0.1 | g/L | myo-Inositol |
| | 10 | μM | NAA |
| | 5 | μM | 2iP |
| | 5 | g/L | Agar, TC |
| | 200 | mg/L | L-glutamine |
| | 1x | | FV vitamins* |
| | 50 | mg/ml | Kanamycin |
| | 200 | mg/ml | Timentin |
| SIM medium | 4.4 | g/L | MS Salt (without vitamins) |
| | 30 | g/L | Sucrose |
| | 0.25 | g/L | MES |
| | 0.1 | g/L | myo-Inositol |
| | 5 | g/L | Agar, TC |
| | 0.2 | μM | Thidiazuron, TDZ |
| | 200 | mg/L | L-glutamine |
| | 1x | | FV vitamins* |
| | 100 | mg/ml | Kanamycin |
| | 200 | mg/ml | Timentin |
| SEM medium | 4.4 | g/L | MS Salt (without vitamins) |
| | 30 | g/L | Sucrose |
| | 0.25 | g/L | MES |
| | 0.1 | g/L | myo-Inositol |
| | 5 | g/L | Agar, TC |
| | 0.1 | μM | BAP |
| | 200 | mg/L | L-glutamine |
| | 1x | | FV vitamins* |
| | 100 | mg/ml | Kanamycin |
| | 200 | mg/ml | Timentin |
| RIM medium | 2.2 | g/L | MS Salt (without vitamins) |
| | 20 | g/L | Sucrose |
| | 0.25 | g/L | MES |
| | 0.1 | g/L | myo-Inositol |
| | 5 | g/L | Agar, TC |
| | 0.5 | μM | IBA |
| | 200 | mg/L | L-glutamine |
| | 1x | | FV vitamins* |
| | 25 | mg/ml | Kanamycin |
| | 100 | mg/ml | Timentin |

*FV vitamins (100 ×)—0.05 g Nicotinic acid, 0.05 g Pyridoxine HCl, 0.05 g L-Cycteine, 0.05 g Calcium pantothenate, 0.05 g Thiamine HCl, 1 mg/L Biotin, dissolve in 500 ml D.W.

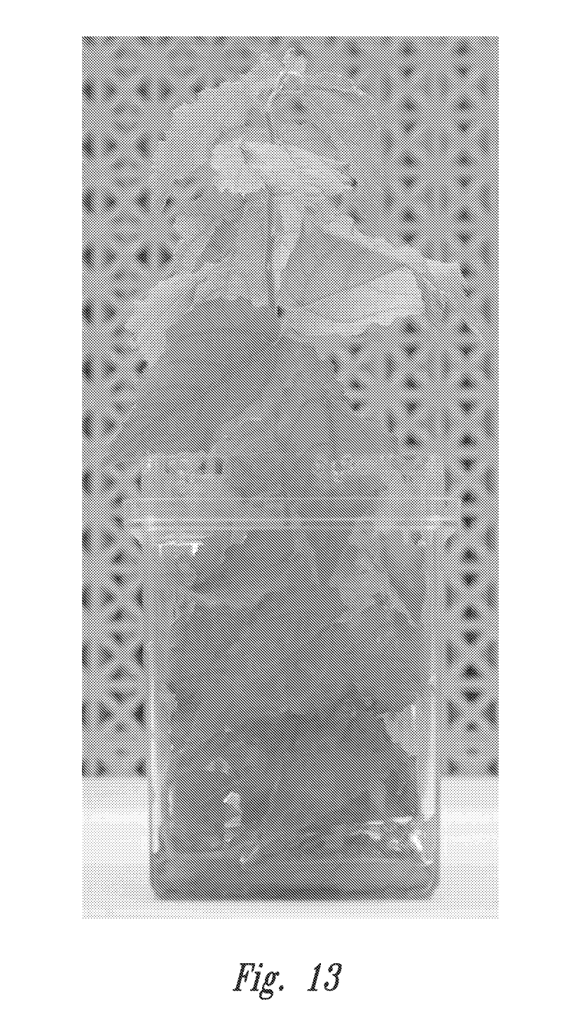

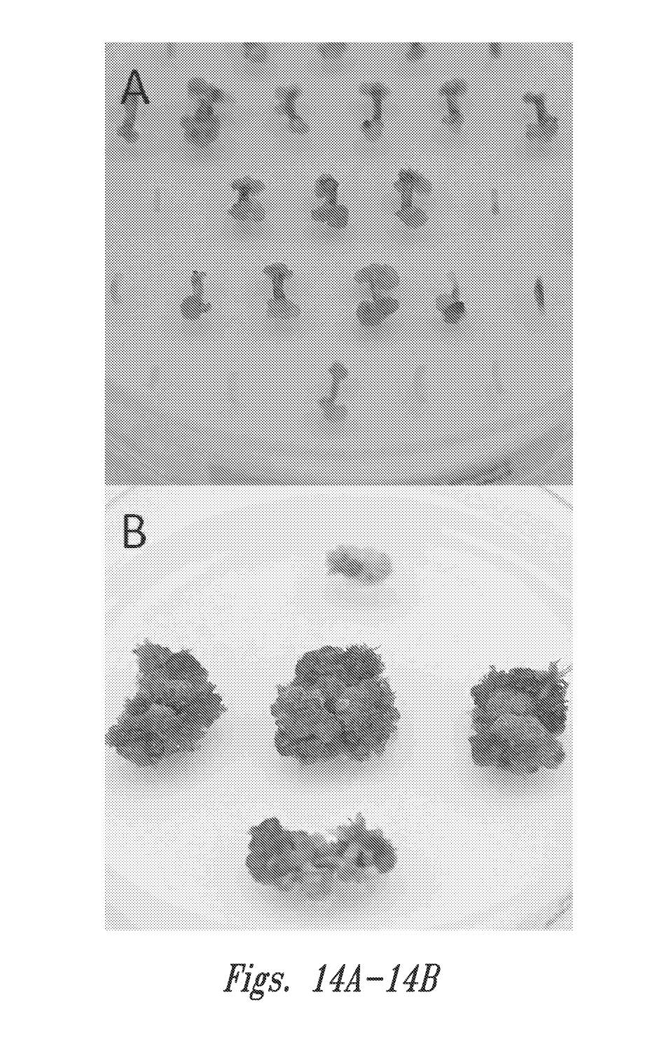

Forty- to fifty-day-old, in vitro grown hybrid poplar (Populus tremula×P. alba, INRA 717-1B4, female) (FIG. 13) was utilized for transformation (Filichkin et al., 2006). Leaves, petioles and stems were cut in 3-4 mm sections and incubated with the Agrobacterium suspension for 1 hour under slow agitation. After removing the excess Agrobacterium suspension, the plant segments were placed on a Callus Induction Medium 1 (CIM1, Table 6) plate which was next sealed with parafilm. Co-cultivation on CIM1 medium was carried out for 3 days at 22° C. in the dark. The co-cultivated explants were next carefully washed with sterile water (30 ml, 4 times) to remove most of the Agrobacterium, rinsed with a Wash solution (Table 6; 30 ml) and finally placed on a Callus Induction Medium 2 (CIM2, Table 6) plate which was sealed with parafilm. The CIM2 plates were then incubated for 21 days at 22° C. in the dark (FIG. 14A).

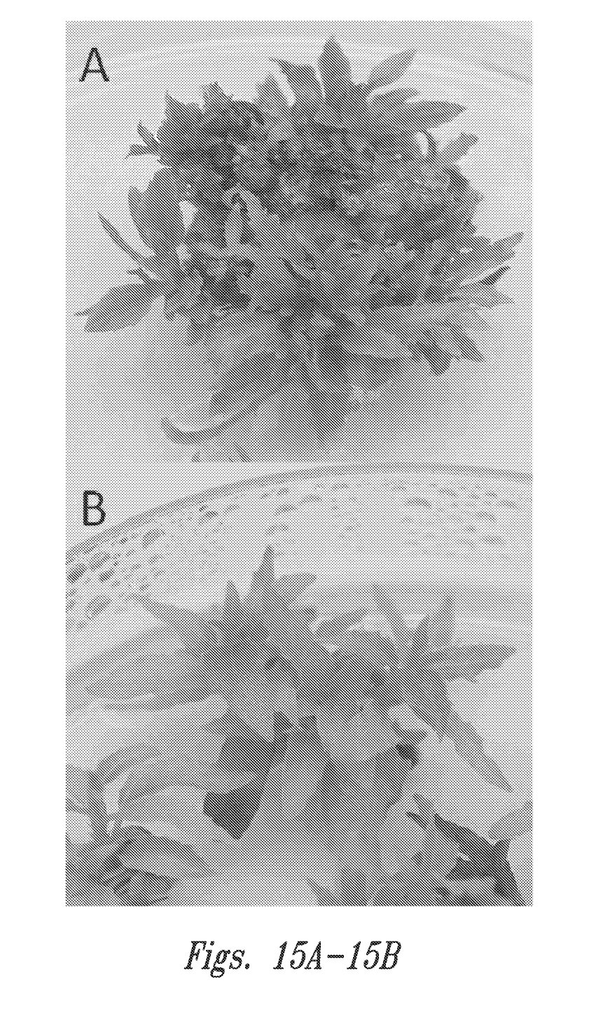

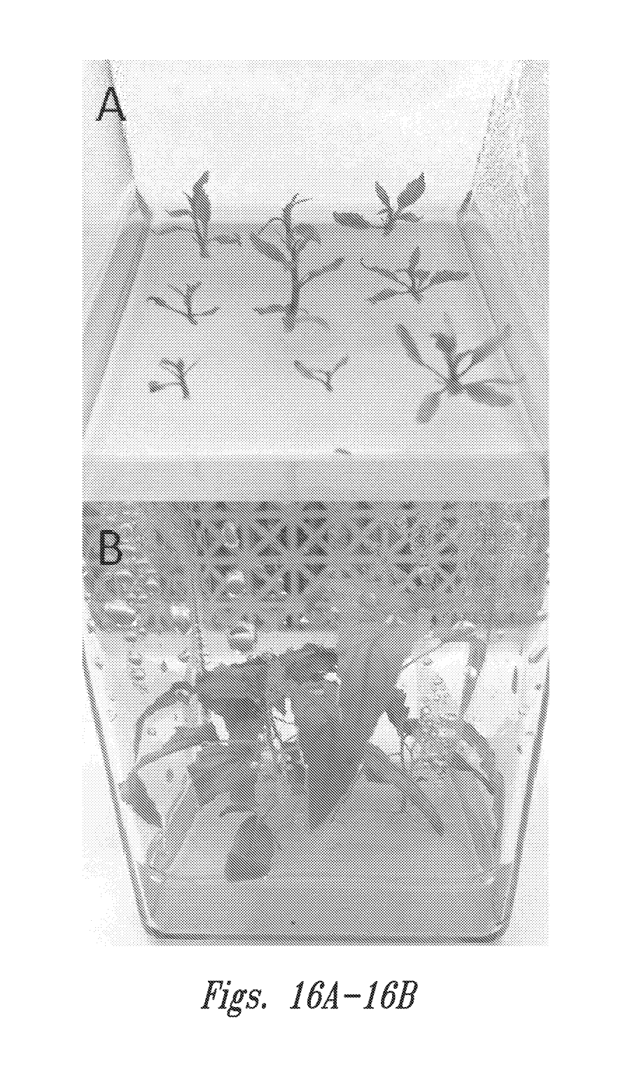

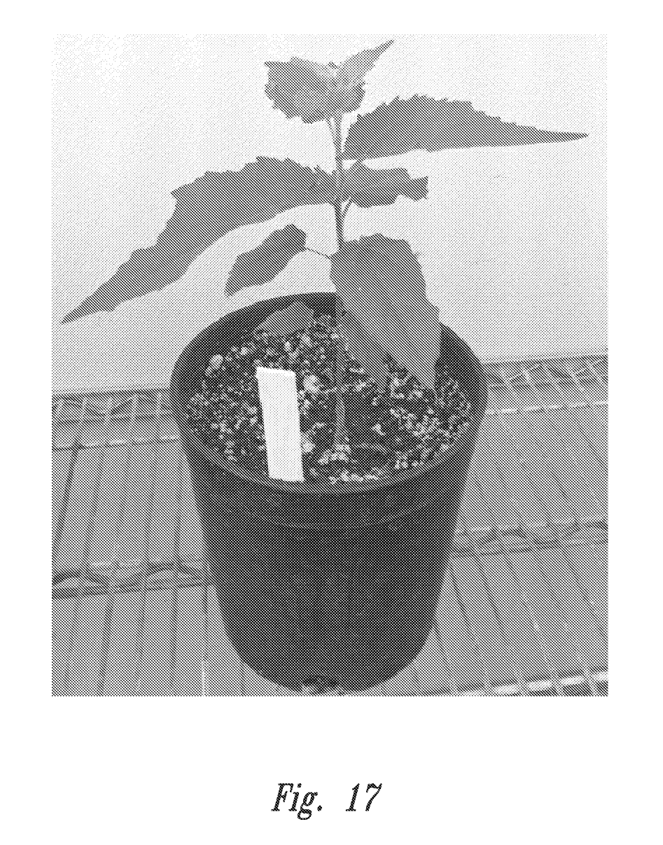

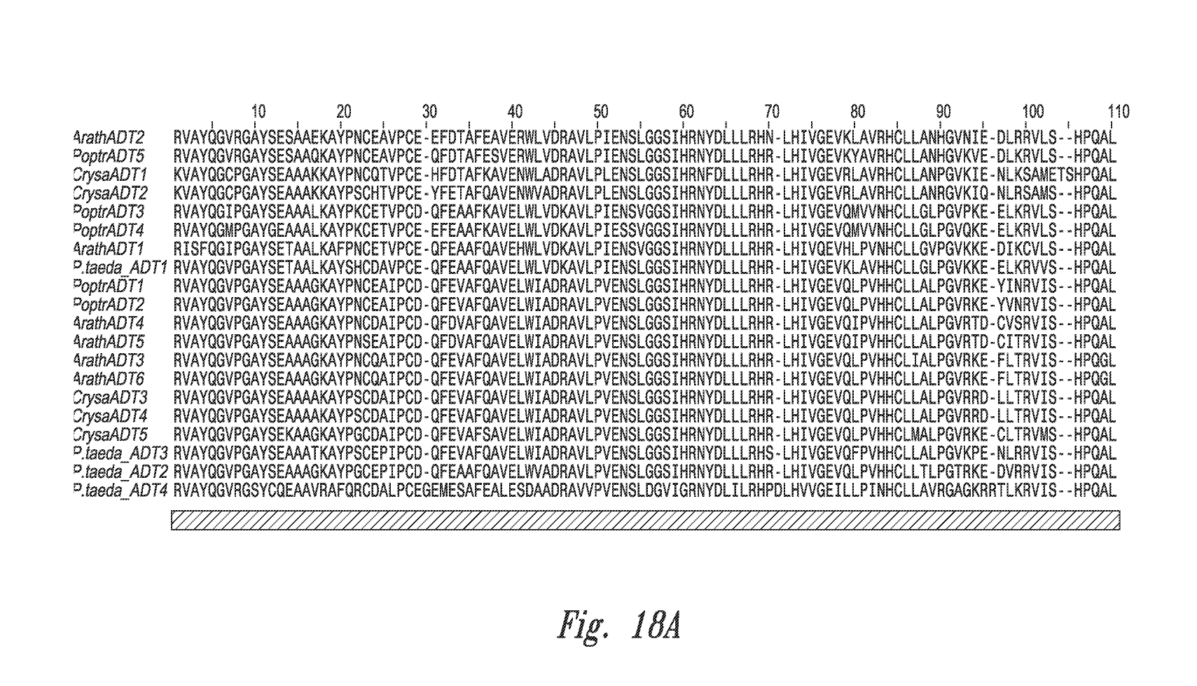

Calli from the CIM2 plates were next transferred to Shoot Induction Medium (SIM, Table 1) plates, with the latter kept at 22° C. under light (FIG. 3B). After 21 to 42 days, adventitious shoot clumps with visible leaflets (FIG. 15A) were subcultured on a Stem Elongation Medium (SEM, Table 6) plate for shoot elongation. After 21 days on the SEM medium (FIG. 15B), the elongated shoots were transferred onto Root Induction Medium (RIM, Table 6) in a Magenta box to induce roots (FIG. 16A). Finally, thirty to forty days grown transgenic hybrid poplar (FIG. 16B) were transferred to soil and then grown in growth chamber (FIG. 17).

The RNA express ion levels were individually determined by using real-time qPCR amplification.

According to particular aspects, down-regulation of arogenate dehydratases in poplar was achieved using RNAi.

### Example 16

### Amino Acid Sequences of ADTs from Arabidopsis, Pinus taeda, Populus trichocarpa and Oryza sativa

According to further aspects, reducing or eliminating ADT enzyme expression is broadly applicable across all plants (e.g., vascular plants) (e.g., hardwood, softwood, graminae and angiosperms, etc.), for example, in methods of reducing lignin by reducing the expression of one or more chloroplast ADT genes [e.g., that comprise the TRF motif (between positions 170-180 in the alignment of FIG. 16; which is the ADT active site), or other motifs (LTKIESRP motif; positions 228-235 in the alignment of FIG. 16) that are conserved for chloroplast expressed genes], via using knockouts or via RNAi techniques.

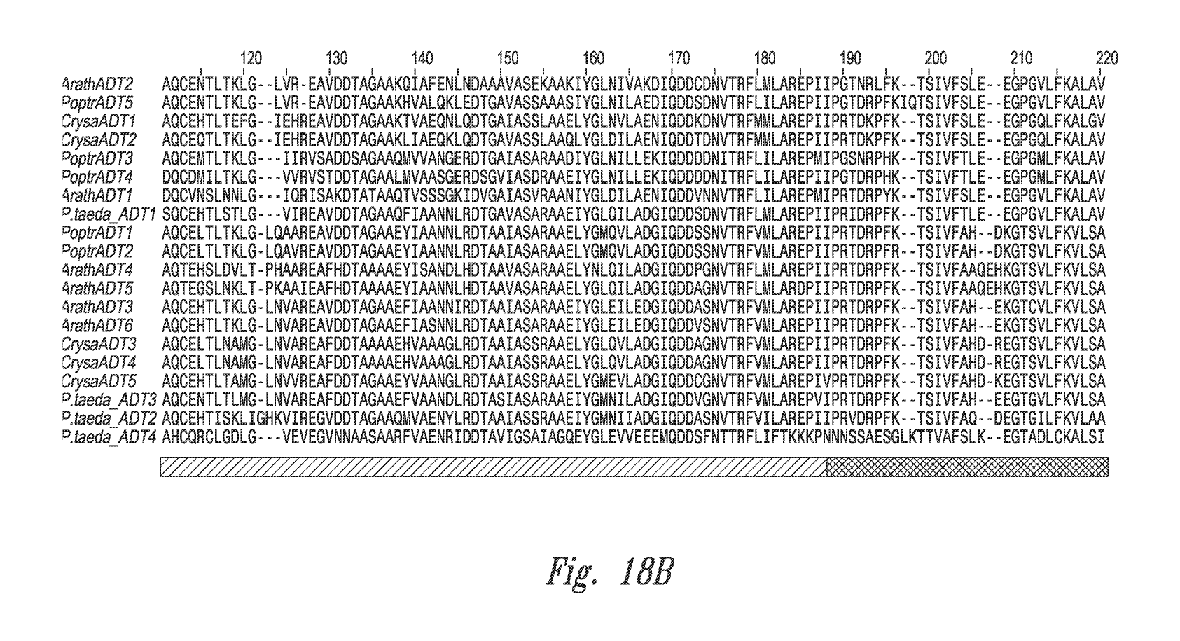

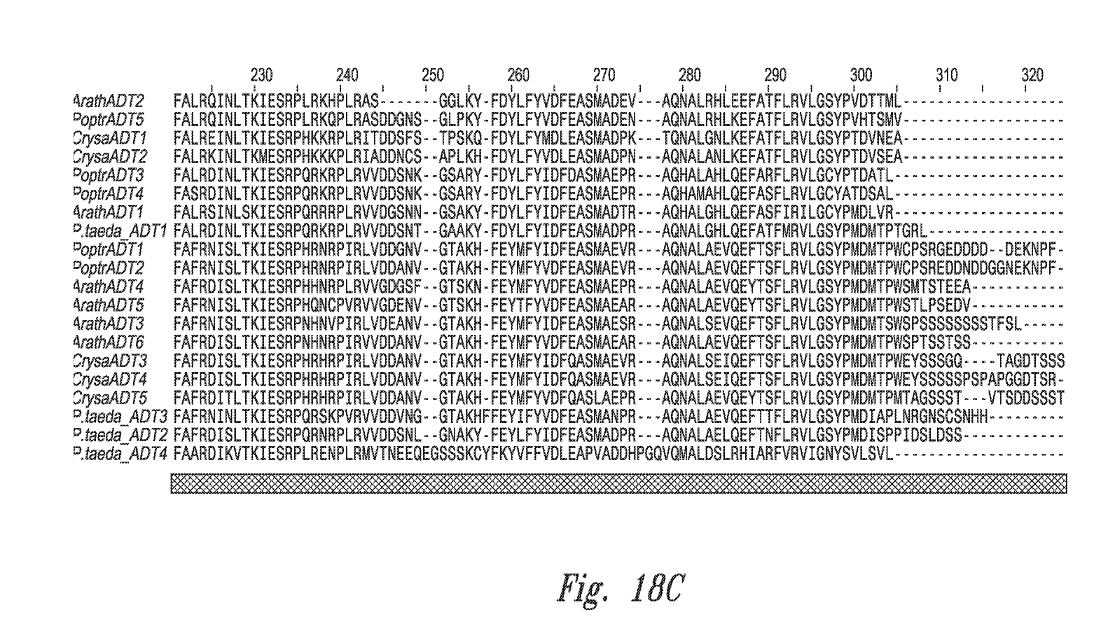

FIG. 18 shows an alignment of ADTs from Arabidopsis, Pine, Poplar, Rice, showing the ADT domain in green; ACT domain in red; and sequences conserved in >50% of samples are shaded in blue, where darker shades represent greater conservation. The alignment of ADT genes of Arabidopsis, pine, poplar and rice that shows conserved motifs (particularly the TRF motif located between alignment positions 170-180 that is the ADT active site).

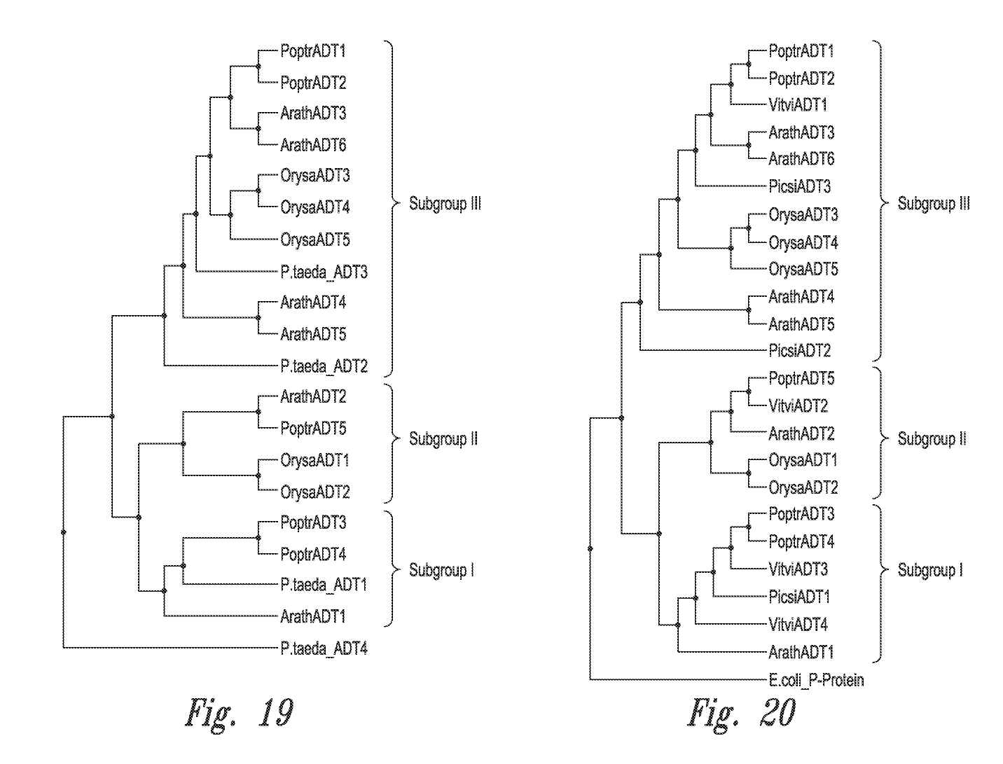

FIG. 19 shows phylogenetic clustering of ADTs.

Exemplary Sequences Used, with Corresponding Accession Numbers Shown
Arabidopsis thaliana (Nomenclature from Ehlting et A, 2005; see Table 7 below for accession numbers)

| | |
|---|---|
| ArathADT1 = At1g11790 | (SEQ ID NOS: 29-32) |
| ArathADT2 = At3g07630 | (SEQ ID NOS: 33-36) |
| ArathADT3 = At2g27820 | (SEQ ID NOS: 37-38) |
| ArathADT4 = At3g44720 | (SEQ ID NOS: 39-40) |
| ArathADT5 = At5g22630 | (SEQ ID NOS: 41-42) |
| ArathADT6 = At1g08250 | (SEQ ID NOS: 43-44) |

Oryza sativa (Nomenclature based on comparison to Arabidopsis ADTs)

OrysaADT1 = Os03t0286200

| | | |
|---|---|---|
| NM_001056307.1 | GI: 115452342 | (SEQ ID NOS: 45 and 46) |

OrysaADT2 = Os07t0694600 [ADT Identified by Yamada et al (2008)]

| | | |
|---|---|---|
| NM_001067275.1 | GI: 115474282 | (SEQ ID NOS: 47 and 48) |

OrysaADT3 = Os09t0566050

| | | |
|---|---|---|
| NM_001070487.1 | GI: 115480716 | (SEQ ID NOS: 92 and 93) |

OrysaADT4 = Os09t0565700

| | | |
|---|---|---|
| NM_001070485.1 | GI: 115480712 | (SEQ ID NOS: 49 and 50) |

OrysaADT5 = Os04t0406600

| | | |
|---|---|---|
| NM_001059251.1 | GI: 115458231 | (SEQ ID NOS: 51 and 52) |

“ADT-like genes” (Not included in Alignment):
OrysaADT6 = Os10t0523700 *No ESRP Motif

| | | |
|---|---|---|
| NM_001071638.1 | GI: 115483019 | (SEQ ID NOS: 53 and 54) |

OrysaADT7 = Os07t0512000 *No TRF or ESRP Motif*
(*Not included in alignment due to absence of TRF Motif (ADT active site))

Populus trichocarpa (Nomenclature from Tsai et al. (54); incorporated by reference herein in its entirety, and particularly for sequence information)

| PoptrADT1 = | XM_002305530.1 | GI: 224078591 | (SEQ ID NOS: 55 |
|---|---|---|---|
| 594833 | | | and 56) |
| PoptrADT2 = | XM_002328037.1 | GI: 224131643 | (SEQ ID NOS: 57 |
| 721464 | | | and 58) |
| PoptrADT3 = | XM_002316499.1 | GI: 224113660 | (SEQ ID NOS: 59 |
| 568031 | | | and 60) |
| PoptrADT4 = | XM_002313615.1 | GI: 224105004 | (SEQ ID NOS: 61 |
| 817706 | | | and 62) |
| PoptrADT5 = | XM_002312677.1 | GI: 224102528 | (SEQ ID NOS: 63 |
| 565256 | | | and 64) |

Pinus taeda (Loblolly pine) (Nomenclature arbitrary)
P.taedaADT1=TA10224 3352
P.taedaADT2=TA10516 335
P.taedaADT3=TC59573
P.taedaADT4=TC57322
Picea sitchensis (Sitka spruce); exemplary sequences

| | | | |
|---|---|---|---|
| PicsiADT1 | ABR17815.1 | GI: 148909434 | (SEQ ID NOS: 65 and 66) |

(based on identity with P. taedaADT1)

| | | | |
|---|---|---|---|
| PicsiADT2 | ACN39903.1 | GI: 224284335 | (SEQ ID NOS: 67 and 68) |

(based on identity with P. taedaADT2)

| | | | |
|---|---|---|---|
| PicsiADT3 | EF676789.1 | GI: 148907054 | (SEQ ID NOS: 97 and 98) |

Vitis vinefera (Nomenclature arbitrary)

| | | |
|---|---|---|
| VitviADT1 | XP_002268701.1, GI: 225436339 | |
| XM_002268665.1 | GI: 225436338 | (SEQ ID NOS: |
| | | 99 and 100) |
| VitviADT2 | XP_002282017.1, GI: 225435199 | |
| XM_002281981.1 | GI: 225435198 | (SEQ ID NOS: |
| | | 101 and 102) |
| VitviADT3 | XP_002268124.1, GI: 225444487 | |
| XM_002268088.1 | GI: 225444486 | (SEQ ID NOS: |
| | | 103 and 104) |
| VitviADT4 | XP_002269463.1, GI: 225447035 | |
| XM_002269427.1 | GI: 225447034 | (SEQ ID NOS: |
| | | 105 and 106) |

TABLE 7
Exemplary Arabidopsis thaliana ADT sequences

| ADT Gene | mRNA accession no. | Protein accession no. |
|---|---|---|
| ADT 1 | NM_001035947.1 | NP_001031024.1 |
| | GI: 79317656 | GI: 79317657 |
| | (SEQ ID NO: 29) | (SEQ ID NO: 30) |
| ADT 1 | NM_101051.2 | NP_172644.1 |
| | GI: 30682412 | GI: 15221096 |
| | (SEQ ID NO: 31) | (SEQ ID NO: 32) |
| ADT 2 | NM_111642.2 | NP_187420.1 |
| | GI: 30680385 | GI: 15231489 |
| | (SEQ ID NO: 33) | (SEQ ID NO: 34) |
| ADT 2 | NM_202520.2 | NP_974249.1 |
| | GI: 145362426 | GI: 42572307 |
| | (SEQ ID NO: 35) | (SEQ ID NO: 36) |
| PD1 | NM_128342.2 | NP_180350.1 |
| (ADT 3) | GI: 30683658 | GI: 15226259 |
| | (SEQ ID NO: 37) | (SEQ ID NO: 38) |
| ADT 4 | NM_114340.3 | NP_190058.1 |
| | GI: 30692262 | GI: 15230489 |
| | (SEQ ID NO: 39) | (SEQ ID NO: 40) |
| ADT 5 | NM_122169.2 | NP_197655.1 |
| | GI: 30688569 | GI: 15242928 |
| | (SEQ ID NO: 41) | (SEQ ID NO: 42) |
| ADT 6 | NM_100698.3 | NP_563809.1 |
| | GI: 42561798 | GI: 18390869 |
| | (SEQ ID NO: 43) | (SEQ ID NO: 44) |

Protein sequences used in alignment (showing conserved ‘ADT’ and ‘ACT’ domains only) (E. coli PDT sequence included for an outgroup):

ArathADT 1
(SEQ ID NO: 69)
RISFQGIPGAYSETAALKAFPNCETVPCEQFEAAFQAVEHWLVDKAVLPIENSVGGSI
HRNYDLLLRHRLHIVQEVHLPVNHCLLGVPGVKKEDIKCVLSHPQALDQCVNSLNN
LGIQRISAKDTATAAQTVSSSGKIDVGAIASVRAANIYGLDILAENIQDDVNNVTRFLI
LAREPMIPRTDRPYKTSIVFSLEEGPGVLFKALAVFALRSINLSKIESRPQRRRPLRVV
DGSNNGSAKYFDYLFYIDFEASMADTRAQHALGHLQEFASFIRILGCYPMDLVR
ArathADT2
(SEQ ID NO: 70)
RVAYQGVRGAYSESAAEKAYPNCEAVPCEEFDTAFEAVERWLVDRAVLPIENSLGG
SIHRNYDLLLRHNLHIVGEVKLAVRHCLLANHGVNIEDLRRVLSHPQALAQCENTLT
KLGLVREAVDDTAGAAKQIAFENLNDAAAVASEKAAKIYGLNIVAKDIQDDCDNVT
RFLMLAREPIIPGTNRLFKTSIVFSLEEGPGVLFKALAVFALRQINLTKIESRPLRKHPL
RASGGLKYFDYLFYVDFEASMADEVAQNALRHLEEFATFLRVLGSYPVDTTML
ArathADT3
(SEQ ID NO: 71)
RVAYQGVPGAYSEAAAGKAYPNCQAIPCDQFEVAFQAVELWIADRAVLPVENSLGG
SIHRNYDLLLRHRLHIVGEVQLPVHHCLIALPGVRKEFLTRVISHPQGLAQCEHTLTK
LGLNVAREAVDDTAGAAEFIAANNIRDTAAIASARAAEIYGLEILEDGIQDDASNVTR
FVMLAREPIIPRTDRPFKTSIVFAHEKGTCVLFKVLSAFAFRNISLTKIESRPNHNVPIR
LVDEANVGTAKHFEYMFYIDFEASMAESRAQNALSEVQEFTSFLRVLGSYPMDMTS
WSPSSSSSSSSTFSL
ArathADT4
(SEQ ID NO: 72)
RVAYQGVPGAYSEAAAGKAYPNCDAIPCDQFDVAFQAVELWIADRAVLPVENSLG
GSIHRNYDLLLRHRLHIVGEVQIPVHHCLLALPGVRTDCVSRVISHPQALAQTEHSLD
VLTPHAAREAFHDTAAAAEYISANDLHDTAAVASARAAELYNLQILADGIQDDPGN
VTRFLMLAREPIIPRTDRPFKTSIVFAAQEHKGTSVLFKVLSAFAFRDISLTKIESRPHH
NRPLRVVGDGSFGTSKNFEYMFYVDFEASMAEPRAQNALAEVQEYTSFLRVLGSYP
MDMTPWSMTSTEEA
ArathADT5
(SEQ ID NO: 73)
RVAYQGVPGAYSEAAAGKAYPNSEAIPCDQFDVAFQAVELWIADRAVLPVENSLGG
SIHRNYDLLLRHRLHIVGEVQIPVHHCLLALPGVRTDCITRVISHPQALAQTEGSLNKL
TPKAAIEAFHDTAAAAEYIAANNLHDTAAVASARAAELYGLQILADGIQDDAGNVT
RFLMLARDPIIPRTDRPFKTSIVFAAQEHKGTSVLFKVLSAFAFRNISLTKIESRPHQNC
PVRVVGDENVGTSKHFEYTFYVDFEASMAEARAQNALAEVQEYTSFLRVLGSYPMD
MTPWSTLPSEDV
ArathADT6
(SEQ ID NO: 74)
RVAYQGVPGAYSEAAAGKAYPNCQAIPCDQFEVAFQAVELWIADRAVLPVENSLGG
SIHRNYDLLLRHRLHIVGEVQLPVHHCLLALPGVRKEFLTRVISHPQGLAQCEHTLTK
LGLNVAREAVDDTAGAAEFIASNNLRDTAAIASARAAEIYGLEILEDGIQDDVSNVTR
FVMLAREPIIPRTDRPFKTSIVFAHEKGTSVLFKVLSAFAFRDISLTKIESRPNHNRPIRV
VDDANVGTAKHFEYMFYVDFEASMAEARAQNALAEVQEFTSFLRVLGSYPMDMTP
WSPTSSTSS
OrysaADT1
(SEQ ID NO: 75)
KVAYQGCPGAYSEAAAKKAYPNCQTVPCEHFDTAFKAVENWLADRAVLPLENSLG
GSIHRNFDLLLRHRLHIVGEVRLAVRHCLLANPGVKIENLKSAMetSHPQALAQCEHT
LTEFGIEHREAVDDTAGAAKTVAEQNLQDTGAIASSLAAELYGLNVLAENIQDDKD
NVTRFMMLAREPIIPRTDKPFKTSIVFSLEEGPGQLFKALGVFALREINLTKIESRPHKK
RPLRITDDSFSTPSKQFDYLFYMDLEASMADPKTQNALGNLKEFATFLRVLGSYPTD
VNEA
OrysaADT2
(SEQ ID NO: 76)
KVAYQGCPGAYSEAAAKKAYPSCHTVPCEYFETAFQAVENWVADRAVLPLENSLG
GSIHRNYDLLLRHRLHIVGEVRLAVRHCLLANRGVKIQNLRSAMSHPQALAQCEQTL
TKLGIEHREAVDDTAGAAKLIAEQKLQDTGAVASSLAAQLYGLDILAENIQDDTDNV
TRFMMLAREPIIPRTDKPFKTSIVFSLEEGPGQLFKALAVFALRKINLTKMESRPHKKK
PLRIADDNCSAPLKHFDYLFYVDLEASMADPNAQNALANLKEFATFLRVLGSYPTDV
SEA
OrysaADT3
(SEQ ID NO: 77)
RVAYQGVPGAYSEAAAAKAYPSCDAIPCDQFEVAFQAVELWIADRAVLPVENSLGG
SIHRNYDLLLRHRLHIVGEVQLPVHHCLLALPGVRRDLLTRVISHPQALAQCELTLNA
MGLNVAREAFDDTAAAAEHVAAAGLRDTAAIASSRAAELYGLQVLADGIQDDAGN
VTRFVMLAREPIIPRTDRPFKTSIVFAHDREGTSVLFKVLSAFAFRDISLTKIESRPHRH
RPIRLVDDANVGTAKHFEYMFYIDFQASMAEVRAQNALSEIQEFTSFLRVLGSYPMD
MTPWEYSSSGQTAGDTSSS
OrysaADT4
(SEQ ID NO: 78)
RVAYQGVPGAYSEAAAAKAYPSCDAIPCDQFEVAFQAVELWIADRAVLPVENSLGG
SIHRNYDLLLRHRLHIVGEVQLPVHHCLLALPGVRRDLLTRVISHPQALAQCELTLNA
MGLNVAREAFDDTAAAAEHVAAAGLRDTAAIASSRAAELYGLQVLADGIQDDAGN
VTRFVMLAREPIIPRTDRPFKTSIVFAHDREGTSVLFKVLSAFAFRDISLTKIESRPHRH
RPIRLVDDANVGTAKHFEYMFYIDFQASMAEVRAQNALSEIQEFTSFLRVLGSYPMD
MTPWEYSSSSSPSPAPGGDTSR
OrysaADT5
(SEQ ID NO: 79)
RVAYQGVPGAYSEKAAGKAYPGCDAIPCDQFEVAFSAVELWIADRAVLPVENSLGG
SIHRNYDLLLRHRLHIVGEVQLPVHHCLMALPGVRKECLTRVMSHPQALAQCEHTL
TAMGLNVVREAFDDTAGAAEYVAANGLRDTAAIASSRAAELYGMEVLADGIQDDC
GNVTRFVMLAREPIVPRTDRPFKTSIVFAHDKEGTSVLFKVLSAFAFRDITLTKIESRP
HRHRPIRLVDDANVGTAKHFEYMFYVDFQASLAEPRAQNALAEVQEYTSFLRVLGS
YPMDMTPMTAGSSSTVTSDDSSST
OrysaADT6
(SEQ ID NO: 80)
RVAFQGAPGAYSEFAAKTALPGCDTVPCRAFADALAAVDGGAVDRAILPVESTMEG
TALRNYDLLLRHDLVVVQEINLFVHYCLLAMPGVRAAEVRRVISHPMALAHCGRAL
ARLGVDREPVEDTAGAVEMLRSNRMLDTAAIASPRAADLYGLDVLAHGLQDESWN
VTRFLLLSKPPSPVTLPMDADAKTSMVVAHRGGSMMVVLKVLSAFSSRNINLTKLE
VINNNDGGGGGGGAAAGHPVMILDTSARGAPTLRAFPHVLYVDCEGASHDPRVLD
AIKEIERFAVFVRVLGCYAADSNVYDLQ
OrysaADT7
(SEQ ID NO: 81)
HVAYQGSPGTAIEEMVFKAFPDCIAVPCKKFVAAFEAVDSSLADIVVLPIENSSTGSF
HQNYDLLLRHKLHIVQEVQVEIELCLWALPGVQKNDLRTIFSHPEEFAQCEHSLSSLR
VIKKNVDHCAAGAEIISMQNLGDAGVIGNAQAAELYGLNIVECNFQDASPNLTRYLV
LAKTADIPKEYGQYKGRREYDLLHFPIKCPVLQVSCPDLSDKHRFRS
PoptrADTl
(SEQ ID NO: 82)
R VAYQGVPGAY SEAAAGKAYP NCEAIPCDQF EVAFQAVELW IADRAVLPVE
NSLGGSIHRN YDLLLRHRLH IVGEVQLPVH HCLLALPGVR KEYINRVISH
PQALAQCELT LTKLGLQAAR EAVDDTAGAA EYIAANNLRD TAAIASARAA
ELYGMQVLAD GIQDDSSNVT RFVMLAREPI IPRTDRPFKT SIVFAHDKGT
SVLFKVLSAF AFRNISLTKI ESRPHRNRPI RLVDDGNVGT AKHFEYMFYI
DFEASMAEVR AQNALAEVQE FTSFLRVLGS YPMDMTPWCP SRGEDDDDDE KNPF
PoptrADT2
(SEQ ID NO: 83)
R VAYQGVPGAY SEAAAGKAYP NCEAIPCDQF EVAFQAVELW IADRAVLPVE
NSLGGSIHRN YDLLLRHRLH IVGEVQLPVH HCLLALPGVR KEYVNRVISH
PQALAQCELT LTKLGLQAVR EAVDDTAGAA EYIAANNLRD TAAIASARAA
ELYGMQVLAD GIQDDSSNVT RFVMLAREPI IPRTDRPFRT SIVFAHDKGT
SVLFKVLSAF AFRNISLTKI ESRPHRNRPI RLVDDANVGT AKHFEYMFYV
DFEASMAEVR AQNALAEVQE FTSFLRVLGS YPMDMTPWCP SREDDNDDGG
NEKNPF
PoptrADT3
(SEQ ID NO: 84)
RVAYQG IPGAYSEAAA LKAYPKCETV PCDQFEAAFK AVELWLVDKA
VLPIENSVGG SIHRNYDLLLRHRLHIVGEV QMVVNHCLLG LPGVPKEELK
RVLSHPQALA QCEMTLTKLG IIRVSADDSAGAAQMVVANG ERDTGAIASA
RAADIYGLNI LLEKIQDDDD NITRFLILAR EPMIPGSNRPHKTSIVFTLE
EGPGMLFKAL AVFALRDINL TKIESRPQRK RPLRVVDDSN
KGSARYFDYLFYIDFDASMA EPRAQHALAH LQEFARFLRV LGCYPTDATL
PoptrADT4
(SEQ ID NO: 85)
RVAYQGMPG AYGEAAALKA YPKCETVPCE EFEAAFKAVE LWLVDKAVLP
IESSVGGSIHRNYDLLLRHR LHIVGEVQMV VNHCLLGLPG VQKEELKRVL
SHPQALDQCD MILTKLGVVRVSTDDTAGAA LMVAASGERD SGVIASDRAA
EIYGLNILLE KIQDDDDNIT RFLILAREPI IPGTDRPHKT SIVFTLEEGP
GMLFKALAVF ASRDINLTKI ESRPQRKRPL RVVDDSNKGS ARYFDYLFYI
DFEASMAEPR AQHAMAHLQE FASFLRVLGC YATDSAL
PoptrADT5
(SEQ ID NO: 86)
R VAYQGVRGAY SESAAQKAYP NCEAVPCEQF DTAFESVERW LVDRAVLPIE
NSLGGSIHRN YDLLLRHRLH IVGEVKYAVR HCLLANHGVK VEDLKRVLSH
PQALAQCENT LTKLGLVREA VDDTAGAAKH VALQKLEDTG AVASSAAASI
YGLNILAEDI QDDSDNVTRF LILAREPIIP GTDRPFKIQT SIVFSLEEGP
GVLFKALAVF ALRQINLTKI ESRPLRKQPL RASDDGNSGL PKYFDYLFYV
DFEASMADEN AQNALRHLKE FATFLRVLGS YPVHTSMV
P. taeda_ADT1
(SEQ ID NO: 87)
RVAYQGVPGAYSETAALKAYSHCDAVPCEQFEAAFQAVELWLVDKAVLPIENSLGG
SIHRNYDLLLRHRLHIVGEVKLAVHHCLLGLPGVKKEELKRVVSHPQALSQCEHTLS
TLGVIREAVDDTAGAAQFIAANNLRDTGAVASARAAEIYGLQILADGIQDDSDNVTR
FLMLAREPIIPRIDRPFKTSIVFTLEEGPGVLFKALAVFALRDINLTKIESRPQRKRPLRV
VDDSNTGAAKYFDYLFYIDFEASMADPRAQNALGHLQEFATFMRVLGSYPMDMTP
TGRL
P. taeda_ADT2
(SEQ ID NO: 88)
RVAYQGVPGAYSEAAAGKAYPGCEPIPCDQFEAAFQAVELWVADRAVLPVENSLG
GSIHRNYDLLLRHRLHIVGEVQLPVHHCLLTLPGTRKEDVRRVISHPQALAQCEHTIS
KLIGHKVIREGVDDTAGAAQMVAENYLRDTAAIASSRAAEIYGMNIIADGIQDDASN
VTRFVILAREPIIPRVDRPFKTSIVFAQDEGTGILFKVLAAFAFRDISLTKIESRPQRNRP
LRVVDDSNLGNAKYFEYLFYIDFEASMADPRAQNALAELQEFTNFLRVLGSYPMDIS
PPIDSLDSS
P. taeda_ADT3
(SEQ ID NO: 89)
RVAYQGVPGAYSEAAATKAYPSCEPIPCDQFEVAFQAVELWIADRAVLPVENSLGGS
IHRNYDLLLRHSLHIVGEVQFPVHHCLLALPGVKPENLRRVISHPQALAQCENTLTLM
GLNVAREAFDDTAGAAEFVAANDLRDTASIASARAAEIYGMNILADGIQDDVGNVT
RFVMLAREPVIPRTDRPFKTSIVFAHEEGTGVLFKVLSAFAFRNINLTKIESRPQRSKP
VRVVDDVNGGTAKHFFEYIFYVDFEASMANPRAQNALAEVQEFTTFLRVLGSYPMD
IAPLNRGNSCSNHH
P. taeda_ADT4
(SEQ ID NO: 90)
RVAYQGVRGSYCQEAAVRAFQRCDALPCEGEMESAFEALESDAADRAVVPVENSL
DGVIGRNYDLILRHPDLHVVGEILLPINHCLLAVRGAGKRRTLKRVISHPQALAHCQR
CLGDLGVEVEGVNNAASAARFVAENRIDDTAVIGSAIAGQEYGLEVVEEEMQDDSF
NTTRFLIFTKKKPNNNSSAESGLKTTVAFSLKEGTADLCKALSIFAARDIKVTKIESRP
LRENPLRMVTNEEQEGSSSKCYFKYVFFVDLEAPVADDHPGQVQMALDSLRHIARF
VRVIGNYSVLSVL
E. coli_P-Protein
(SEQ ID NO: 91)
RIAFLGPKGSYSHLAARQYAARHFEQFIESGCAKFADIFNQVETGQADYAVVPIENTS
SGAINDVYDLLQHTSLSIVGEMTLTIDHCLLVSGTTDLSAINTVYSHPQPFQQCSKFLN
RYPHWKIEYTESTSAAMEKVAQAKSPHVAALGSEAGGTLYGLQVLERIEANQRQNF
TRFVVLARKAINVSDQVPAKTTLLMATGQQAGALVEALLVLRNHSLIMTRLESRPIH
GNPWEEMFYLDIQANLESAEMQKALKELGEITRSMKVLGCYPENVVPVDPT
VitviADT1
(SEQ ID NO: 107)
RVAYQGVPGAYSEAAAGKAYPNCEAIPCDQFEVAFQAVELWIADRAVLPVENSLGG
SIHRNYDLLLRHRLHIVGEVQLPVHHCLLALPGVRKEYLTRVISHPQALAQCEHTLTK
LGLNVAREAVDDTAGAAEYVALNNLRDTAAIASARAADLYGLQILADGIQDDSSNV
TRFVMLAREPIIPRTDRPFKTSIVFAHDKGTSVLFKVLSAFAFRNISLTKIESRPHRNRPI
RLVDDANVGTAKHFEYMFYVDFEASMAEVRAQNALAEVQEFTSFLRVLGSYPMDM
TPWCPSGGD
VitviADT2
(SEQ ID NO: 108)
RVAYQGVHGAYSESAAEKAYPNCQAVPCEQFETAFEAVESWLVDRAVLPIENSLGG
SIHRNYDLLLRHRLHIVGEVKFAVRHCLLANHGVKVEDLKRVLSHSQALAQCENTL
TKLGLVREAVDDTAGAAKFIAFHKLKDVGAVASSAAARIYGLKILAQDIQDDSYNV
TRFLMLAREPIIPGTDRPFKTSIVFSLEEGPGVLFKALAVFALRQINLTKIESRPLRNQP
LRASNDTNNGSPKYFDYLFYVDFEASMADQNSQNALRHLKEFATFLRVLGSYPVDT
SMI
VitviADT3
(SEQ ID NO: 109)
RVAYQGAPGAYSEEAAMKAYPKCEAVPCDDFEAAFKAVELWLVEKAVLPIENSVG
GSIHRNYDLLLGHRLHIVGEVQMVVNHCLLGLPGVRKDELKRVLSHPQAFAQCDMT
LNELGLLRISTEDTAGAAQIVASDGLKNTGAIASARAAVIYGLNILEEKIQDDCDNITR
FLILAREPIIPGLERPYKTSIVFSLDEGPGVLFKALAVFALRDISLSKIESRPQRKRPLRIV
DDSNKGSAKYFDYLFYIDFEASMAEPRAQYALGHLQEFARFLRVLGCYPMDQTPYE
ATSVDNAGPAAGNS
VitviADT4
(SEQ ID NO: 110)
RISYKGVPGAYSEDAALKAYPHCETVPCDEFEDAFKAVELWLAEKAVLPIENSLGGS
IHRNYDLLLRHRLHIVGEVQLAVNLCLLAIPGVGIDQLRRVLSHPQALAQSDIILSKLG
VSRENVDDSAGAAQYVASHGLRDAGVVASARAAEIYGLNILAERIQDDFDNITRFLV
LARDPIIPRTNKLFKTSIVFTLEEGPGVLFKALAVFALRDINLTKIESRPQRKKPLRVVD
DSNTGSAKYFDYLFYIDFEASMAEPRAQTALAHLQEFATFLRVLGCYPMDSFP

TABLE 8
% identity comparison

| Seq | Arabidopsis | | | | | | Rice | | | | | Sitka | | |
|---|---|---|---|---|---|---|---|---|---|---|---|---|---|---|
| Names | ADT1 | ADT2 | ADT3 | ADT4 | ADT5 | ADT6 | ADT1 | ADT2 | ADT3 | ADT4 | ADT5 | ADT1 | ADT2 | ADT3 |
| ArathADT1 | ID | 0.6 | 0.5 | 0.5 | 0.5 | 0.6 | 0.6 | 0.6 | 0.5 | 0.5 | 0.5 | 0.7 | 0.6 | 0.6 |
| | | 62 | 94 | 85 | 85 | 19 | 44 | 38 | 9 | 84 | 68 | 51 | 39 | |
| ArathADT2 | 0.6 | ID | 0.6 | 0.6 | 0.6 | 0.6 | 0.7 | 0.7 | 0.6 | 0.6 | 0.6 | 0.7 | 0.6 | 0.6 |
| | 62 | | 36 | 18 | 25 | 59 | 1 | 32 | 06 | | 04 | 48 | 53 | 42 |
| ArathADT3 | 0.5 | 0.6 | ID | 0.7 | 0.7 | 0.9 | 0.6 | 0.6 | 0.8 | 0.8 | 0.8 | 0.7 | 0.7 | 0.8 |
| | 94 | 36 | | 71 | 84 | 3 | 19 | 27 | 39 | 31 | 26 | 19 | 59 | 55 |
| ArathADT4 | 0.5 | 0.6 | 0.7 | ID | 0.8 | 0.8 | 0.6 | 0.6 | 0.7 | 0.7 | 0.7 | 0.7 | 0.7 | 0.7 |
| | 85 | 18 | 71 | | 99 | 2 | 11 | 22 | 81 | 76 | 69 | 05 | 23 | 75 |
| ArathADT5 | 0.5 | 0.6 | 0.7 | 0.8 | ID | 0.8 | 0.5 | 0.6 | 0.7 | 0.7 | 0.7 | 0.7 | 0.7 | 0.7 |
| | 85 | 25 | 84 | 99 | | 2 | 98 | 18 | 85 | 79 | 59 | 02 | 16 | 85 |
| ArathADT6 | 0.6 | 0.6 | 0.9 | 0.8 | 0.8 | ID | 0.6 | 0.6 | 0.8 | 0.8 | 0.8 | 0.7 | 0.7 | 0.8 |
| | 19 | 59 | 3 | 2 | 2 | | 45 | 56 | 36 | 31 | 39 | 5 | 85 | 59 |
| OrysaADT1 | 0.6 | 0.7 | 0.6 | 0.6 | 0.5 | 0.6 | ID | 0.8 | 0.6 | 0.6 | 0.6 | 0.7 | 0.6 | 0.6 |
| | 44 | 1 | 19 | 11 | 98 | 45 | | 65 | 18 | 12 | 16 | 16 | 35 | 35 |
| OrysaADT2 | 0.6 | 0.7 | 0.6 | 0.6 | 0.6 | 0.6 | 0.8 | ID | 0.6 | 0.6 | 0.6 | 0.7 | 0.6 | 0.6 |
| | 38 | 32 | 27 | 22 | 18 | 56 | 65 | | 22 | 16 | 14 | 18 | 56 | 45 |
| OrysaADT3 | 0.5 | 0.6 | 0.8 | 0.7 | 0.7 | 0.8 | 0.6 | 0.6 | ID | 0.9 | 0.8 | 0.7 | 0.7 | 0.8 |
| | 9 | 06 | 39 | 81 | 85 | 36 | 18 | 22 | | 67 | 75 | 01 | 35 | 36 |
| OrysaADT4 | 0.5 | 0.6 | 0.8 | 0.7 | 0.7 | 0.8 | 0.6 | 0.6 | 0.9 | ID | 0.8 | 0.6 | 0.7 | 0.8 |
| | 84 | | 31 | 76 | 79 | 31 | 12 | 16 | 67 | | 64 | 94 | 34 | 29 |
| OrysaADT5 | 0.5 | 0.6 | 0.8 | 0.7 | 0.7 | 0.8 | 0.6 | 0.6 | 0.8 | 0.8 | ID | 0.6 | 0.7 | 0.8 |
| | 68 | 04 | 26 | 69 | 59 | 39 | 16 | 14 | 75 | 64 | | 89 | 45 | 37 |
| PicsiADT1 | 0.7 | 0.7 | 0.7 | 0.7 | 0.7 | 0.7 | 0.7 | 0.7 | 0.7 | 0.6 | 0.6 | ID | 0.7 | 0.7 |
| | 51 | 48 | 19 | 05 | 02 | 5 | 16 | 18 | 01 | 94 | 89 | | 54 | 31 |
| PicsiADT2 | 0.6 | 0.6 | 0.7 | 0.7 | 0.7 | 0.7 | 0.6 | 0.6 | 0.7 | 0.7 | 0.7 | 0.7 | ID | 0.7 |
| | 39 | 53 | 59 | 23 | 16 | 85 | 35 | 56 | 35 | 34 | 45 | 54 | | 64 |
| PicsiADT3 | 0.6 | 0.6 | 0.8 | 0.7 | 0.7 | 0.8 | 06 | 0.6 | 0.8 | 0.8 | 0.8 | 0.7 | 0.7 | ID |
| | | 42 | 55 | 75 | 85 | 59 | 35 | 45 | 36 | 29 | 37 | 31 | 64 | |
| PoptrADT1 | 0.6 | 0.6 | 0.8 | 0.7 | 0.8 | 0.8 | 0.6 | 0.6 | 0.8 | 0.8 | 0.8 | 0.7 | 0.7 | 0.8 |
| | | 32 | 63 | 98 | 14 | 72 | 28 | 36 | 44 | 38 | 28 | 27 | 61 | 7 |
| PoptrADT2 | 0.5 | 0.6 | 0.8 | 0.7 | 0.8 | 0.8 | 0.6 | 0.6 | 0.8 | 0.8 | 0.8 | 0.7 | 0.7 | 0.8 |
| | 86 | 28 | 51 | 87 | | 66 | 21 | 31 | 32 | 32 | 32 | 16 | 5 | 58 |
| PoptrADT3 | 0.7 | 0.6 | 0.6 | 0.6 | 0.6 | 0.6 | 0.6 | 0.6 | 0.6 | 0.6 | 0.6 | 0.8 | 0.7 | 0.6 |
| | 65 | 93 | 43 | 18 | 12 | 7 | 68 | 66 | 29 | 23 | 27 | 06 | | 32 |
| PoptrADT4 | 0.7 | 0.6 | 0.6 | 0.5 | 0.5 | 0.6 | 0.6 | 0.6 | 0.6 | 0.6 | 0.6 | 0.7 | 0.6 | 0.6 |
| | 51 | 82 | 27 | 98 | 95 | 53 | 58 | 52 | 16 | 1 | 11 | 79 | 8 | 22 |
| PoptrADT5 | 0.6 | 0.8 | 0.6 | 0.6 | 0.6 | 0.6 | 0.7 | 0.7 | 0.6 | 0.6 | 0.6 | 0.7 | 0.6 | 0.6 |
| | 92 | 61 | 32 | 21 | 24 | 58 | 39 | 58 | 22 | 16 | 13 | 63 | 72 | 54 |
| VitviADT1 | 0.6 | 0.6 | 0.8 | 0.8 | 0.8 | 0.9 | 0.6 | 0.6 | 0.8 | 0.8 | 0.8 | 0.7 | 0.7 | 0.8 |
| | 23 | 67 | 84 | 16 | 29 | 19 | 56 | 67 | 59 | 5 | 49 | 52 | 91 | 68 |
| VitviADT2 | 0.6 | 0.8 | 0.6 | 0.6 | 0.6 | 0.6 | 0.7 | 0.7 | 0.6 | 0.6 | 0.6 | 0.7 | 0.6 | 0.6 |
| | 93 | 53 | 5 | 25 | 28 | 76 | 2 | 5 | 19 | 13 | 14 | 65 | 66 | 59 |
| VitviADT3 | 0.7 | 0.6 | 0.6 | 0.6 | 0.6 | 0.6 | 0.6 | 0.6 | 0.6 | 0.6 | 0.6 | 0.7 | 0.6 | 0.6 |
| | 11 | 52 | 14 | 14 | 01 | 49 | 19 | 07 | 29 | 29 | 11 | 45 | 62 | 13 |
| VitviADT4 | 0.7 | 0.7 | 0.6 | 0.6 | 0.6 | 0.6 | 0.6 | 0.6 | 0.6 | 0.6 | 0.6 | 0.7 | 0.6 | 0.6 |
| | 16 | 17 | 23 | 25 | 32 | 59 | 68 | 87 | 26 | 2 | 14 | 82 | 8 | 22 |

| Seq | Poplar | | | | | Grape | | | |
|---|---|---|---|---|---|---|---|---|---|
| Names | ADT1 | ADT2 | ADT3 | ADT4 | ADT5 | ADT1 | ADT2 | ADT3 | ADT4 |
| ArathADT1 | 0.6 | 0.5 | 0.7 | 0.7 | 0.6 | 0.6 | 0.6 | 0.7 | 0.7 |
| | | 86 | 65 | 51 | 92 | 23 | 93 | 11 | 16 |
| ArathADT2 | 0.6 | 0.6 | 0.6 | 0.6 | 0.8 | 0.6 | 0.8 | 0.6 | 0.7 |
| | 32 | 28 | 93 | 82 | 61 | 67 | 53 | 52 | 17 |
| ArathADT3 | 0.8 | 0.8 | 0.6 | 0.6 | 0.6 | 0.8 | 0.6 | 0.6 | 0.6 |
| | 63 | 51 | 43 | 27 | 32 | 84 | 5 | 14 | 23 |
| ArathADT4 | 0.7 | 0.7 | 0.6 | 0.5 | 0.6 | 0.8 | 0.6 | 0.6 | 0.6 |
| | 98 | 87 | 18 | 98 | 21 | 16 | 25 | 14 | 25 |
| ArathADT5 | 0.8 | 0.8 | 0.6 | 0.5 | 0.6 | 0.8 | 0.6 | 0.6 | 0.6 |
| | 14 | | 12 | 95 | 24 | 29 | 28 | 01 | 32 |
| ArathADT6 | 0.8 | 0.8 | 0.6 | 0.6 | 0.6 | 0.9 | 0.6 | 0.6 | 0.6 |
| | 72 | 66 | 7 | 53 | 58 | 19 | 76 | 49 | 59 |
| OrysaADT1 | 0.6 | 0.6 | 0.6 | 0.6 | 0.7 | 0.6 | 0.7 | 0.6 | 0.6 |
| | 28 | 21 | 68 | 58 | 39 | 56 | 2 | 19 | 68 |
| OrysaADT2 | 0.6 | 0.6 | 0.6 | 0.6 | 0.7 | 0.6 | 0.7 | 0.6 | 0.6 |
| | 36 | 31 | 66 | 52 | 58 | 67 | 5 | 07 | 87 |
| OrysaADT3 | 0.8 | 0.8 | 0.6 | 0.6 | 0.6 | 0.8 | 0.6 | 0.6 | 0.6 |
| | 44 | 32 | 29 | 16 | 22 | 59 | 19 | 29 | 26 |
| OrysaADT4 | 0.8 | 0.8 | 0.6 | 0.6 | 0.6 | 0.8 | 0.6 | 0.6 | 0.6 |
| | 38 | 32 | 23 | 1 | 16 | 5 | 13 | 29 | 2 |
| OrysaADT5 | 0.8 | 0.8 | 0.6 | 0.6 | 0.6 | 0.8 | 0.6 | 0.6 | 0.6 |
| | 28 | 32 | 27 | 11 | 13 | 49 | 14 | 11 | 14 |
| PicsiADT1 | 0.7 | 0.7 | 0.8 | 0.7 | 0.7 | 0.7 | 0.7 | 0.7 | 0.7 |
| | 27 | 16 | 06 | 79 | 63 | 52 | 65 | 45 | 82 |
| PicsiADT2 | 0.7 | 0.7 | 0.7 | 0.6 | 0.6 | 0.7 | 0.6 | 0.6 | 0.6 |
| | 61 | 5 | | 8 | 72 | 91 | 66 | 62 | 8 |
| PicsiADT3 | 0.8 | 0.8 | 0.6 | 0.6 | 0.6 | 0.8 | 0.6 | 0.6 | 0.6 |
| | 7 | 58 | 32 | 22 | 54 | 68 | 59 | 13 | 22 |
| PoptrADT1 | ID | 0.9 | 0.6 | 0.6 | 0.6 | 0.9 | 0.6 | 0.6 | 0.6 |
| | | 64 | 45 | 26 | 38 | 21 | 42 | 25 | 36 |
| PoptrADT2 | 0.9 | ID | 0.6 | 0.6 | 0.6 | 0.9 | 0.6 | 0.6 | 0.6 |
| | 64 | | 35 | 15 | 31 | 15 | 38 | 2 | 25 |
| PoptrADT3 | 0.6 | 0.6 | ID | 0.8 | 0.7 | 0.6 | 0.7 | 0.7 | 0.7 |
| | 45 | 35 | | 95 | 19 | 77 | | 94 | 83 |
| PoptrADT4 | 0.6 | 0.6 | 0.8 | ID | 0.7 | 0.6 | 0.6 | 0.7 | 0.7 |
| | 26 | 15 | 95 | | 09 | 54 | 93 | 68 | 83 |
| PoptrADT5 | 0.6 | 0.6 | 0.7 | 0.7 | ID | 0.6 | 0.8 | 0.6 | 0.7 |
| | 38 | 31 | 19 | 09 | | 8 | 96 | 74 | 09 |
| VitviADT1 | 0.9 | 0.9 | 0.6 | 0.6 | 0.6 | ID | 0.6 | 0.6 | 0.6 |
| | 21 | 15 | 77 | 54 | 8 | | 77 | 51 | 64 |
| VitviADT2 | 0.6 | 0.6 | 0.7 | 0.6 | 0.8 | 0.6 | ID | 0.6 | 0.7 |
| | 42 | 38 | | 93 | 96 | 77 | | 58 | |
| VitviADT3 | 0.6 | 0.6 | 0.7 | 0.7 | 0.6 | 0.6 | 0.6 | ID | 0.7 |
| | 25 | 2 | 94 | 68 | 74 | 51 | 58 | | 28 |
| VitviADT4 | 0.6 | 0.6 | 0.7 | 0.7 | 0.7 | 0.6 | 0.7 | 0.7 | ID |
| | 36 | 25 | 83 | 83 | 09 | 64 | | 28 | |

Literature cited and incorporated herein by reference for their respective cited teachings:
1. Cho, M.-H., Corea, O. R. A., Yang, H., Bedgar, D. L., Laskar, D. D., Anterola, A. M., Moog-Anterola, F. A., Hood, R. L., Kohalmi, S. E., Bernards, M. A., Kang, C., Davin, L. B., and Lewis, N. G. (2007) J. Biol. Chem. 282, 30827-30835
2. Jung, E., Zamir, L. O., and Jensen, R. A. (1986) Proc. Natl. Acad. Sci. USA 83, 7231-7235
3. Siehl, D. L., and Conn, E. E. (1988) Arch. Biochem. Biophys. 260, 822-829
4. Lewis, N. G., and Yamamoto, E. (1989) Tannins: Their place in plant metabolism. in Chemistry and Significance of Condensed Tannins (Hemingway, R. W., and Karchesy, J. J. eds.), Plenum Press. pp 23-46
5. van Heerden, P. S., Towers, G. H. N., and Lewis, N. G. (1996) J. Biol. Chem. 271, 12350-12355
6. Vassao, D. G., Kim, K.-W., Davin, L. B., and Lewis, N. G. (2010) Lignans (neolignans) and allyl/propenyl phenols: Biogenesis, structural biology, and biological/human health considerations. in Comprehensive Natural Products II Chemistry and Biology (Mander, L., and Lui, H.-W. eds.), Elsevier, Oxford, UK. pp 815-928
7. Davin, L. B., Jourdes, M., Patten, A. M., Kim, K.-W., Vassao, D. G., and Lewis, N. G. (2008) Nat. Prod. Rep. 25, 1015-1090
8. Bernards, M. A., and Lewis, N. G. (1998) Phytochemistry 47, 915-933
9. Franke, R., and Schreiber, L. (2007) Curr Opin Plant Biol 10, 252-259
10. Anterola, A. M., and Lewis, N. G. (2002) Phytochemistry 61, 221-294
11. Anterola, A. M., van Rensburg, H., van Heerden, P. S., Davin, L. B., and Lewis, N. G. (1999) Biochem. Biophys. Res. Commun. 261, 652-657
12. Anterola, A. M., Jeon, J.-H., Davin, L. B., and Lewis, N. G. (2002) J. Biol. Chem. 277, 18272-18280
13. Rippert, P., Puyaubert, J., Grisollet, D., Derrier, L., and Matringe, M. (2009) Plant Physiol. 149, 1251-1260
14. Yamada, T., Matsuda, F., Kasai, K., Fukuoka, S., Kitamura, K., Tozawa, Y., Miyagawa, H., and Wakasa, K. (2008) Plant Cell 20, 1316-1329
15. Maeda, H., Shasany, A. K., Schnepp, J., Orlova, I., Taguchi, G., Cooper, B. R., Rhodes, D., Pichersky, E., and Dudareva, N. (2010) Plant Cell 22, 832-849
16. Huang, T., Tohge, T., Lytovchenko, A., Fernie, A. R., and Jander, G. Plant J 63, 823-835
17. Patten, A. M., Jourdes, M., Cardenas, C. L., Laskar, D. D., Nakazawa, Y., Chung, B.-Y., Franceschi, V. R., Davin, L. B., and Lewis, N. G. (2010) Mol. BioSyst. 6, 499-515
18. Jourdes, M., Cardenas, C. L., Laskar, D. D., Moinuddin, S. G. A., Davin, L. B., and Lewis, N. G. (2007) Phytochemistry 68, 1932-1956
19. Corea, O. R. A., Ki, C., Cardenas, C. L., Patten, A. M., Davin, L. B., and Lewis, N. G. (2010) Joint Annual Meeting of the American Society of Pharmacognosy and the Phytochemical Society of North America, St. Petersburg Beach, Fla.; July 2010
20. Corea, O. R. A., Ki, C., Patten, A. M., Cardenas, C. L., Davin, L. B., and Lewis, N. G. (2010) Plant Biology 2010, Montreal, Quebec, Canada; July 2010
21. Ralph, J., Lundquist, K., Brunow, G., Lu, F., Kim, H., Schatz, P. F., Marita, J. M., Hatfield, R. D., Ralph, S. A., Christensen, J. H., and Boerjan, W. (2004) Phytochem. Rev. 3, 29-60
22. Tan, K., Li, H., Zhang, R., Gu, M., Clancy, S. T., and Joachimiak, A. (2008) J. Struct. Biol. 162, 94-107
23. Laskar, D. D., Corea, O. R. A., Patten, A. M., Kang, C., Davin, L. B., and Lewis, N. G. (2010) Vascular plant lignification: Biochemical/structural biology considerations of upstream aromatic amino acid and monolignol pathways. in Comprehensive Natural Products II Chemistry and Biology (Mander, L., and Lui, H.-W. eds.), Elsevier, Oxford, UK. pp 541-604
24. Ehlting, J., Mattheus, N., Aeschliman, D. S., Li, E., Hamberger, B., Cullis, I. F., Zhuang, J., Kaneda, M., Mansfield, S. D., Samuels, L., Ritland, K., Ellis, B. E., Bohlmann, J., and Douglas, C. J. (2005) Plant J. 42, 618-640
25. Ehlting, J., Sauveplane, V., Olry, A., Ginglinger, J.-F., Provart, N. J., and Werck-Reichhart, D. (2008) BMC Plant Biol 8, 47 (http://www.biomedcentral.com/1471-2229/1478/1447)
26. Mutwil, M., Øbro, J., Willats, W. G., and Persson, S. (2008) Nucleic Acids Res. 36, W320-W326
27. Toufighi, K., Brady, S. M., Austin, R., Ly, E., and Provart, N. J. (2005) Plant J. 43, 153-163
28. Yokoyama, A., Yamashino, T., Amano, Y., Tajima, Y., Imamura, A., Sakakibara, H., and Mizuno, T. (2007) Plant Cell Physiol 48, 84-96
29. Lim, E. K., Li, Y., Parr, A., Jackson, R., Ashford, D. A., and Bowles, D. J. (2001) J Biol Chem 276, 4344-4349
30. Solfanelli, C., Poggi, A., Loreti, E., Alpi, A., and Perata, P. (2006) Plant Physiol 140, 637-646
31. Peng, M., Hudson, D., Schofield, A., Tsao, R., Yang, R., Gu, H., Bi, Y. M., and Rothstein, S. J. (2008) J Exp Bot 59, 2933-2944
32. Kubo, H., Nawa, N., and Lupsea, S. A. (2007) J Plant Res 120, 445-449
33. Stepansky, A., and Leustek, T. (2006) Amino Acids 30, 127-142
34. Alonso, J. M., Stepanova, A. N., Leisse, T. J., Kim, C. J., Chen, H., Shinn, P., Stevenson, D. K., Zimmerman, J., Barajas, P., Cheuk, R., Gadrinab, C., Heller, C., Jeske, A., Koesema, E., Meyers, C. C., Parker, H., Prednis, L., Ansari, Y., Choy, N., Deen, H., Geralt, M., Hazari, N., Hom, E., Karnes, M., Mulholland, C., Ndubaku, R., Schmidt, I., Guzman, P., Aguilar-Henonin, L., Schmid, M., Weigel, D., Carter, D. E., Marchand, T., Risseeuw, E., Brogden, D., Zeko, A., Crosby, W. L., Berry, C. C., and Ecker, J. R. (2003) Science 301, 653-657
35. Samson, F., Brunaud, V., Balzergue, S., Dubreucq, B., Lepiniec, L., Pelletier, G., Caboche, M., and Lecharny, A. (2002) Nucleic Acids Res. 30, 94-97
36. Czechowski, T., Stitt, M., Altmann, T., Udvardi, M. K., and Scheible, W. R. (2005) Plant Physiol 139, 5-17
37. Corea, O. R. A., Ki, C., Cardenas, C. L., Davin, L. B., and Lewis, N. G. (2011) Methods in Molecular Biology, (Manuscript submitted)
38. Ruzin, S. E. (1999) Plant Microtechnique and Microscopy, Oxford University Press, New York.
39. Wiesner, J. (1878) Sitzungsberichte der Kaiserlichen Akademie der Wissenschaften. Mathematisch-Naturwissenschaftliche Classe 77, 60-66
40. Mäule, C. (1901) Beiträge zur wissenschaftlichen Botanik 4, 166-185
41. Laskar, D. D., Jourdes, M., Patten, A. M., Helms, G. L., Davin, L. B., and Lewis, N. G. (2006) Plant J. 48, 674-686
42. Patten, A. M., Cardenas, C. L., Cochrane, F. C., Laskar, D. D., Bedgar, D. L., Davin, L. B., and Lewis, N. G. (2005) Phytochemistry 66, 2092-2107
43. Iiyama, K., and Wallis, A. F. A. (1990) J. Sci. Food Agr. 51, 145-161
44. Blee, K., Choi, J. W., O'Connell, A. P., Jupe, S. C., Schuch, W., Lewis, N. G., and Bolwell, G. P. (2001) Phytochemistry 57, 1159-1166
45. Lapierre, C., Monties, B., and Rolando, C. (1986) Holzforschung 40, 113-118
46. Rolando, C., Monties, B., and Lapierre, C. (1992) Thioacidolysis. in Methods in Lignin Chemistry (Lin, S. Y., and Dence, C. W. eds.), Springer, Berlin. pp 334-349
47. Faix, 0., Meier, D., and Fortmann, I. (1990) Holz Roh-Werkst. 48, 351-354
48. Faix, 0., Meier, D., and Fortmann, I. (1990) Holz Roh-Werkst. 48, 281-285
49. Altamura, M. M., Possenti, M., Matteucci, A., Baima, S., Ruberti, I., and Morelli, G. (2001) New Phytol. 151, 381-389
50. Miesak, B. H., and Coruzzi, G. M. (2002) Plant Physiol 129, 650-660
51. Karimi, M., Inze, D., and Depicker, A. (2002) Trends in Plant Science 7, 193-195
52. An, G., Ebert, P. R., Mitra, A., and Ha, S. B. (1988) Binary Vectors. in Plant Molecular Biology Manual (Gelvin, S. B., Schilperoort, R. A., and Verma, D. P. S. eds.), Kluwer Academic Publishers, Dordrecht. pp 1-19
53. Filichkin, S. A., Meilan, R., Busov, V. B., Ma, C., Brunner, A. M., and Strauss, S. H. (2006) Plant Cell Rep 25, 660-667
54. Tsai, C. J., Harding, S. A., Tschaplinski, T. J., Lindroth, R. L., and Yuan, Y. (2006) New Phytol 172, 47-62

1. A method for decreasing carbon flow into lignin in plants, comprising:
obtaining a plant or cell thereof having at least one chloroplast-localized arogenate dehydratase (ADT) and having cytosolic and/or membrane-associated phenylpropanoid metabolism for producing phenyalanine (Phe)-derived phenylpropanoids; and
reducing or eliminating, using at least one of mutagenesis and recombinant means, in the plant or the cell thereof, expression and/or activity of the at least one chloroplast-localized ADT sufficient to reduce Phe availability for metabolism into Phe-derived phenylpropanoids, wherein the amount, level or distribution of lignin in the plant or the cell thereof is reduced relative to control plants or cells thereof with normal amounts, levels or distributions of lignin, and wherein a method for decreasing carbon flow into lignin in a plant or cell thereof is afforded.

2. The method of claim 1, wherein the plant has a plurality of chloroplast-localized arogenate dehydratases (ADTs), and wherein reducing or eliminating comprises reducing or eliminating expression and/or activity of at least two of the plurality of chloroplast-localized ADTs sufficient to reduce Phe availability for metabolism into Phe-derived phenylpropanoids, wherein the amount, level or distribution of lignin in the plant or the cell thereof is reduced relative to control plants or cells thereof with normal amounts, levels or distributions of lignin, preferably wherein reducing or eliminating comprises reducing or eliminating expression and/or activity of at least three of the plurality of chloroplast-localized ADTs, preferably wherein reducing or eliminating comprises reducing or eliminating expression and/or activity of at least four of the plurality of chloroplast-localized ADTs, preferably wherein reducing or eliminating is by knock-out of chloroplast-localized ADTs.

3. The method of claim 2, wherein the at least two of the plurality of chloroplast-localized arogenate dehydratases (ADTs) correspond to phylogenetic subgroup III chloroplast-localized ADTs as defined herein, preferably wherein at least three of the plurality of chloroplast-localized arogenate dehydratases (ADTs) correspond to phylogenetic subgroup III chloroplast-localized ADTs as defined herein.

4. The method of claim 1, wherein the at least one chloroplast-localized arogenate dehydratase (ADT) comprises a conserved TRF motif in the ADT active site.

5. The method of claim 1, wherein at least one chloroplast-localized arogenate dehydratase (ADT) comprises at least one sequence selected from the group consisting of SEQ ID NOS:30, 32, 34, 36, 38, 40, 42, 44, 46, 48, 93, 50, 52, 56, 58, 60, 62, 64, 66, 68, 98, 100, 102, 104, 106 orthologs thereof, a sequence having at least 45%, at least 50%, at least 55%, at least 60%, at least 65%, at least 70%, at least 80%, at least 85%, or at least 95% amino acid sequence identity therewith, and ADT-active portions thereof, preferably with at least 60% amino acid sequence identity therewith.

6. The method of claim 1, wherein the chloroplast-localized arogenate dehydratase (ADT) is that of a vascular plant.

7. The method of claim 1, wherein the plant or cell thereof is selected from the group consisting of hardwood, softwood, graminae and angiosperms.

8. The method of claim 1, wherein the plant or cell thereof is selected from the group consisting of Arabidopsis, poplar, Populus trichocarpa, pine, Pinus taeda, rice, Oryza sativa, Picea sitchensis, and Vitis vinifera.

9. The method of any one of claims 1 through 8, wherein reducing or eliminating comprises use of at least one of gene-silencing, gene knock-out, anti-sense methods, siRNA methods, RNAi methods, and transgenic methods.

10. The method of claim 1, wherein reducing or eliminating comprises inactivating a gene normally encoding the at least one chloroplast-localized arogenate dehydratase (ADT).

11. The method of claim 1, wherein reducing or eliminating comprises expression of RNAi sufficient to expression and/or activity of the at least one chloroplast-localized arogenate dehydratase (ADT).

12. The method of claim 1, comprising, imparting into the germplasm of a plant variety a mutation or genetic alteration that reduces the expression or activity of the at least one chloroplast-localized arogenate dehydratase (ADT) in one or more cells of the plant, wherein the amount or level of lignin is reduced relative to control plants or cells thereof with normal amounts or levels of lignin, preferably comprising introducing into the selected variety using suitable methods a transgene that reduces the expression or activity of the at least one chloroplast-localized ADT in one or more cells of the plant relative to that of control plants or cells thereof.

13. The method of claim 12, comprising the use of T-DNA insertion.

14. A recombinant plant or a part or cell thereof, comprising at least one mutation, genetic alteration or transgene that reduces or eliminates the expression and/or activity of at least one chloroplast-localized arogenate dehydratase (ADT) in one or more cells of the plant, wherein the amount, level or distribution of lignin in the plant or the cell thereof is reduced relative to control plants or cells thereof with normal amounts, levels or distributions of lignin.

15. The plant or part or cell thereof of claim 14, wherein the plant has a plurality of chloroplast-localized arogenate dehydratases (ADTs), and wherein the expression and/or activity of at least two of the chloroplast-localized ADTs is reduced or eliminated, preferably wherein the expression and/or activity of at least three of the chloroplast-localized ADTs is reduced or eliminated, preferably wherein the expression and/or activity of at least four of the chloroplast-localized (ADTs is reduced or eliminated.

16. The plant or part or cell thereof of claim 14, wherein the plant or part thereof is at least one of Arabidopsis, poplar, Populus trichocarpa, pine, Pinus taeda, rice, Oryza sativa, Picea sitchensis, and Vitis vinifera.

17. The plant or part or cell thereof of claim 16, wherein the plant or part thereof is other than Arabidopsis.

18. The plant or part or cell thereof of any one of claims 14 through 17, wherein the at least one mutation, genetic alteration or transgene that reduces or eliminates the expression and/or activity of at least one chloroplast-localized arogenate dehydratase (ADT) comprises at least one of gene-silencing, gene knock-out, anti-sense methods, siRNA methods, RNAi methods, and transgenic methods.

19. A seed or true-breeding seed derived from the recombinant plant or a part thereof of any one of claims 14 through 17.

20. A reduced lignin plant product derived from the plant of any one of claims 14 through 17, preferably wherein the plant product has less lignin relative to that of plant product derived from control plants or cells thereof with normal amounts, levels or distributions of lignin.

21. The reduced lignin plant product of claim 20, comprising at least one of a fuel, food composition, antioxidant, and feedstock.
